(19) United States
(12) Patent Application Publication
(10) Pub. No.: US 2023/0099822 A1
(43) Pub. Date: Mar. 30, 2023

(54) MR1 LIGANDS AND PHARMACEUTICAL COMPOSITIONS FOR IMMUNOMODULATION

(71) Applicant: UNIVERSITÄT BASEL, Basel (CH)

(72) Inventors: Gennaro DE LIBERO, Basel (CH); Lucia MORI, Basel (CH); Alessandro VACCHINI, Basel (CH); Andrew CHANCELLOR, Basel (CH); Julian SPAGNUOLO, Basel (CH); Qinmei YANG, Basel (CH)

(73) Assignee: UNIVERSITÄT BASEL, Basel (CH)

(21) Appl. No.: 17/793,419

(22) PCT Filed: Jan. 18, 2021
(86) PCT No.: PCT/EP2021/050972

§ 371 Date: Jul. 18, 2022

(30) Foreign Application Priority Data
Jan. 16, 2020 (EP) 20152326.3
Mar. 30, 2020 (EP) 20166918.1
Mar. 30, 2020 (EP) 20166919.9

(51) Int. Cl. G01N 33/50; G01N 33/574; C07D 239/70

(52) U.S. Cl. CPC G01N 33/505; G01N 33/574; C07D 239/70; G01N 2800/7028

(57) ABSTRACT

The invention relates to a method for modulating an interaction between an MR1 polypeptide and an MR1-specific T cell receptor molecule, whereby a MR1 polypeptide is contacted with a MR1 ligand compound that is a nucleobase adduct product reflecting a state of metabolic distress of a cell.

The invention further relates to the use of compounds identified as MR1 ligands in vaccination or modulation of an MR1-restricted immune response.

This application claims the benefit of European Patent Applications EP20152326.3, filed 16 Jan. 2020, EP20166918.1 filed 30 Mar. 2020 and EP20166919.9, filed 30 Mar. 2020, all of which are incorporated herein by reference.

The present invention relates to ligands specifically presented by MR1 molecules to MR1-specific T cells. These ligands are derivatives or analogues of nucleic acid forming bases, particularly ribonucleoside and deoxyribonucleoside adducts occurring in eukaryotic cells under certain conditions. The invention further relates to pharmaceutical preparations and methods for use of such ligands in treatment and research. The invention further relates to pharmaceutical preparations provided with the aim of increasing presentation of MR1 ligands in clinical situations where such increased presentation of MR1 ligands is of clinical benefit.

### BACKGROUND OF THE INVENTION

MR1 (Uniprot ID 95460) is a non-polymorphic MHC class I-like protein that is expressed at low levels on the surface of most cell types. MR1 is highly conserved across multiple species, with human and mouse MR1 sharing >90% sequence homology at the protein level.

The inventors recently published their work confirming the existence of human T cells that recognize tumor-associated antigens (TAAs) presented by MR1 (Lepore et al., ELIFE 6, DOI:10.7554/eLife.24476). These novel T cells participate in tumor immune surveillance, thus representing novel tools for cancer immunotherapy. The antigens recognized by these MR1-specific T cells, however, remain unknown.

Adoptive therapy with donor- or patient-derived T cells engineered to express T-cell receptors (TCRs) specific for selected TAAs represents a promising and safe strategy to induce clinically relevant anti-tumor immune response in cancer patients. Targeting TAAs bound to MR1 non-polymorphic antigen presenting molecules might overcome this constraint and in principle be applicable to all patients bearing tumors expressing MR1. The use of tumor-reactive TCRs that recognize MR1-presented antigens might also have the advantage of complementing anti-tumor responses mediated by MHC-presented peptide antigens, excluding cross-competition of TAAs for binding to the same type of presenting molecule. In addition, this strategy may provide the possibility of targeting antigens of different nature on the same tumor cells, thus minimizing the potential occurrence of tumor escape variants under selective immune pressure.

The absence of information about the nature of the presented antigens, and the lack of tools available to probe, analyze and modulate the presentation of MR1, its interaction with antigen and with cognate TCRs, presents an obstacle in the development of improved MR1-centred immune-oncological treatments.

Therefore, the identification of MR1-presented TAAs and the identification and isolation of MR1-restricted TCRs recognizing these antigens might have important implications for cancer immunotherapy.

Based on the above-mentioned state of the art, the objective of the present invention is to provide means and methods to modulate MR1-TCR interaction to enable and improve clinical applications of MR1-based immunotherapy. This objective is attained by the subject-matter of the independent claims of the present specification.

### SUMMARY OF THE INVENTION

To the knowledge of the inventors, the present invention for the first time identifies MR1-specific eukaryotic antigen compounds presented to MR1 T cells in the context of an MR1-restricted immune response to intracellular antigens arising from metabolic states of the eukaryotic cell, thereby facilitating these compounds' uses for therapeutic/preventive and diagnostic or research purposes.

Consequently, the invention provides nucleobase-adduct MR1 ligand compounds, in many embodiments, nucleoside adducts or analogues, for use in methods harnessing the specificity of MR1-TCR interactions (with the MR1 molecule presenting the specific MR1 ligand compound to the T cell:
i) a method to stimulate MR1 ligand compound-specific T cells, and induce a prophylactic or therapeutic immune response,
ii) a method for the identification and isolation of T cells or antibodies reactive to the compounds as presented on MR1,
iii) a method to classify metabolically altered cells, including but not limited to tumor cells, according to the presence of the compounds.

When applied to a patient sample, the method to classify metabolically altered cells can be employed as a diagnostic method to stratify patients according to their likely response to MR1-specific T cell therapy, and may guide the administration of additional combination therapies such as MR1 ligand compounds as provided herein, or the administration of a pharmaceutical drug known to interfere with intracellular metabolism in a way that favours the generation of MR1 ligands as provided herein, or both.

MR1 ligand compounds for use in a method according to the invention are provided in claims 1 to 9 and Table 1.

The invention further relates to a method to modulate (increasing or decreasing) the quantity of MR1 ligand compounds in the cells or presented by the cells, by pharmacological intervention leading to accumulation or disposal of the MR1 ligand compounds in the cells.

In another aspect, the present invention relates a pharmaceutical composition comprising at least one of the MR1 ligand compounds of the present invention or a pharmaceutically acceptable salt thereof and at least one pharmaceutically acceptable carrier, diluent or excipient.

The invention further relates to TCR molecules capable of specifically recognizing an MR1 ligand as disclosed herein associated to (presented by) an MR1 molecule, as well as polynucleotide sequences encoding such MR1-ligand-specific TCR molecules.

The invention further provides 8-(9H-purin-6-yl)-2-oxa-8-azabicyclo[3.3.1]nona-3,6-diene-4,6-dicarbaldehyde, a novel MR1 ligand compound identified and synthesized for the first time herein.

### DETAILED DESCRIPTION OF THE INVENTION

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range.

Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

The term MR1 in the context of the present specification refers to either the MR1 gene (Entrez 3140) or the MR1 gene product (Uniprot Q95460), also referred to herein as “MR1 polypeptide” or “MR1 molecule”. By way of non-limiting example, an MR1 polypeptide may be present in an aspect or embodiment disclosed herein as an isolated MR1 polypeptide, for example in the context of an MR1 polypeptide tetramer (Gherardin, Immunol Cell Biol. 2018 May; 96(5):507-525) or expressed on a patient's cells either naturally or in response to the transfer of an MR1-encoding gene construct.

The term MR1T cell in the context of the present specification refers to a T cell that expresses a T cell receptor capable of binding specifically to an MR1 molecule presenting an antigen molecule as specified herein.

The term MR1T cell receptor in the context of the present specification refers to a T cell receptor capable of binding specifically to an antigen presented, for example by a cancer cell, in association with an MR1 molecule.

The expression of a marker such as MR1 may be assayed via techniques such as fluorescence microscopy, flow cytometry, ELISPOT, ELISA or multiplex analyses.

A TCR sequence or TCR molecule described herein comprises, to be fully functional, a TCR alpha and a TCR beta polypeptide chain, or a TCR gamma and a TCR delta polypeptide chain. If reference is made to a TCR alpha or beta polypeptide having a particular sequence, it is understood that in order for this to be fully functional in the methods and cells described herein, it requires the presence of a complementary (beta or alpha, respectively) polypeptide chain. The same applies, mutatis mutandis, to the gamma delta pairing. Mention of a specific TCR alpha, beta, gamma or delta sequence implies the possibility that it is paired with the TCR sequence with which it is paired in the original clone as described herein, or a sequence of certain identity to the original pairing sequence, as specified herein. Mention of a specific TCR alpha, beta, gamma or delta sequence also implies the possibility that it is paired with another pairing TCR sequence.

The recognition of MR1-presented cancer antigens is effected mainly through CDR3 sequences. Wherein a TCR sequence characterized only by a specific CDR3 sequence is mentioned herein, it is implied that the TCR sequence is a full alpha, beta, gamma or delta TCR sequence as provided herein, and a resulting TCR molecule is paired with an appropriate second sequence.

The term compound in association with MR1 in the context of the present specification refers to a compound which is non-covalently bound by an MR1 molecule. Binding may occur, for example, via Van-der-Waals forces and electrostatic interactions, including hydrogen bonds.

The compound and the MR1 molecule form a complex, which may be recognized by a specific T cell receptor. Recognition by a specific T cell receptor means that the T cell receptor can differentiate between an MR1 molecule without association with the compound, and the MR1-ligand compound-MR1complex.

In the context of the present specification, the terms sequence identity and percentage of sequence identity refer to a single quantitative parameter representing the result of a sequence comparison determined by comparing two aligned sequences position by position. Methods for alignment of sequences for comparison are well-known in the art. Alignment of sequences for comparison may be conducted by the local homology algorithm of Smith and Waterman, Adv. Appl. Math. 2:482 (1981), by the global alignment algorithm of Needleman and Wunsch, J. Mol. Biol. 48:443 (1970), by the search for similarity method of Pearson and Lipman, Proc. Nat. Acad. Sci. 85:2444 (1988) or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.ncbi.nlm.nih.gov/).

One example for comparison of amino acid sequences is the BLASTP algorithm that uses the default settings: Expect threshold: 10; Word size: 3; Max matches in a query range: 0; Matrix: BLOSUM62; Gap Costs: Existence 11, Extension 1; Compositional adjustments: Conditional compositional score matrix adjustment. One such example for comparison of nucleic acid sequences is the BLASTN algorithm that uses the default settings: Expect threshold: 10; Word size: 28; Max matches in a query range: 0; Match/Mismatch Scores: 1.-2; Gap costs: Linear. Unless stated otherwise, sequence identity values provided herein refer to the value obtained using the BLAST suite of programs (Altschul et al., J. Mol. Biol. 215:403-410 (1990)) using the above identified default parameters for protein and nucleic acid comparison, respectively.

Reference to identical sequences without specification of a percentage value implies 100% identical sequences (i.e. the same sequence).

In the present specification, the term positive, when used in the context of expression of a marker, refers to expression of an antigen assayed by a fluorescent labelled antibody, wherein the fluorescence is at least 5% higher (>5%), in median fluorescence intensity in comparison to staining with an isotype-matched antibody which does not specifically bind the same target. Such expression of a marker is indicated by a superscript “plus” (+), following the name of the marker, e.g. MR1+.

In the present specification, the term negative, when used in the context of expression of a marker, refers to expression of an antigen assayed by a fluorescent labelled antibody, wherein the median fluorescence intensity is less than 5% higher than the median fluorescence intensity of an isotype-matched antibody which does not specifically bind the same target. Such expression of a marker is indicated by a superscript minus (−), following the name of the marker, e.g. MR1.

In the context of the present specification, the term checkpoint modulator agent encompasses checkpoint inhibitory agents (particularly checkpoint inhibitory antibodies) and checkpoint agonist agents (particularly checkpoint agonist antibodies).

The term having substantially the same biological activity in the context of the present specification relates, when used to define a TCR molecule capable of recognizing an MR1 ligand bound to an MR1 molecule, to the capability to recognize (or contribute in the recognition of) its cognate ligand (the MR1 ligand associated with MR1). Assays and methods to determine such interaction are described herein.

The term nucleic acid expression vector in the context of the present specification relates to a polynucleotide, for example a plasmid, a viral genome or a synthetic RNA molecule, which is used to transfect (in case of a plasmid or an RNA) or transduce (in case of a viral genome) a target cell with a certain gene of interest. In the case of a DNA expression construct, the gene of interest is under control of a promoter sequence and the promoter sequence is operational inside the target cell, thus, the gene of interest is transcribed either constitutively or in response to a stimulus or dependent on the cell's status. In the case of an RNA expression construct, it is understood that the term relates to translation of the RNA and the construct can be employed by the target cell as an m-RNA. In certain embodiments, the viral genome is packaged into a capsid to become a viral vector, which is able to transduce the target cell.

The term transgenic MR1-reactive T cell in the context of the present invention relates to an autologous or allogeneic T cell expressing a T cell receptor (TCR) that specifically recognizes an MR1 molecule expressed on a patient's cell. In certain embodiments, the TCR recognizes MR1-expressing tumour cells in the absence of any added foreign antigen and in MR1-dependent manner. MR-1 restricted TCR sequences are disclosed in PCT/EP2019/074284 and US 20190389926 A1, both of which are incorporated herein by reference.

In the context of the present specification, the term checkpoint inhibitory agent or checkpoint inhibitory antibody is meant to encompass an agent, particularly an antibody (or antibody-like molecule) capable of disrupting the signal cascade leading to T cell inhibition after T cell activation as part of what is known in the art the immune checkpoint mechanism. Non-limiting examples of a checkpoint inhibitory agent or checkpoint inhibitory antibody include antibodies to CTLA-4 (Uniprot P16410), PD-1 (Uniprot Q15116), TMIGD2 (Uniprot Q96BF3), BTLA (Uniprot Q7Z6A9), CD160 (Uniprot O95971), Lag-3 (Uniprot P18627), TIGIT (Uniprot Q495A1), CD96 (Uniprot P40200), TIM-3 (Uniprot Q8TDQ0), CEACAM1 (Uniprot P13688), SIRP alpha (Uniprot P78324), CD200R (Uniprot Q8TD46), KIR family (including Uniprot proteins Q99706, P43628, P43626, Q8NHK3, P43627, Q8N109, B0L652, Q86U48, B0L653, A0A191URI1, Q6H2H3), ILT family, or to PD-L1 (Uniprot Q9NZQ7), PD-L2 (Uniprot Q9BQ51), VISTA (Uniprot Q9H7M9), B7H3 (CD276; Uniprot Q5ZPR3), CD80 (Uniprot P33681), CD86 (Uniprot P42081), B7-H4 (Uniprot P42081), TNFRSF14 (HVEM, CD270, Uniprot Q92956), CD155 (Uniprot P15151), Galectin9 (Uniprot O00182), CD200 (Uniprot Q8TD46).

In the context of the present specification, the term checkpoint agonist agent or checkpoint agonist antibody is meant to encompass an agent, particularly but not limited to an antibody (or antibody-like molecule) capable of engaging the signal cascade leading to T cell activation as part of what is known in the art the immune checkpoint mechanism. Non-limiting examples of receptors known to become upregulated upon T cell activation include CD25 (Uniprot P01589), CD122 (Uniprot P14784) and CD137 (4-1BB; Uniprot Q07011). The term checkpoint agonist agent or checkpoint agonist antibody encompasses agonist antibodies to CD28 (P10747), ICOS (Q9Y6W8), CD137 (4-1BB; Uniprot Q07011), Light (HVEM, Uniprot O43557), OX40 (P23510), GITR (Q9Y5U5), DNAM-1 (CD226, Uniprot Q15762), 2B4 (CD244, Uniprot Q9BZW8), DR3 (Q93038), NKp80 (KLRF1, Uniprot Q9NZS2).

The term C₁-C₄ alkyl in the context of the present specification relates to a saturated linear or branched hydrocarbon having 1, 2, 3 or 4 carbon atoms, wherein in certain embodiments one carbon-carbon bond may be unsaturated and one CH₂ moiety may be exchanged for oxygen (ether bridge) or nitrogen (NH, or NR with R being methyl, ethyl, or propyl; amino bridge). Non-limiting examples for a C₁-C₄ alkyl are methyl, ethyl, propyl, prop-2-enyl, n-butyl, 2-methylpropyl, tert-butyl, but-3-enyl, prop-2-inyl and but-3-inyl. In certain embodiments, a C₁-C₄ alkyl is a methyl, ethyl, propyl or butyl moiety.

The term unsubstituted Cₙ alkyl when used herein in the narrowest sense relates to the moiety —CₙH₂ₙ— if used as a bridge between moieties of the molecule, or —CₙH₂ₙ₊₁ if used in the context of a terminal moiety.

The terms unsubstituted Cₙ alkyl and substituted Cₙ alkyl include a linear alkyl comprising or being linked to a cyclical structure, for example a cyclopropane, cyclobutane, cyclopentane or cyclohexane moiety, unsubstituted or substituted depending on the annotation or the context of mention, having linear alkyl substitutions. The total number of carbon and—where appropriate—N, O or other hetero atom in the linear chain or cyclical structure adds up to n.

The term substituted alkyl in its broadest sense refers to an alkyl as defined above in the broadest sense, which is covalently linked to an atom that is not carbon or hydrogen, particularly to an atom selected from N, O, F, B, Si, P, S, Cl, Br and I, which itself may be—if applicable-linked to one or several other atoms of this group, or to hydrogen, or to an unsaturated or saturated hydrocarbon (alkyl or aryl in their broadest sense). In a narrower sense, substituted alkyl refers to an alkyl as defined above in the broadest sense that is substituted in one or several carbon atoms by groups selected from amine NH₂, alkylamine NHR, imide NH, alkylimide NR, amino(carboxyalkyl) NHCOR or NRCOR, hydroxyl OH, oxyalkyl OR, oxy(carboxyalkyl) OCOR, carbonyl O and its ketal or acetal (OR)₂, nitril CN, isonitril NC, cyanate CNO, isocyanate NCO, thiocyanate CNS, isothiocyanate NCS, fluoride F, chloride Cl, bromide Br, iodide I, phosphonate PO₃H₂, PO₃R₂, phosphate OPO₃H₂ and OPO₃R₂, sulfhydryl SH, suflalkyl SR, sulfoxide SOR, sulfonyl SO₂R, sulfanylamide SO₂NHR, sulfate SO₃H and sulfate ester SO₃R, wherein the R substituent as used in the current paragraph, different from other uses assigned to R in the body of the specification, is itself an unsubstituted or substituted C₁ to C₁₂ alkyl in its broadest sense, and in a narrower sense, R is methyl, ethyl or propyl unless otherwise specified.

The term amino substituted alkyl or hydroxyl substituted alkyl refers to an alkyl according to the above definition that is modified by one or several amine or hydroxyl groups NH₂, NHR, NR2 or OH, wherein the R substituent as used in the current paragraph, different from other uses assigned to R in the body of the specification, is itself an unsubstituted or substituted C₁ to C₁₂ alkyl in its broadest sense, and in a narrower sense, R is methyl, ethyl or propyl unless otherwise specified. An alkyl having more than one carbon may comprise more than one amine or hydroxyl.

Unless otherwise specified, the term “substituted alkyl” refers to alkyl in which each C is only substituted by at most one amine or hydroxyl group, in addition to bonds to the alkyl chain, terminal methyl, or hydrogen.

The term carboxyl substituted alkyl refers to an alkyl according to the above definition that is modified by one or several carboxyl groups COOH, or derivatives thereof, particularly carboxylamides CONH₂, CONHR and CONR₂, or carboxylic esters COOR, with R having the meaning as laid out in the preceding paragraph and different from other meanings assigned to R in the body of this specification.

The term halogen-substituted alkyl refers to an alkyl according to the above definition that is modified by one or several halogen atoms selected (independently) from F, Cl, Br, I.

A carboxylic ester is a group —CO₂R, with R being defined further in the description. A carboxylic amide is a group —CONHR, with R being defined further in the description.

As used herein, the term pharmaceutical composition refers to a compound of the invention, or a pharmaceutically acceptable salt thereof, together with at least one pharmaceutically acceptable carrier. In certain embodiments, the pharmaceutical composition according to the invention is provided in a form suitable for topical, parenteral or injectable administration.

As used herein, the term pharmaceutically acceptable carrier includes any solvents, dispersion media, coatings, surfactants, antioxidants, preservatives (for example, antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington: the Science and Practice of Pharmacy, ISBN 0857110624).

As used herein, the term treating or treatment of any disease or disorder (e.g. cancer) refers in one embodiment, to ameliorating the disease or disorder (e.g. slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. Methods for assessing treatment and/or prevention of disease are generally known in the art, unless specifically described hereinbelow.

In the present specification, the following abbreviations are used: APC, antigen-presenting cell; β2m, β2 microglobulin; DC, dendritic cell; HLA, human leukocyte antigen; HPLC, high-pressure liquid chromatography; IFN-γ, interferon-γ; IL-13, interleukin 13; mAb, monoclonal antibody; MAIT cell, mucosal associated invariant T cell; MFI, median fluorescence intensity; MHC, major histocompatibility complex; MR1, MHC class I-related molecule 1; MR1T cell, MR1-restricted T cell; MS, mass-spectrometry; PBMC, peripheral blood mononuclear cell; reactive oxygen species, ROS; TAA, tumor-associated antigen; TCR, T cell receptor; TIL, tumor-infiltrating lymphocyte.

In certain aspects, the invention relates to the use of an MR1 (major histocompatibility complex class I-related gene protein 1) ligand compound in a method comprising the modulation of the interaction of MR1 with an MR1 specific T cell.

Certain MR1 ligand compounds according to the invention can be described by an adenine derivative of the following general formulas:

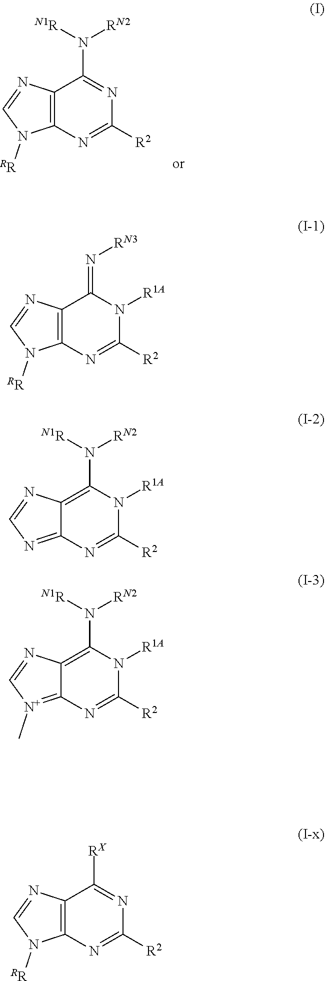

Other MR1 ligand compounds to the invention can be described by a guanine derivative of the following general formulas:

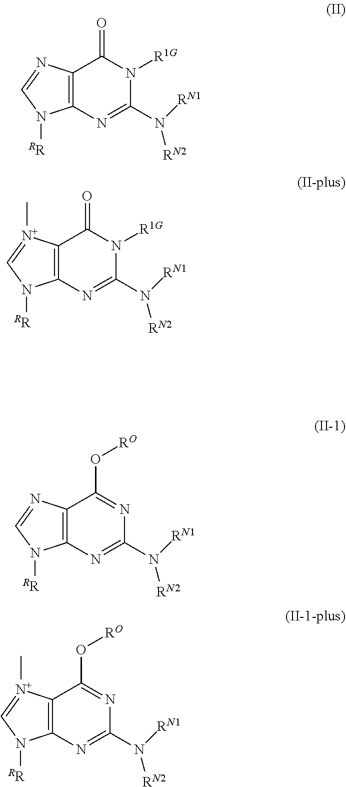

Other MR1 ligand compounds according to the invention can be described by a pyrimidine nucleotide derivative of the following general formulas:

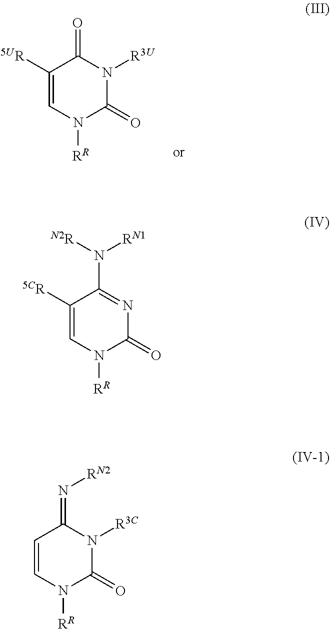

The substituents indicated in the above general formulas can assume the following meanings:
R^{1A} is H or methyl, R^{1G} is H or methyl;
R^{N3} is H or methyl;
R² is selected from H, methyl and —S-methyl;
R^{3U} and R^{5U} are selected from H and methyl; R^{5C} is selected from H and methyl;
R^{N1} and R^{N2} are both H or C₁ to C₃alkyl, or R^{N1} is H or C₁ to C₃ alkyl (particularly R^{N1} is H or methyl) and R^{N2} is selected from a C₁-C₆ alkyl and a C₂-C₆ alkylene and a C₁-C₆ alkyl substituted carbamoyl, wherein the alkyl or alkylene is unsubstituted or substituted with carbonyl, carboxyl and/or hydroxyl, particularly R^{N1} is H and R^{N2} is selected from a methyl, 2-hydroxy-ethyl, 1-carboxethyl, 1,2-dicarboxy-ethyl, threonylcarbamoyl, isopent-2-enyl, cis-hydroxyisopent-2-enyl, 3-oxo-1-propenyl, and hexa-1,3,5-triene-1,1,3-tricarbaldehyde;
or R^{N1}, R^{N2} and the nitrogen together form a 2-oxa-8-azabicyclo [3.3.1] nona-3,6-diene-4,6-dicarbaldehyde bi-annular system;
or
R^{N3} and R^{1A} together, or R^{N2} and R^{3C} together, or R^{N1} and R^{1G} together form an unsubstituted or C₄ to C₁₆-2-oxo-alkyl-substituted or ω-carboxy-2-oxo-alkyl-substituted imidazole ring, or
R^{N3} and R^{1A} together, or R^{N2} and R^{3C} together, or R^{N1} and R^{1G} together are —C(CH₃)OH—CHOH— or C(R′)OH— CH₂—CHOH— or oxy-cyclopropylidene-malonaldehyde-substituted prop-2-ene with R′ being selected from H, CH₃, CH(OH)C₂H₅, C₂H₅ and C₄H₉;
or R^{N1} and R^{1G} form a pyrimidine, or R^{N1} and R^{1G} or R^{N3} and R^{1A} form a 12-oxo-5,6,10,12-tetrahydro-3H-6,10-methano[1,3,5]oxadiazocine ring system, or R^{N1} and R^{1G} form a 2-oxa-6,8-diazabicyclo [3.3.1] nona-3-ene-4-carbaldehyde bi-annular system;
and
R^{O} is selected from H, unsubstituted or hydroxyl-substituted C₁-C₅ alkyl or C₂-C₅ alkylene,
R^{X} is selected from SH, C₁-C₅ alkyl, C₂-C₅ alkylene, and C₁-C₅ S-alkyl;
R^{R} is selected from H, 1′-ribosyl, 2′-deoxy-1′-ribosyl, 5′-phospho-1′-ribosyl, 5′-methylthio-1′-ribosyl, 1′-(2′-O-ribosyl-5″-phosphate)ribosyl, 1′-(2′-O-ribosyl)-ribosyl 1′-(2′-O-methyl)ribosyl.

The inventors' present understanding is that the “modification of the nucleoside” represented by moieties R^{1A}, R², R^{N1}, R^{N2}, R^{N3}, etc. that constitute the structural difference between the adduct and the parent nucleoside, facilitates interactions with the MR1 pocket, thus generating a stable complex with MR1. No structural information has been obtained yet to provide a rationale of how the different adducts are inserted within the MR1 pocket. Different adducts might assume orthogonal positions. For example, while adenosine-containing adducts might have the two rings parallel to the alfa helices of MR1, the guanosine adducts might position the two rings in a perpendicular manner. This might explain while addition of residues in the two different positions of the primary amine of adenosine and guanosine are both compatible with binding. Data obtained from crystal structures of MR1 binding bacterial MR1 ligands support this notion.

The natural nucleobases and their ribosyl or deoxyribosyl derivatives, i.e. adenine, adenosine, deoxyadenosine, guanine, guanosine, deoxyguanosine, uracil, uridine, deoxyuridine, thymine, thymidine, deoxythymidine, cytosine and cytidine and deoxycytidine are not deemed to be encompassed by the scope of general formulas I, II, III and IV and their derivatives contemplated above, and are not deemed useful for the methods and uses disclosed herein.

Also, the inventors have found that the compounds 3-methyladenine, 7-methyl-7-deaza-2′-deoxyguanosine, queuosine, wybutosine, hydroxywybutosine, pseudouridine, and (2R,3S,4R,5R)-2-(hydroxymethyl)-5-(6-(methylthio)-9H-purin-9-yl)tetrahydrofuran-3,4-diol can be used for the practice of the invention, as can be modifications thereof formed in the spirit of the invention as disclosed herein.

In certain embodiments of any of the nucleoside derivative compounds disclosed herein as MR1 ligands, R^{R} is described by the general formula (V)

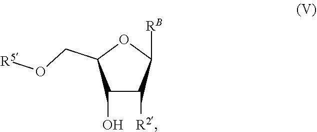

wherein R^{B} is the bond connecting the moiety to the N⁹ nitrogen of the group I formula (adenine nucleobase derivatives) identified above, or to the N¹ nitrogen of the group II, III or IV formulas above.

In certain embodiments, R^{2′} of V is H and R^{5′} is H. In certain embodiments, R^{2′} of V is H and R^{5′} is PO₃²⁻. In certain embodiments, R^{2′} of V is H and R^{5′} is OH. In certain embodiments, R^{2′} of V is OH and R^{5′} is PO₃²⁻. In certain embodiments, R^{2′} of V is OCH₃ and R⁵ is H. In certain embodiments, R^{2′} of V is OCH₃ and R^{5′} is PO₃²⁻.

In certain embodiments, R^{2′} of V is O-ribosyl or O-ribosyl-5″-phosphate, and R^{5′} is selected from H and PO₃²⁻.

In certain particular embodiments of any of the nucleoside derivative compounds disclosed herein as MR1 ligands, R^{R} is described by the general formula (Va)

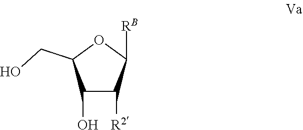

wherein R^{2′} is selected from H, OH, O-methyl, O-1-ribosyl and O-1-(5-phospho)-ribosyl. Va with R^{2′} being OH is shown as Vb; Va with R^{2′} being O-1-(5-phospho)-ribosyl is shown as Vc.

In certain more particular embodiments of any of the nucleoside derivative compounds disclosed herein as MR1 ligands, R^{R} is described by the general formula (Vb) or (Vc)

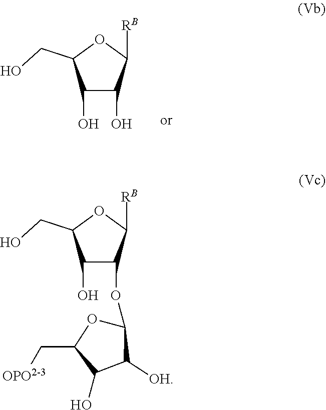

It is understood that the phosphate on the 5″ ribosyl oxygen of Vc can be in acid form (OPO₃H₂) or as a hydrogen phosphate or phosphate salt together with a suitable anion.

In certain embodiments of any of the nucleoside derivative compounds disclosed herein as MR1 ligands, the MR1 ligand compound is described by any one of the following general formulas

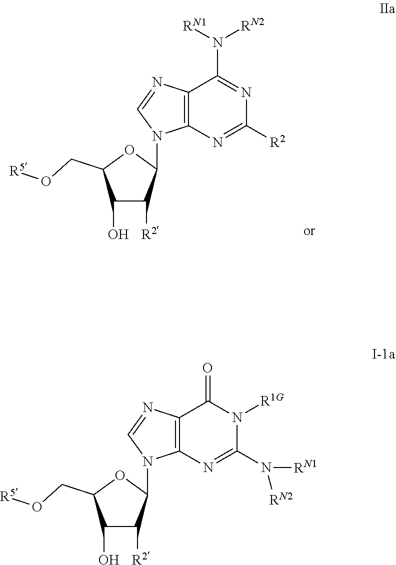

wherein R^{N1}, R^{N2}, R^{1A}, R^{1G}, R^{2′} and R^{5′} can have the meaning indicated above,
and wherein R^{2′} is selected from H, OH, O-methyl, ribosyl and 5″phosphoribosyl, and R^{5′} is selected from H, PO₃²— and methyl,
particularly R^{2′} is H or OH, and R^{5′} is H.

In certain particular embodiments, the MR1 ligand compound is described by formula I and R^{N1}, R^{N2} and the nitrogen together form a 2-oxa-8-azabicyclo [3.3.1] nona-3,6-diene-4,6-dicarbaldehyde bi-annular system, and R is selected from H, ribosyl and deoxyribosyl.

In certain embodiments of any of the nucleoside derivative compounds disclosed herein as MR1 ligands, the MR1 ligand compound is described by
a. formula (I) wherein R² is S-methyl and R^{N1} and R^{N2} are both H;
b. formula (I) wherein R² is methyl and R^{N1} and R^{N2} are both H; or
c. formula (I-1) wherein R^{1A} is methyl, R² is H and R^{N3} is H; or
d. formula (I-1) wherein R^{1A} is methyl, R² is methyl and R^{N3} is H; or
e. formula (I-1) wherein R^{1A} is methyl, R² is S-methyl and R^{N3} is H; or
f. formula (I) wherein R² is H, one of R^{N1} and R^{N2} is selected from H and methyl, and the other one of R^{N1} and R^{N2} is selected from methyl, 3-methylbut(2)enyl, 3-hydroxymethylbut(2)enyl and threonylcarbamoyl; or
g. formula (I) wherein R² is S-methyl and one of R^{N1} and R^{N2} is selected from H and methyl, and the other one of R^{N1} and R^{N2} is selected from methyl, ethan-2-ol, 1,2-dicarboxy-ethyl, 3-methylbut(2)enyl, 3-hydroxymethylbut(2)enyl and threonylcarbamoyl;
h. formula (I) wherein R² is methyl and one of R^{N1} and R^{N2} is selected from H and methyl, and the other one of R^{N1} and R^{N2} is selected from methyl, 3-methylbut(2)enyl, 3-hydroxymethylbut(2)enyl and threonylcarbarnoyl;
i. formula (II) wherein R^{1G} is methyl, and R^{N1} and R^{N2} are both H; or
j. formula (II-plus) wherein R^{1G} is methyl, and R^{N1} and R^{N2} are both H; or
k. formula (II-plus) wherein R^{1G} is H, and R^{N1} and R^{N2} are both H; or
l. formula (II) wherein R^{1G} is methyl, and one of R^{N1} and R^{N2} is selected from H and methyl, and the other one of R^{N1} and R^{N2} is selected from methyl, ethyl, 3-methylbut(2)enyl, 3-hydroxymethylbut(2)enyl and threonylcarbarnoyl; or
m. formula (II-plus) wherein R^{1G} is methyl, and one of R^{N1} and R^{N2} is selected from H and methyl, and the other one of R^{N1} and R^{N2} is selected from methyl, 3-methylbut(2)enyl, 3-hydroxymethylbut(2)enyl and threonylcarbarnoyl; or
n. formula (II-plus) wherein R^{1G} is H, and one of R^{N1} and R^{N2} is selected from H and methyl, and the other one of R^{N1} and R^{N2} is selected from methyl, 3-methylbut(2)enyl, 3-hydroxymethylbut(2)enyl and threonylcarbarnoyl; or
o. formulas (I-IM or II-IM), with R^{IM} selected from H, CH₂COCₙH₍₂ₙ₊₁₎ with n from 3 to 7 (particularly n=5), and CH₂CO(CH₂)ₘCOO⁻ with m from 3 to 9 (particularly n=7)

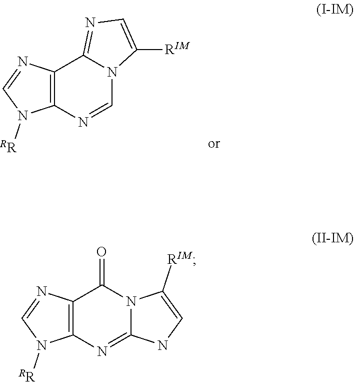

p. formulas (II-c), (II-d), (II-e) (II-f)

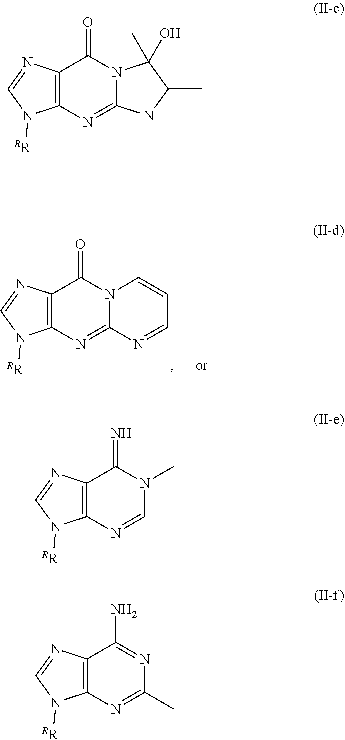

q. formulas (II-g) or (II-h), wherein R² is selected from H, methyl and S-methyl:

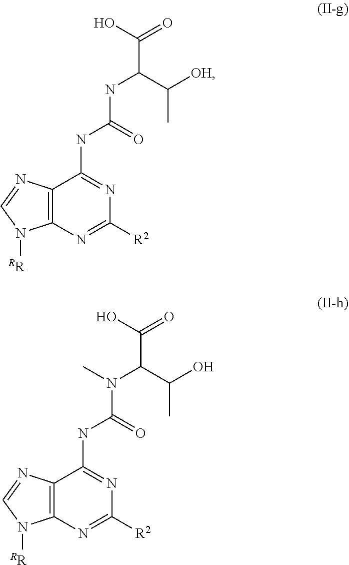

r. formulas (II-i) (II-j), wherein R^{N1} is selected from H and methyl and wherein R² is selected from H, methyl and S-methyl:

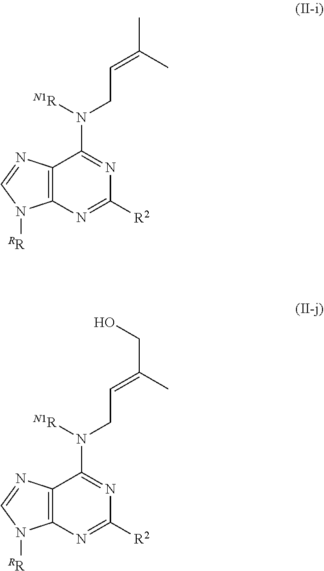

s. formula (III), wherein R^{3U} is H and R^{5U} is methyl,
t. formula (III), wherein Rau is methyl and R^{5U} is H,
u. formula (III), wherein R^{3U} and R^{5U} are both methyl,
v. formula (IV), wherein R^{5C} is H, one of R^{N1} and R^{N2} is selected from H and methyl, and the other one of R^{N1} and R^{N2} is selected from methyl, 3-methylbut(2)enyl, 3-hydroxymethylbut(2)enyl and threonylcarbarnoyl;
w. formula (IV-1), wherein R^{N2} is selected from methyl, 3-methylbut(2)enyl, 3-hydroxymethylbut(2)enyl and threonylcarbarnoyl; or
x. formula (IV), wherein R^{5C} is methyl, one of R^{N1} and R^{N2} is selected from H and methyl, and the other one of R^{N1} and R^{N2} is selected from methyl, 3-methylbut(2)enyl, 3-hydroxymethylbut(2)enyl and threonylcarbarnoyl;
y. formula (Ix), wherein R² is H, and R^{X} is selected from methyl and S-methyl;
z. formula (I), wherein R² is H, R^{N1} is H, R^{N2} is 3-oxo-1-propenyl;
aa. formula (I-a), (I-a)

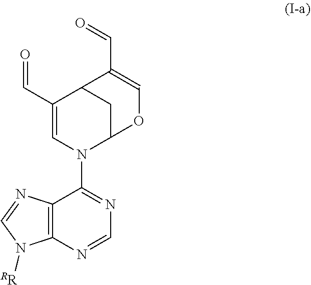

bb. formula (II-k), (II-k)

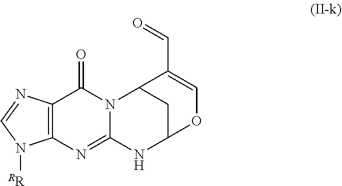

cc. formula (I-2), wherein R^{1A}, R^{N1} and R^{2′} are H, and R^{N2} is isopent-2-enyl, or cis-hydroxyisopent-2-enyl,
dd. formula (I), wherein R^{N1} and R² are both H, and R^{N2} is isopent-2-enyl, or cis-hydroxyisopent-2-enyl,
ee. formula (II-1), wherein R^{O} is methyl or ethan-2-ol, and R^{N1} and R² are both H;
ff. formula (I-b),

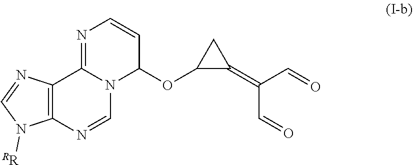

and wherein in each formula, R^{R} is selected from H, 1′-ribosyl, 2′-deoxy-1′-ribosyl, 5′-phospho-1′-ribosyl, 5′-methylthio-1′-ribosyl, 1′-(2′-O-ribosyl-5″-phosphate)ribosyl, 1′-(2′-O-ribosyl)-ribosyl 1′-(2′-O-methyl)ribosyl.

In certain particular embodiments of the above embodiments outlined as a., b., . . . ff., gg., R^{R} is H.

In certain particular embodiments of the above embodiments outlined as a., b., . . . ff., gg., R^{R} is (1) ribosyl (Vb).

In certain particular embodiments of the above embodiments outlined as a., b., . . . ff., gg., R^{R} is (1) desoxyribosyl (Vd).

MR1 ligand compounds defined by full structure:

In certain very particular embodiments of any of the nucleoside derivative compounds disclosed herein as MR1 ligands, the MR1 ligand compound is selected from
a. 1-methyladenosine (1)
b. 2-methyladenosine (2)
c. 2′-O-methyladenosine (3)
d. N6,N6-dimethyladenosine (4)
e. N6-threonylcarbamoyladenosine (5)
f. N6-isopent-2-enyladenosine (6)
g. N6-(cis-hydroxyisopent-2-enyl) adenosine (7)
h. 2-methylthio-N6-(cis-hydroxyisopent-2-enyl) adenosine (8)
i. 2-methylthio-N6-isopent-2-enyladenosine (9)
j. N6-methyl-N6-threonylcarbamoyladenosine (10)
k. 2′-O-ribosyladenosinephosphate (11)
l. N6-(3-oxo-1-propenyl)-2′-deoxyadenosine (12)
m. 8-(9H-purin-6-yl)-2-oxa-8-azabicyclo[3.3.1]nona-3,6-diene-4,6-dicarbaldehyde (13)
n. 1-(3-((2R,4S,5R)-4-hydroxy-5-(hydroxymethyl) tetrahydrofuran-2-yl)-3H-imidazo[2,1-4]purin-7-yl) heptan-2-one (14)
o. 1-methylguanosine (15)
p. N2-methylguanosine (16)
q. 7-methylguanosine (17)
r. 2′-O-methylguanosine (18)
s. N2,N2-dimethylguanosine (19)
t. 2′-O-ribosylguanosine (20)
u. 3-((2R,3R,4S,5R)-3,4-dihydroxy-5-(hydroxymethyl)tetrahydrofuran-2-yl)-6,7-dihydroxy-6-methyl-6,7-dihydro-3H-imidazo[1,2-a]purin-9(5H)-one (21), 3-((2R,3R,4S,5R)-3,4-dihydroxy-5-(hydroxymethyl)tetrahydrofuran-2-yl)-6,7-dihydroxy-7-methyl-6,7-dihydro-3H-imidazo[1,2-a]purin-9(5H)-one (22), or a mixture of the two;
v. 2-((6-oxo-6,7-dihydro-1H-purin-2-yl)amino)propanoate (23)
w. pyrimido[1,2-a]purin-10(3H)-one (M1G) (24).
x. 3-((2R,5R)-4-hydroxy-5-(hydroxymethyl) tetrahydrofuran-2-yl)-7-(2-oxoheptyl)-3H-imidazo[1,2-a]purin-9(5H)-one (25)
y. 3-(2-deoxy-β-D-erythro-pentofuranosyl)pyrimido[1,2-a]purin-10(3H)-one (26)
z. N2-oxopropenyl-deoxyguanosine (27)
aa. 3-((2R,4S,5R)-4-hydroxy-5-(hydroxymethyl)tetrahydrofuran-2-yl)-12-oxo-5,6,10,12-tetrahydro-3H-6,10-methano[1,3,5]oxadiazocino[5,4-a]purine-9-carbaldehyde (28)
bb. 2′-O-methylcytidine (29)
cc. 3-methyluridine (30)
dd. 5-methyluridine (31)
ee. 3,2′-O-dimethyluridine (32)
ff. 6-((2R,4S,5R)-4-hydroxy-5-(hydroxymethyl)tetrahydrofuran-2-yl)-3-(2-oxoheptyl)-1,8a-dihydroimidazo[1,2-c]pyrimidin-5(6H)-one (37)
gg. N4-(3-oxo-1-propenyl)-2′-deoxycytidine (38)
hh. 8-(1-((2R,4S,5R)-4-hydroxy-5-(hydroxymethyl)tetrahydrofuran-2-yl)-2-oxo-1,2-dihydropyrimidin-4-yl)-2-oxa-8-azabicyclo[3.3.1]nona-3,6-diene-4,6 dicarbaldehyde (39)
ii. 3-methyladenine (41)
jj. N6-methyladenosine (42)
kk. 6-methylpurine (43)
ll. 6-(dimethylamino)purine (44)
mm. N6-(A2-isopentenyl) adenine (45)
nn. N1-methyl-2′-deoxyguanosine (46)
oo. 1-methylguanine (47)
pp. N2-methyl-2′-deoxyguanosine (48)
qq. 7-methyl-7-deaza-2′-deoxyguanosine (49)
rr. O6-methyl-2′-deoxyguanosine (50)
ss. N2-ethyl-2′-deoxyguanosine (51)
tt. 5′-deoxy-5′-(methylthio)adenosine (52)
uu. N6-methyl-2′-deoxyadenosine (53)
vv. N6-(2-hydroxyethyl)-2′-deoxyadenosine (54)
ww. 06-(2-hydroxyethyl)-2′-deoxyguanosine (55)
xx. N6-succinyl adenosine (56)
yy. 2-(2-((3-((2R,4S,5R)-4-hydroxy-5-(hydroxymethyl)tetrahydrofuran-2-yl)-3,7-dihydropyrimido[2,1-i]purin-7-yl)oxy)cyclopropylidene)malonaldehyde (57);

The invention further encompasses variations of the modifications shown herein that can be obtained by modifying the moieties attached to the nucleoside core structure by adding amine functions, hydroxy functions, carboxylic acid or carboxylic acid ester moieties or halogens. Any of the compounds showing an alkyl moiety above may be modified to show an aminoalkyl, hydroxyalkyl or haloalkyl as defined above. Mixed modifications are possible. The assays shown herein provide a solid basis for testing whether such modifications are capable of binding to MR1 and of triggering or inhibiting a MR1T cell response.

In a general aspect, the invention relates to a method for modulating, particularly in vitro, an interaction between an MR1 polypeptide and an MR1-specific TCR molecule. The method according to this aspect comprises contacting an MR1 polypeptide with an MR1 ligand compound as specified above.

### Methods to Select T Cells, TCR, B Cells or Antibodies Reactive to MR1-Presented MR1 Ligands

In one set of embodiments, a method of modulating the MR1-ligand interaction can be practiced in-vitro, for example to search for and identify novel binders, particularly T cells, B cells or antibodies reactive to the MR1-ligand complex. Such binding molecules, or polynucleotide sequences encoding them, can subsequently be developed as pharmaceutical agents or diagnostic reagents, or a particularly useful reagent can be selected from a pre-existing repertoire.

These sequences then provide access to a variety of tools that will lend themselves to their use in pharmaceutical and diagnostic applications.

For example, an MR-1 presenting cell is contacted with a ligand as disclosed herein in the presence of a T cell library or a B cell library (cells expressing a variety of TCR or B cell receptor (BCR) sequences), and cells bearing TCR or BCR sequences reactive to the MR1-ligand complex are identified and isolated by methods known in the art.

Accordingly, one aspect of the invention relates to a method for identification, isolation or selection of a T cell reactive to a MR1 ligand compound as specified herein, particularly in any one of claims 1 to 9, as presented on MR1. This method comprises the steps of providing a preparation of T cells reactive to/capable of specifically recognizing MR1; contacting said preparation of T cells with a complex comprising isolated MR1, or an MR1 on an MR1 presenting cell, associated to said MR1 ligand compound and then isolating a T cell that is specifically reactive to said MR1 ligand compound in the context of MR1 presentation, in an isolation step. A number of ways are available to the skilled person to detect TCR engagement with and recognition of a cognate antigen presented by MHC.

According to an alternative of this aspect of the invention, the method for modulating an interaction between an MR1 polypeptide and an MR1-specific TCR molecule can be employed as part of a method for the identification and isolation of B cells or their receptors, antibodies, specific for MR1 polypeptide and/or reactive to the MR1 ligand compound as presented on MR1.

An alternative of this aspect of the invention relates to a method for identification, isolation or selection of a B cell or antibody reactive to a MR1 ligand compound as specified herein, particularly in any one of claims 1 to 9. This method comprises the steps of providing a preparation of B cells reactive to/capable of specifically recognizing MR1; contacting said preparation of B cells with a complex comprising isolated MR1 associated to said MR1 ligand compound and then isolating a B cell that is specifically reactive to said MR1 ligand compound in the context of MR1 presentation, in an isolation step. A number of ways are available to the skilled person to detect BCR engagement with and recognition of a cognate antigen presented by MHC.

According to another aspect of the invention, the method for modulating an interaction between an MR1 polypeptide and an MR1-specific TCR molecule can be employed as part of a method of diagnosis to classify metabolically altered cells, including but not limited to tumor cells, according to the presence of the compounds, wherein a sample obtained from a patient is analyzed with regard to the presence of an MR1 ligand compound as identified herein.

This aspect is of particular use in selecting TCR molecules, or transgenic T cells or vectors for obtaining transgenic T cells from an autologous T cell population (“MR1-specific T cell reagents”), in a patient with a disease, particularly a tumour, that is characterized by a metabolic profile suggestive of certain of the MR1 ligands provided herein. It is even better suited to selecting MR1-specific T cell reagents for a patient whose tumour has been directly analysed and found to present certain of the MR1 ligand compounds disclosed herein. Selection of the best fitting/most specific MR1-specific T cell reagents will provide the best suited therapy for the patient, from a panel of MR1-specific T cell reagents available to the clinician.

Alternatively, the method can use an unbiased selection of T cells from which (a much smaller population) of T cells may be identified to increase the MR1-specific T cell reagent repertoire.

### Use of MR1 Ligand Compounds in Treatment or Prevention of MR1 Associated Diseases

The invention facilitates the use of T cells capable of specifically recognizing and reacting to an MHC-presented ligand that is presented by the invariant MR1 molecule, thereby providing a therapeutic option that is not restricted by the MHC genotype of the patient.

One aspect of the invention provides an MR1 ligand compound as specified above, or in any one of claims 1 to 9, for use in prophylaxis or treatment of a disease associated with an aberrant or absent MR1-specific T cell response, particularly in treatment of cancer characterized by tumor cells expressing MR1.

The inventors have first found evidence to prove that the compounds disclosed herein are specifically presented, and recognized, by T cells in the context of cancer. Hence, the MR1 ligand compounds disclosed herein lend themselves to use as “cancer vaccines” in the sense that their presence will enhance therapeutic approaches enabled by the MR1-T cell interaction.

In certain embodiments, the MR1 ligand compound is co-administered with an anticancer drug. As disclosed herein, the inventors have been able to demonstrate that the administration of paclitaxel or doxorubicin, established anti-tumour drugs, increase the presentation of MR1 ligand compounds as disclosed herein, presumably by increasing the metabolic stress inside cancer cells. Such combination is expected to provide a synergistic effect as it increases the load of MR1 ligands in the tissue.

In certain embodiments, the MR1 ligand compound is co-administered with a checkpoint modulator or checkpoint inhibitory agent. Such combination is expected to provide a synergistic effect as the downstream immune effect of MR1-ligand engagement with an MR1 specific T cell, physiologically present in the patient or possibly administered as part of an additional MR1 specific T cell therapy, is expected to be increased by removing inhibitory signals.

In particular embodiments, the compound is administered in association with (administered prior to, concomitant with or after administration of) a preparation comprising (transgenic) MR1-reactive T cells and/or a (transgenic) MR1-reactive T cell receptor polynucleotide construct (for example, a DNA expression construct or an RNA construct encoding an MR1-TCR or a viral vector having the same function).

Optionally, a polynucleotide expression vector encoding MR1 can be provided. Transgene expression of MR1 is a way to increase MR1 expression of tissue should affected disease tissue be found to down-regulate MR1 expression.

The MR1 ligand and/or the polynucleotide expression vector encoding MR1TCR or MR1 can be administered into a tumour, into the vicinity of the tumour, or into a lymph node draining the tumour site. It is expected that local increase of either agent is beneficial in comparison to systemic administration.

Specifically, the invention enables the administration of recombinant (allogeneic or autologous) T cells carrying a transgene TCR specifically capable of recognizing and reacting to MR1 presented disease specific MR1 ligand compounds.

The invention further enables the analysis of a T cell sample obtained from a patient for the presence of T cells capable of recognizing MR1 ligand compounds presented by MR1, to selectively stimulate and amplify, or to engineer de novo, such MR1 patient T cells for subsequent therapeutic administration.

The invention further enables the targeted administration of MR1 ligand compounds as specified herein to a patient to facilitate or amplify an MR1 specific T cell response or an MR1 targeted T cell therapy.

The compounds identified herein can be employed in methods pertaining to:
modulating an interaction between an MR1 (major histocompatibility complex class I-related gene protein 1) polypeptide and an MR1-specific T cell receptor molecule, wherein the MR1 polypeptide is contacted with the MR1 ligand compound;
generating a panel of MR1 multimeric reagents to use for identification and sorting of T cells reacting to MR1 ligand compounds presented on non-polymorphic MHC I-related MR1 antigen-presenting molecules;
identifying a TCR gene specifically reactive to said compounds to be used in a personalized cellular immunotherapy. This may be achieved by providing a preparation of tumor cells isolated from a patient, and submitting it to mass-spectrometry-based determination for assessing the presence and identity of the compounds.

In certain embodiments identifying a TCR gene specifically reactive to the compounds identified herein for use in a personalized cellular immunotherapy can be effected by the following general sequence of processes: 1) compound detection in a tumor biopsy (the tumor is classified based on the TAA presence) and then 2) guided TCR-mediated cellular immunotherapy based on the TCR best reacting to the identified TAA in complex with MR1.

Combination of MR1 Therapy with Pharmaceutical Drugs

An MR1 specific T cell response or an MR1 targeted T cell therapy may be further facilitated by combination with a pharmaceutical drug that increases the production of MR1 ligand compounds as specified herein.

The following pathways are targets of pharmaceutical drugs useful in this context: glutathione-S-transferase (GST), aldehyde reductases and aldehyde-keto reductases (AKR), aldehyde dehydrogenases (ALDH), aldehyde oxidase (AOX), xanthine oxidase (XO), Short chain reductase/oxidase (SDR).

The following compound classes and specific drugs identified as part of the compound class are possible combination partners to the MR1 TAAs in vaccination-plus-drug therapeutic approaches, or MR1-T-cell-transfer-plus-drug therapeutic approaches:

Glutathione-S-transferase (GST) inhibitors, particularly any one of the glutathione-S-transferase (GST) inhibitors selected from the group comprised of ethacrynic acid, Terrapin 199, Terrapin 286, Clofibrate, Gossypol, Indomethacin, Piriprost, Misonidazole, and Sulfasalazine.
Inhibitors of Aldo-Keto Reductase 1C3, particularly any one of the inhibitors of Aldo-Keto Reductase 1C3 selected from the group comprised of N-benzoylanthranilate, 2,3-arylpropenic acids, stylopne, 2′-hydroxyflavone, N-phenylsulfonyl indoles, N-(benzimidazolylcarbonyl)-piperidines, N-(indolylcarbonyl)-piperidines, N-(pyridinepyrroylylcarbonyl)-piperidines, N-(benzimidazoles or indole) benzoic acids, N-(phenylamino) benzoates, N-(naphthylamino) benzoates, Isoquinolones, Indomethacin analogs, Nitrogen or sulfur-substituted estrenes, β-Naphthylacetic acids, Baccharin, and Baccharin analogs.
Inhibitors of aldehyde dehydrogenases, particularly any one of the inhibitors of aldehyde dehydrogenases selected from the group comprised of Ampal, Benomyl, Citral, Chloral hydrate, Chlorpropamide analogs (NPI-1 and API-1), Coprine, Cyanamide, Daidzin, CVT-10216, 4-(Diethylamino)benzaldehyde (DEAB), Disulfirarn, Gossypol, Molinate, Nitroglycerin, Pargyline,
Inhibitors of aldehyde oxydase, particularly any one of the inhibitors of aldehyde oxydase selected from the group comprised of Amodiaquine, Chlorpromazine, Domperidone, Estradiol, Felopidine, Loratadine, Maprotiline, Metoclopramide, Norclomipramine, Nortriptyline, Ondansetron, Perphenazine, Promazine, Promethazine, Raloxifene, Salmeterol, Tacrine, Tamoxifen, and Thioridazine.
Xanthine oxidase inhibitors, particularly any one of the xanthine oxidase inhibitors selected from the group comprised of Allopurinol, Febuxostat, Oxypurinol, Tisopurine, topiroxostat, inositols (phytic acid and myo-inositol).
Glyoxalase I inhibitors, particularly any one of ethyl pyruvate, S-p-bromobenzyl glutathione cyclopentyl diester, S-ethylglutathione, 2-(8-chloro-2-(4-chlorophenyl)-3-hydroxy-4-oxochroman-6-yl)acetic acid.
GAPDH inhibitors, particularly any one of Gossypol, Koningic acid (heptelidic acid), arsenate, arsenic trioxide, 3-bromopyruvate, iodoacetate.

Hence, another aspect of the invention is the use of a compound which is capable of modulating the metabolism inside a cell, for example by inhibiting decarbonylation, in order to increase an MR1-dependent immune response. Exemplary drugs tested by the inventors include, but are not limited to, the group including paclitaxel, doxorubicin, disulfiram, daidzin, nitrobenzylthioinosine, ellagic acid, oleanoic acid, erythro-9-(2-hydroxy-3-nonyl) adenine (EHNA), pentostatin, 1-deazaadenosine and mycophenolic acid. These and related drugs can be employed for prophylaxis or therapy of a disease associated with aberrant or lacking MR1 expression, particularly treatment or prevention of recurrence of cancer disease associated with tumor cells expressing MR1.

Particularly useful examples of drugs providing an advantage according to this aspect of the invention, are pharmaceuticals capable of increasing the amount of reactive oxygen species (ROS), which leads to an excessive production of MR1 ligands inside the cell. These are: paclitaxel, docetaxel, epothilones, discodermolide, cabazitaxel, doxorubicin, daunorubicin, epirubicin, and idarubicin.

In one alternative of this aspect of the invention, paclitaxel is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Paclitaxel (CAS: 33069-62-4) is a taxane small molecule chemotherapeutic agent, with mitotic inhibitory properties. Paclitaxel interferes with the microtubule growth by binding to the @ subunit of tubulin. The resulting microtubule/paclitaxel complex does not have the ability to disassemble. Paclitaxel increases reactive oxygen species (ROS) levels significantly (Z. Yu, et al., ACS Nano 2015, 9, 11064; M. Li, et al., J. Am. Chem. Soc. 2018, 140, 14851; C. Dai et al., ACS Nano 2017, 11, 9467; P. Zhu, et al., ACS Nano 2018, 12, 3780; Jiang, H. et al., Small 2019, 15, 1901787). Paclitaxel is commonly administered as, but not limited thereto, a solution for intravenous injection with quantities up to 600 mg per treatment unit.

In one alternative of this aspect of the invention, docetaxel is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Docetaxel (CAS: 114977-28-5) is another taxane, clinically well-established and approved anti-mitotic chemotherapeutic agent, used mainly for treatment of patients suffering from breast, ovarian, and non-small cell lung cancer. Docetaxel binds reversibly to tubulin with high affinity in a 1:1 stoichiometric ratio. It promotes the assembly of microtubules from tubulin dimers and stabilizes microtubules by preventing depolymerization.

Despite the high antitumor efficiency of Docetaxel, its use is often limited due to the systemic and adverse side effects through excessive ROS production, Ca²⁺-influx, and inflammation markers such as tumor necrosis factor alpha (TNF-α), interleukin (IL)-1 beta (β), and IL-6 (Kütük, S. G et al., Biol Trace Elem Res 196, 184-194, 2020). Administrative forms include, solutions for intravenous and parenteral injection as well as powder for preparing such, with quantities up to 160 mg per treatment unit.

In one alternative of this aspect of the invention, epothilone is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Epothilones are a new class of compounds which can be isolated from a myxobacterium or semi- and/or fully synthetically synthesised and exhibit antimicrotubule effects. Epothilones share a similar mechanism of action to the taxanes but have demonstrated potent antiproliferative activity in several different multidrug-resistant tumors. Epothilones stabilize microtubules and induce apoptosis. Several Epothilones and derivatives/analogues thereof are currently in clinical trials. Members of this group include Epothilone A (CAS: 152044-53-6); Epothilone B (CAS: 152044-54-7), also known as Patupilone or BMS-310705 (a water-soluble derivative of Patupilone) and Ixabepilone (a second-generation semi-synthetic Patupilone; approved by the FDA in 2007) or Sagopilone (a fully synthetic third-generation analog of Patupilone), is a derivative of Epothilone A with a methyl group at the C12 atom. Recent studies have shown that Epothilone B causes mitochondrial collapse and release of ROS, thus promoting apoptosis. The above mentioned compounds are currently investigated in clinical trials, targeting anticancer and apoptosis promoting effects; Epothilone D (CAS: 189453-10-9) also known as KOS-862 or Desoxyepothilone B and KOS-1584 (a second-generation Epothilone D), is a small molecule currently investigated for treatment of colorectal cancer, lung cancer, breast cancer, solid tumors, and prostate cancer. Epothilone D lacks the C₁₂-13 epoxide and shows higher therapeutic potency than Epothilone A.

In one alternative of this aspect of the invention, discodermolide is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Discodermolide (CAS: 127943-53-7) is a small molecule with microtubule stabilizing properties similar to those of other taxols. Discodermolide induces ROS increase after treatment.

In one alternative of this aspect of the invention, cabazitaxel is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Cabazitaxel (CAS: 183133-96-2) is a small molecule, anti-neoplastic. Cabazitaxel is administered to people suffering from progressive prostate cancer despite treatment with docetaxel. Cabazitaxel modulates microtubule inhibition by binding to tubulin and promoting its assembly into microtubules while simultaneously inhibiting disassembly. This leads to the stabilization of microtubules, which results in the interference of mitotic and interphase cellular functions and therefore inhibition of further progression into the cell cycle, thus triggering apoptosis. Recent studies have shown that Cabazitaxel induces ROS production by inhibiting antioxidant-Sestrin 3 expression (Kosaka T. et al., Oncotarget. 2017; 8(50):87675-87683).

In one alternative of this aspect of the invention, doxorubicin is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Doxorubicin (CAS: 23214-92-8) is a well-studied and widely applicable, small molecule cytotoxic anthracycline antibiotic. Doxorubicin binds to nucleic acids, by specific intercalation of the planar anthracycline nucleus with the DNA double helix. Doxorubicin is provided to produce regression in disseminated neoplastic conditions such as, but not limited thereto, acute lymphoblastic leukemia, acute myeloblastic leukemia, Wilms' tumor, neuroblastoma, soft tissue and bone sarcomas, breast carcinoma, ovarian carcinoma, transitional cell bladder carcinoma, thyroid carcinoma, gastric carcinoma, Hodgkin's disease, malignant lymphoma and bronchogenic carcinoma. Doxorubicin is also indicated for use as a component of adjuvant therapy in women with evidence of axillary lymph node involvement following resection of primary breast cancer. Studies have shown that doxorubicin-induced ROS overproduction occurs inside mitochondria and is regulated by the mitochondrial NADPH oxidase (mitoNOX) activity (Asensio-López M C et al., PLoS One. 2017; 12(2):e0172803). Administrative forms of Doxorubicin include solutions for intravenous injection and powder for preparing such, with concentrations around 2 mg/mL.

In one alternative of this aspect of the invention, daunorubicin is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Daunorubicin (CAS: 20830-81-3) is a small molecule and toxic anthracycline aminoglycoside antineoplastic approved for treatment of patients suffering from leukemia and other neoplasms. Daunorubicin has antimitotic and cytotoxic activity through a number of proposed mechanisms of action, that are, inhibition of synthesis of macromolecules through intercalation of daunorubicin into DNA strands, interaction with molecular oxygen to produce ROS which further causes DNA damage via double strand breaks and topoisomerase II inhibition and the formation of DNA adducts (Al-Aamri H M. et al, BMC Cancer. 2019; 19(1):179).

In one alternative of this aspect of the invention, epirubicin is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Epirubicin (CAS: 56420-45-2) is a small molecule anthracycline which is the 4′-epi-isomer of doxorubicin and has antitumor effects due to its property to interfere with the synthesis and function of DNA. Epirubicin forms complexes with DNA by intercalation between base pairs, and it inhibits topoisomerase II activity by stabilizing the DNA-topoisomerase II complex, preventing the religation portion of the ligation-religation reaction that topoisomerase II catalyzes. Additionally, Epirubicin also interferes with DNA replication and transcription by inhibiting DNA helicase activity. Further studies showed that Epirubicin significantly increases ROS and mitochondrial H₂O₂ levels, which result in mitochondria-mediated apoptosis, triggered by the increased oxidative stress (Huang, T C. et al., Apoptosis 23, 226-236, 2018).

In one alternative of this aspect of the invention, idarubicin is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Idarubicin (CAS: 58957-92-9) is a small molecule anthracycline antineoplastic agent and is provided for patients suffering from, but not limited thereto, breast cancer, lymphomas and leukemias. (Celik H, Aring E. et al., J Pharm Pharm Sci. 2008; 11(4):68-82. PMID: 19183515).

Research showed that DNA damage by idarubicin occurs through a mechanism which involves its redox cycling with P450 reductase to generate ROS. DNA damage by idarubicin was found to increase with increasing concentrations of drug or enzyme as well as with increasing incubation time.

Disulfiram: In one alternative of this aspect of the invention, disulfiram is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1.

Disulfiram (CAS No. 97-77-8) is a well-characterized small molecule drug that has been used for more than 50 years for the treatment of alcoholism in humans. Studies have shown a degree of effectiveness against fungi (S. Khan et al., Jp. J. Med. Mycol (2007), 48, 109-113), protozoa (T. Nash and W. G. Rice, Antimicrob. Agents Chemother. (1998) 42, 1488-1492) and bacteria (MRSA, M. Phillips et al., Antimicrob. Agents Chemother. (1991), 35, 785-787) for disulfiram. The results shown in FIG. 7 support the compound's usefulness in administration as part of a MR1T targeted treatment (i.e. a prophylaxis or therapy of a disease associated with aberrant or lacking MR1 expression, particularly treatment or prevention of recurrence of cancer disease associated with tumor cells expressing MR1).

Mycophenolic acid: In another alternative of this aspect of the invention, mycophenolic acid is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Mycophenolic acid (CAS No. 24280-93-1) is a small molecule compound used as an immunosuppressant drug and anti-proliferative.

Daidzin: In another alternative of this aspect of the invention, daidzin is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1.

Daidzin is an isoflavone phytochemical (CAS No. 552-66-9). The results shown in FIG. 7 support the compound's usefulness in administration as part of a MR1T targeted treatment.

Yet another alternative of this aspect of the invention relates to nitrobenzylthioinosine (CAS No. 38048-32-7) for treatment of cancer disease associated with tumor cells expressing MR1, or prevention of recurrence of such tumor cells.

In yet another alternative of this aspect of the invention, ellagic acid (CAS No 476-66-4) and/or oleanoic acid (CAS No. 508-02-1) are provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. The results shown in FIG. 7 support the compounds' usefulness in administration as part of a MR1T targeted treatment.

In another alternative of this aspect of the invention, ethacrynic acid is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1.

Ethacrynic acid (CAS No. 58-54-8) is a commercial drug (commercial name: Edecrin) approved for treatment of high blood pressure and/or edema caused by diseases like congestive heart failure, liver failure and kidney failure. Ethacrynic acid was found to inhibit components of the Wnt/β-catenin pathway, inhibiting selective cytotoxicity towards CLL cells, as well as being a glutathione-S-transferase inhibitor. The small molecule contained in drugs is provided by various manufactures in different administrative forms such as tablets for oral intake and solutions for injections with dosages ranging from 25 mg (tablets) to 50 mg (in 50 mL solution for injection).

In another alternative of this aspect of the invention, Terrapin 199 is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Terrapin 199 (CAS No. 168682-53-9), also known as Ezatiostat is a small molecule drug with promising activity regarding treatment of patients suffering from myelodysplastic syndrome. Terrapin 199 is designed to inhibit a glutathione S-transferase and elevates activities of c-Jun NH₂ terminal kinase (JNK1) and ERK1/ERK2, allowing cells to proliferate under stress conditions which induces high levels of apoptosis. The drug is absorbed via oral intake (Hamilton et al. IDrugs. 2005 August; 8(8):662-9. PMID: 16044376).

In another alternative of this aspect of the invention, Terrapin 286 is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Terrapin 286 (CAS NO. 158382-37-7) also known as TLK-286 or Canfosamide is a small molecule, contained in drugs, and shows promising activity as an agent against various cancer forms (Rosen L S et al., Clin Cancer Res. 2004 Jun. 1; 10(11):3689-98. doi: 10.1158/1078-0432.CCR-03-0687. PMID: 15173075). TLK-286, when activated, splits into two fragments, one reacting with cell components such as RNA and DNA causing cell death, the other one being a glutathione analogue, thus blocking a glutathione S-transferase (Kavanagh J J et al., Int J Gynecol Cancer. 2005 Jul.-Aug. 15(4):593-600. doi: 10.1111/j.1525-1438.2005.00114.x. PMID: 16014111).

In another alternative of this aspect of the invention, clofibrate is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Clofibrate (CAS No. 637-07-0) is a fibric acid derivative used in commercially available drugs which are provided for treatment of hyperlipoproteinemia type III and hypertriglyceridemia. It inhibits glutathione S-transferase activity and additionally agonizes the PPAR-α receptor in soft tissues. This agonism ultimately leads to modification in gene expression resulting in increased beta-oxidation. The compound is provided as capsules (500-1000 mg) for oral intake.

In another alternative of this aspect of the invention, gossypol is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Gossypol (CAS No. 303-45-7) is a small molecule agent with glutathione-S-transferase inhibitory properties and a high potential regarding treatment of patients suffering from non-small cell lung cancer. Additionally, gossypol induces cell cycle arrest at the G0/G1 phase and inhibits cell-signalling enzymes, thus inhibiting the DNA replication resulting in apoptosis as well as inhibition of cell growth.

In another alternative of this aspect of the invention, indomethacin is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1.

Indomethacin (CAS No. 53-86-1) is a well-studied small molecule drug with anti-inflammatory properties. Indomethacin is a nonspecific and reversible inhibitor of both isoforms of the cyclo-oxygenase (COX) enzyme, or prostaglandin G/H synthase. Chemical bulk synthesis is conducted by several manufacturers, with the majority of commercially available products being administered orally, but are not limited thereto, with dosages raging from 25 mg to 75 mg per treatment.

In another alternative of this aspect of the invention, misonidazole is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Misonidazole (CAS No. 13551-87-6) is a small molecule compound in drugs with glutathione-S-transferase inhibitory properties and shows high potential regarding treatment of patients suffering from different tumor hypoxia. Misonidazole sensitizes hypoxic cells to the cytotoxic effects of ionizing radiation, resulting in inhibition of DNA synthesis.

In another alternative of this aspect of the invention, sulfasalazine is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Sulfasalazine (CAS No. 599-79-1) is a small molecule agent with glutathione-S-transferase inhibitory and anti-inflammatory properties. The drug is administered as tablet forms and rectal routes with dosages of ca. 500 mg per tablet.

In another alternative of this aspect of the invention, an N-benzoylanthranilate is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. N-benzoylanthranilates such as, but not limited thereto, commercially available meclofenamic acid comprise a group of compounds that inhibit AKR1C3 which is known to catalyze reactions stimulating tumor growth. The drugs are provided as tablets for oral intake but are not limited thereto. Dosages, particularly for meclofenamic acid range from 50 mg to 100 mg per treatment unit.

In another alternative of this aspect of the invention, an arylpropionic acid is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Arylpropionic acids, such as, but not limited thereto, commercially available ibuprofen, naproxen and flurbiprofen, comprise another group of compounds that exhibit inhibitory effect on AKR1C3. The drugs are administered mainly, but not limited thereto, by oral intake with dosages ranging from 50 mg to 1500 mg per treatment unit (Gazvoda M, et al., Eur J Med Chem. 2013 April; 62:89-97. doi: 10.1016/j.ejmech.2012.12.045. Epub 2013 Jan. 3. PMID: 23353746).

In another alternative of this aspect of the invention, a 2′-hydroxyflavone is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. 2′-Hydroxyflavone (2-(2-hydroxyphenyl)chromen-4-one; CAS NO. 35244-11-2) is a small molecule compound with AKR1C3 inhibitory properties and is associated with inhibiting tumor growth and/or reoccurrence of tumors.

In another alternative of this aspect of the invention, N-phenylsulfonyl indoles, N-(benzimidazolylcarbonyl)-piperidines, N-(indolylcarbonyl)-piperidines, N-(pyridinepyrroylylcarbonyl)-piperidines, N-(pyridinepyrroylylcarbonyl)-piperidines are provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. N-phenylsulfonyl indoles, N-(benzimidazolylcarbonyl)-piperidines, N-(indolylcarbonyl)-piperidines, N-(pyridinepyrroylylcarbonyl)-piperidines, N-(pyridinepyrroylylcarbonyl)-piperidines are compounds whose derivatives are nonsteroidal AKR1C3 inhibitors (Trevor M. Penning Expert Opin Ther Pat. 2017 Dec.; 27(12): 1329-1340. doi:10.1080/13543776.2017.1379503).

In another alternative of this aspect of the invention, β-naphthylacetic acid is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. β-naphthylacetic acids comprise a group of compounds that show selective inhibitory properties on AKR1C3 (Adeniji et al., J Med Chem. 2016; 59(16):7431-7444. doi:10.1021/acs.jmedchem.6b00160).

In another alternative of this aspect of the invention, baccharin is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Baccharin derivatives show high potential regarding inhibition of AKR1C3 (Kshitij V. et al., J. Med. Chem. 2019, 62, 3590-3616).

In another alternative of this aspect of the invention, AMPAL is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. 4-Amino-4-methyl-2-pentyne-1-al (AMPAL; CAS NO. 121188-32-7), is a small molecule compound known to inhibit aldehyde dehydrogenase activity and exhibit antitumor activity.

In another alternative of this aspect of the invention, Benomyl and carbendazim are provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Benomyl (N-[1-[(butylamino)carbonyl]-1H-benzimidazol-2-yl]-, methyl-ester; CAS NO. 17804-35-2) and its metabolite carbendazim (N-1H-benzimidazol-2-yl-, methyl ester; CAS NO. 10605-21-7) are small molecule compounds known to inhibit aldehyde dehydrogenase activity.

In another alternative of this aspect of the invention, Citral is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Citral (3,7-dimethyl-2,6-octadienal; CAS NO. 5392-40-5) is a naturally occurring compound found in herbs and citrus fruits with inhibitory properties regarding aldehyde dehydrogenases. Citral is already used in dietary supplement.

In another alternative of this aspect of the invention, chloral hydrate is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Chloral hydrate (2,2,2-trichloroethane-1,1-diol; CAS No. 302-17-0) is small molecule compound inhibiting an aldehyde dehydrogenase and is provided for treatment of insomnia. Various manufacturers are known; administrative forms include capsules and syrups for oral intake with dosages around 500 mg per capsule and 100 mg/mL.

In another alternative of this aspect of the invention, chlorpropamide is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Chlorpropamide (1-(4-chlorobenzenesulfonyl)-3-propylurea; 94-20-2) and analogues thereof are small molecule compounds known to irreversibly inhibit aldehyde dehydrogenase and are provided for treatment of non-insulin dependent diabetes mellitus.

In another alternative of this aspect of the invention, cyanamide is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Cyanamide (calcium (azanidylenemethylidene)azanide, CAS No. 420-04-2) in the form of citrated calcium salts are small molecules contained in drugs and commercially available and provided for treatment of alcoholism. Administrative forms are mainly, but not limiting thereto, tablets for oral intake with dosages around 50 mg per unit.

In another alternative of this aspect of the invention, CVT-10216 is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. CVT-10216 (3-[[[3-[4-[(methylsulfonyl)amino]phenyl]-4-oxo-4H-1-benzopyran-7yl]oxy]methyl]-benzoic acid; CAS NO. 1005334-57-5) is a small molecule and analogue of daidzin with aldehyde dehydrogenase inhibitory properties.

In another alternative of this aspect of the invention, DEAB is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. DEAB (4-(diethylamino)benzaldehyde; CAS NO. 120-21-8) is a commonly used selective inhibitor of aldehyde dehydrogenase in cancer stem cell biology.

In another alternative of this aspect of the invention, nitroglycerin is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1.

Nitroglycerin (1,3-bis(nitrooxy)propan-2-yl nitrate; CAS No. 55-63-0) is a small molecule with aldehyde dehydrogenase inhibitory properties and is provided for treatment of patients suffering from pain and high blood pressure amongst others. Nitroglycerin is a well characterized compound, which was first approved in 2000. Bulk chemical synthesis procedures are known; extensive pharmacological and toxicological data on the compound exists (Ignarro U et al., Proc Natl Acad Sci USA. 2002 Jun. 11; 99(12):7816-7. doi: 10.1073/pnas.132271799, PMID: 12060725; PMCID: PMC122975). Nitroglycerin is available in various administrative forms, including, but not limited thereto, spray forms, sublingual tablet forms, intravenous forms, extended-release tablet forms, transdermal forms, and capsules. The dosage is dependent of the administrative form and can vary from 200 μg to 160 mg.

In another alternative of this aspect of the invention, pargyline is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Pargyline (benzyl(methyl)(prop-2-yn-1-yl)amine; CAS No. 555-57-7) is a small molecule drug with aldehyde dehydrogenase inhibitory properties.

In another alternative of this aspect of the invention, amodiaquine is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1.

Amodiaquine (4-[(7-chloroquinolin-4-yl)amino]-2-[(diethylamino)methyl]phenol; CAS No. 86-42-0) is a small molecule with aldehyde oxydase inhibitory properties. Amodiaquine is a well characterized compound with synthesis known for more than 70 years. Amodiaquine is used as an antimalarial and anti-inflammatory drug and administered via oral intake with dosages around 100 mg per treatment unit.

In another alternative of this aspect of the invention, chlorpromazine, promazine and perphenazineare are provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Chlorpromazine ([3-(2-chloro-10H-phenothiazin-10-yl)propyl]dimethylamine; CAS NO. 50-53-3), Promazine (dimethyl[3-(10H-phenothiazin-10-yl)propyl]amine; CAS NO. 58-40-2) and Perphenazineare (2-{4-[3-(2-chloro-10H-phenothiazin-10-yl)propyl]piperazin-1-yl}ethan-1-ol; CAS No. 58-39-9) are small molecules contained in drugs which similarly exhibit aldehyde oxidase inhibitory properties and are provided for treatment of psychotic disorders. Chlorpromazine, Promazine and Perphenazineare are produced and marketed by a variety known manufacturers in different administrative forms such as, but not limited thereto, tablets for oral intake, solutions for intravenous, injection as well as syrups, with dosages ranging from 2 mg to 200 mg per treatment unit.

In another alternative of this aspect of the invention, domperidone is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1.

Domperidone (5-chloro-1-{1-[3-(2-oxo-2,3-dihydro-1H-1,3-benzodiazol-1-yl)propyl]piperidin-4-yl}-2,3-dihydro-1H-1,3-benzodiazol-2-one; CAS NO. 57808-66-9) is a small molecule contained in drugs which acts as a specific blocker of dopamine receptors and has aldehyde oxidase inhibitory properties. Domperidone synthesis is known to an expert (U.S. Pat. Nos. 4,066,772; 4,110,333; 4,126,687; 4,126,688; 4,160,836; and 4,175,129; Janssen Pharmaceutica NV). Administrative forms include tablets for oral intake with dosages around 10 mg per treatment unit.

In another alternative of this aspect of the invention, estradiol is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Estradiol (1S,3aS,3bR,9bS,11aS)-1-hydroxy-11a-methyl-1H,2H,3H,3aH,3bH,4H,5H,9bH, 10H,11H,11aH-cyclopenta[a]phenanthren-7-yl benzoate; CAS NO. 50-28-2) is a naturally occurring small molecule which is also contained in drugs which are provided in several hormone therapies for managing conditions with reduced estrogen. Additionally, Estradiol shows aldehyde oxidase inhibitory properties. A variety of administrative forms are provided: tablets for oral intake, sprays, gels and creams as well as injections and vaginal rings or transdermal patches.

In another alternative of this aspect of the invention, promethazine is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Promethazine (dimethyl[1-(10H-phenothiazin-10-yl)propan-2-yl]amine; CAS NO. 60-87-7) refers to a small molecule contained in drugs for treatment of a variety, but not limited thereto, of conditions such as acute allergic reactions, upper respiratory symptoms, anaphylaxis, pain, nausea and vomiting as well as exhibiting an inhibitory effect on aldehyde oxidases. The compound is well known to an expert, being approved almost 70 years ago by the FDA. Bulk chemical synthesis is well documented with various manufactures providing the compound in different administrative forms, such as tablets for oral intake, solutions for intravenous injection or suppositories for rectal administration with dosages ranging from 0.6 mg/mL to 50 mg per treatment unit.

In another alternative of this aspect of the invention, salmeterol is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1.

Salmeterol (CAS NO. 89365-50-4) is a small molecule which exhibits an inhibitory effect on aldehyde oxidases. The compound is contained in drugs which are provided for treatment of asthma and chronic obstructive pulmonary disease (COPD). The patient suffering from said conditions generally inhale the drug. Administrative forms include tablets with dosages of 20 μg to 50 μg per treatment unit.

In another alternative of this aspect of the invention, raloxifene is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1.

Raloxifene (CAS NO. 84449-90-1) is a well-documented small molecule with aldehyde oxidase inhibitory properties and mediates anti-estrogenic effects on breast cancer and uterine tissues as well as estrogenic effects on bone, i.e. preserving bone mineral density, and decrease the risk of breast.

In another alternative of this aspect of the invention, tacrine is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Tacrine (1,2,3,4-tetrahydroacridin-9-amine; CAS No. 321-64-2) is a small molecule drug provided as a respiratory stimulant and in treatment of Alzheimer's disease as well as other central nervous system disorders.

In another alternative of this aspect of the invention, Tamoxifen is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Tamoxifen (CAS NO. 10540-29-1) is a small molecule drug for treatment of various types of cancer disease such as, but not limited to, breast cancer, ovarian cancer and desmoid tumors. Tamoxifen is administered to patients via oral intake with dosages ranging from 10 mg to 40 mg per treatment unit.

In another alternative of this aspect of the invention, Allopurinol is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Allopurinol (CAS NO. 315-30-0) is a well characterized small molecule drug initially approved for treatment of gout. Allopurinol is a structural analog of hypoxanthine and after oral intake and subsequent ingestion metabolized to its active metabolite oxypurinol (alloxanthine) (CAS NO. 2465-59-0) which then acts as a xanthine oxidase inhibitor resulting in increased nucleotide concentration. Dosages can vary depending on the manufacturer providing the drug and range from 100 mg to 300 mg per treatment unit.

In another alternative of this aspect of the invention, Febuxostat is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Febuxostat (CAS NO. 144060-53-7) is a small molecule drug, provided for patients suffering from hyperuricemia. Febuxostat is a xanthine oxidase/dehydrogenase inhibitor decreasing serum uric acid after oral intake and ingestion by patients (Stamp L K et al., Intern Med J. 2007 April; 37(4):258-66). Administrative forms include tablets for oral intake with dosages ranging from 40 mg to 120 mg per treatment unit.

In another alternative of this aspect of the invention, tisopurine is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Tisopurine (CAS NO. 5334-23-6) is an alternative of Allopurinol.

In another alternative of this aspect of the invention, Topiroxostat is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Topiroxostat (CAS NO. 577778-58-6) developed for treatment of hyperuricemia and gout. The small molecule drug is approved in Japan since 2013 and marketed under the name Topiloric and Uriadec. The drug is absorbed via oral intake. Topiroxostat exerts its effect by competitive inhibition of xanthine oxidase.

In another alternative of this aspect of the invention, inositoles are provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Inositoles, particularly phytic acid (CAS No. 83-86-3) and myo-inositol (cyclohexane-1,2,3,4,5,6-hexol; CAS NO. 87-89-8) are small molecule contained in drugs and show high potential relating to treatment of various diseases such as depression, psychotic disorder and the prevention of cancer and cardiovascular calcifications as well as increasing fertility. A variety of drugs based on, or consisting of myo-inositols, indicated with the basic structure above, are already approved by the FDA. Dosages can vary depending on the manufacturer and typically range from 200 mg to 500 mg per treatment unit.

In another alternative of this aspect of the invention, ethyl pyruvate is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Ethyl pyruvate (ethyl 2-oxopropanoate; CAS NO. 617-35-6) is a small molecule and novel anti-inflammatory agent for treatment of patients suffering from critical inflammatory conditions. Ethyl pyruvate is an inhibitor of glyoxalase I and additionally inhibits the release of cytokines such as the TNF-alpha and HMGB1. Studies have shown promising anti-inflammatory and tissue protective activity (Kou, Q. Y. et al., Chinese critical care medicine 20(1), 34-36).

In another alternative of this aspect of the invention, S-p-bromobenzylglutathione cyclopentyl diester is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. S-p-bromobenzylglutathione cyclopentyl diester (N-[S-[(4-Bromophenyl)methyl]-N-L-gamma-glutamyl-L-cysteinyl]-Glycine dicyclopentyl ester; CAS NO. 166038-00-2) is a small molecule with inhibitory effect on glyoxalase I, furthermore inducing apoptosis (Thornalley P J et al., Biochem Pharmacol. 1996 May 17; 51(10):1365-72. doi: 10.1016/0006-2952(96)₀₀₀₅₉-7. PMID: 8787553.)

In another alternative of this aspect of the invention, arsenic trioxide is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. Arsenic trioxide (diarsorosooxidane; CAS NO. 1327-53-3), a small molecule chemotherapeutic agent is administered via intravenous injection, with concentrations of dosages raging from 1 mg/mL to 2 mg/mL, to patients suffering from cancer, particularly from APL. Arsenic trioxide causes morphological changes as well as fragmentation of the DNA leading to apoptosis in cancer cells by targeting and inhibiting the thioredoxin and glutathione system (Lu J. et al., Proc Natl Acad Sci USA. 2007 Jul. 24; 104(30):12288-93. doi: 10.1073/pnas.0701549104. Epub 2007 Jul. 18. PMID: 17640917; PMCID: PMC1940330).

In another alternative of this aspect of the invention, 3-bromopyruvate is provided for treatment of cancer disease associated with tumor cells expressing MR1, or prevention of recurrence of such tumor cells, particularly in combination with an MR1-reactive T cell, a MR1-reactive T cell receptor polynucleotide construct and/or a polynucleotide expression vector encoding MR1. 3-bromopyruvate (3-Bromo-2-oxopropanoic acid; CAS NO. 1113-59-3) is a selectively absorbed inhibitor of glycolysis in cancer cells and additionally triggers the release of apoptosis inducing factor (Cal M. et al. Cells. 2020; 9(5):1161. Published 2020 May 8. doi:10.3390/cells9051161).

Another aspect of the invention relates to the use of pharmaceutical drugs which facilitate the accumulation of nucleobases, thus increasing antigen availability. These drugs include, but are not limited to, the inhibitors of adenosine deaminase 1 (ADA1), erythro-9-(2-hydroxy-3-nonyl) adenine (EHNA), and inhibitors of both ADA1 and ADA2, pentostatin (CAS No. 53910-25-1) and 1-deazaadenosine. They also include inhibitors of dehydrogenase 1 (IMPDH1) such as, but not limited to, mycophenolic acid (CAS No. 24280-93-1). The results shown in FIG. 7 support the compounds' usefulness in administration as part of a MR1T targeted treatment.

A further aspect of the invention relates to the MIR1 ligand compounds identified herein according to the first aspect of the invention, particularly an MR1 ligand identified by any one of the formulas of claim 1 or their specific embodiments given in the dependent claims and Table 1, for use as a vaccine to elicit or boost an MR1T cell response, particularly in treatment or prevention of recurrence of cancer. In particular embodiments, the MR1 ligand compounds identified herein may be employed as a combination medicament to stimulate and augmenting antitumor activity of MR1T cells used in cellular therapy, and/or may be used in combination with immunostimulatory compounds and/or checkpoint modulator agents.

The MR1 ligand compounds identified herein may also be used as a vaccine in a subject without overt disease, but with a predisposition to develop such disease. Such as a human subject can be treated with preventative vaccination in advance of each of the maladies described herein.

Likewise, an alternative of this aspect of the invention relates to the MR1 ligand compounds identified herein according to the first aspect of the invention, particularly an MR1 ligand identified by any one of the formulas of claim 1 or their specific embodiments given in the dependent claims and Table 1, for use in a combination medicament in combination with oncotherapeutic agents.

One important advantage of the methods and compounds provided herein relates to the ability to test and provide TAAs to patients regardless of their MHC haplotype. TAA are the targets of clinically relevant anti-tumor immune response in cancer patients. Nevertheless, the majority of the so far identified TAAs are peptides presented by polymorphic MHC molecules. The extreme polymorphism of MHC genes limits the TAA targeting to those patients expressing certain MHC alleles. Targeting TAAs bound to MR1 non-polymorphic antigen-presenting molecules overcomes this constraint and is applicable to all patients bearing tumors expressing MR1. In addition, because tumor cells may express different non-peptidic TAAs, this strategy provides the possibility of targeting multiple antigens displayed by the same tumor cells, thus minimizing the potential occurrence of tumor escape-variants under selective immune pressure. Therefore, the identification of MR1-presented TAAs, matched with the specific MR1-restricted TCRs recognizing these antigens, has important implications for cancer immunotherapy.

In certain embodiments, the MR1 ligand compounds identified herein are provided for use in combination with an immune checkpoint modulator, particularly in combination with an immune checkpoint inhibitor agent.

In certain embodiments, the immune checkpoint inhibitor agent is ipilimumab (Yervoy; CAS No. 477202-00-9).

In certain embodiments, the immune checkpoint inhibitor agent is an inhibitor of interaction of programmed cell death protein 1 (PD-1) with its receptor PD-L1. In certain embodiments, the immune checkpoint inhibitor agent is selected from the clinically available antibody drugs nivolumab (Bristol-Myers Squibb; CAS No. 946414-94-4), pembrolizumab (Merck Inc.; CAS No. 1374853-91-4), pidilizumab (CAS No. 1036730-42-3), atezolizumab (Roche AG; CAS No. 1380723-44-3), and avelumab (Merck KGaA; CAS No. 1537032-82-8).

In certain embodiments, the MR1 ligand compounds identified herein are provided for use in combination, wherein any of the MR1 ligand compounds identified herein is a first combination partner, and
a) a modified T cell reactive to an MR1 molecule presenting the MR1 ligand compound and/or a nucleic acid expression vector encoding MR1 is a second combination partner, and
b) an immune checkpoint modulator, particularly an immune checkpoint inhibitor agent is a third combination partner.

Such combination is likely to be in a form where the combination partners are applied at different times and in different administration forms during treatment.

The identification of the compounds identified herein, which are presented by tumor cells in each tumor patient, represents a novel method to classify tumors. This classification is of relevance to select the appropriate MR1T-derived TCR to be used in a personalized TCR gene therapy.

Tumor patients can be also vaccinated with selected compounds previously detected in the tumor cells from the same patient. This treatment will have the goal of eliciting and/or stimulating MR1T cells specific for the compound and thus capable of recognizing and killing tumor cells.

Some of the MR1 ligand compounds described herein are also present in tissues of patients with autoimmune and metabolic diseases, including rheumatoid arthritis, systemic lupus erythematosus, type I diabetes, atherosclerosis, inflammatory bowel disease and multiple sclerosis. In general, disease with abnormal generation of Reactive Oxygen Species (ROS) are characterized by an accumulation of the MR1-binding compounds described below. In these cases, autoreactive MR1T cells that are stimulated during these diseases represent large T cell populations that may be inhibited in a therapeutic setting.

The inventors predict that appropriate therapeutic intervention forms can be found using those described for tumor immunotherapy as a template. The types of diseases which could be similarly treated because of the accumulation of the same types of MR1 ligands may thus be expanded.

Yet another aspect of the invention relates to the detection of MR1 ligand compounds identified herein as part of a method of disease classification, wherein the presence of at least one of the MR1 ligand compounds according to the invention is identified in samples from patients. In case of cancer patients, the identification of these compounds extracted from fresh tumor samples is considered a diagnostic marker. In certain embodiments, the TAA MR1 ligand compounds identified herein can be used for guiding cellular immunotherapy and also vaccination in combination with other therapeutic interventions, in other words, once the MR1 ligand is identified as present in the tumor, patients could be treated with personalized immunotherapy interventions including i) administration of pharmaceutical drugs capable of facilitating the accumulation of MR1T cell-stimulatory compounds as identified herein, ii) administration of selected TAA MR1 ligand compounds, iii) MR1 TCR-mediated cell therapy alone or in combination with other oncotherapeutic agents.

The invention also relates to a research method directed at the identification of T cells reactive to MR1-expressing cells. This encompasses a method to isolate from peripheral blood of normal donors or from patients suffering from cancer, metabolic or autoimmune diseases, MR1-restricted T cells sorted by using compound-loaded onto MR1 multimeric molecules. The cell source encompasses, but is not limited to, T cells isolated from tissue biopsies.

The invention also relates to the possibility to readily identify the TCR gene and protein sequences expressed by the above-mentioned T cells.

In certain aspects, the current invention is centered on the identification of novel classes of compounds which bind to non-polymorphic MR1 molecules. Some of the compounds identified herein modulate the surface expression of MR1. Some of the compounds identified herein (not necessarily the same compounds as those found to modulate the surface expression of MR1) are antigens, stimulating specific human T cells restricted to MR1. Some of the compounds identified herein were isolated from tumor cells, they were purified, identified and synthetic analogs were produced. The compounds showing antigenic activity, when presented in association with MR1 molecules, stimulate a population of human T cells discovered by the inventors and termed MR1T cells. Applications of this invention encompass, but are not limited to, the following methods.
i) a method to stimulate compound-specific T cells, to induce a prophylactic immune response,
ii) a method to stimulate compound-specific T cells, to induce a therapeutic immune response,
iii) a method for the identification and isolation of T cells reactive to the compounds,
iv) a method to modulate (increasing or decreasing) the quantity of defined compounds in the cells or presented by the cells,
v) a method to classify metabolically altered cells, including but not limited to tumor cells, according to the presence of the compounds.

Thus, in certain aspects and embodiments, the invention relates to the use of the MR1-associated compounds identified herein, for guiding personalized intervention of immunotherapy, of cellular immunotherapy, of vaccination strategies in people at risk and for diagnostics tests of several diseases, including cancer.

### Novel T Cell Receptors

Yet another aspect of the invention relates to novel isolated T cell receptors.

An isolated T cell receptor (TCR) according to the invention is constituted of an α chain of a T-cell receptor (TCR) and a β chain, or a γ and δ chain, of a TCR. The TCR specifically binds to and recognizes an MR1 ligand compound as specified herein, particularly in any one of claims 1 to 9, in association to an MR1 polypeptide,

The inventors have previously disclosed MR1 specific T cell receptor sequences (PCT/EP2019/074284, published as WO2020053312A1, incorporated herein by reference), which may be regarded as encompassed by the preceding definition to the extent that these previously published TCRs specifically recognize MR1-ligand complexes. Hence, the TCRs disclosed in WO2020053312A1, formed by association of SEQ ID NO 1 and 2, 3 and 4, 5 and 6, 13 and 25, 14 and 26, 15 and 27, 16 and 28, 17 and 29, 18 and 30, 19 and 31, 20 and 32, 21 and 33, 22 and 34, 23 and 35, 24 and 36 and 61 and 62 are disclaimed.

The inventors have found that a TCR as disclosed herein may be capable of binding to more than one MR1 ligand compound in the context of MR1 presentation. In this regard, the term “specifically binding” includes MR1 specific TCRs that bind to not only one but possibly a number of ligands disclosed herein.

The determination whether the TCR specifically binds to and recognizes an MR1 ligand compound can be made if upon interaction with MR1 and ligand compound, such TCR induces the activation of a T cell higher than the activation measured in the absence of said MR1 ligand. Such difference will have a statistical significance with a P value lower than or the same as 0.05 (P≤0.05). T cell activation can be evaluated with any of the following measurements: cytokine release, chemokine release, proliferation, expression of activation markers, target cell killing, induction of transcription factors or of reporter genes.

For the purpose of providing a definition of the TCR provided by definition of its binding to an MR1-MR1 ligand complex as claimed herein, the threshold for determining activation and hence, specificity of the TCR for an MR1 ligand complex, shall be determined as laid out in the methods section (activation assay) below, the determination being positive if a statistically significant positive effect is found for the MR1 ligand, particularly if the difference is at least 2-fold, more particularly if the difference is at least 10-fold.

In certain embodiments, an isolated TCR as provided herein recognizes the following compounds in association with MR1:
a. 1-methyladenosine (1);
b. 2-methyladenosine (2);
c. 2′-O-methyladenosine (3), with the proviso that the TCR composed of SEQ ID NO 22 and 34 is disclaimed;
d. N⁶, N⁶-dimethyladenosine (4), with the proviso that the TCRs composed of SEQ ID NO 1 and 2, and of 22 and 34 are disclaimed;
e. N⁶-threonylcarbamoyladenosine (5), with the proviso that the TCR composed of SEQ ID NO 22 and 34 is disclaimed;
f. N⁶-isopentenyladenosine (6), with the proviso that the TCRs composed of SEQ ID NO 1 and 2, 16 and 28, and of 22 and 34 are disclaimed;
g. N6-(cis-hydroxyisopentenyl) adenosine (7);
h. 2-methylthio-N⁶-(cis-hydroxyisopentenyl) adenosine (8), with the proviso that the TCRs composed of SEQ ID NO 1 and 2, 16 and 28, and of 22 and 34 are disclaimed;
i. 2-methylthio-N6-isopentenyladenosine (9);
j. N6-methyl-N⁶-threonylcarbamoyladenosine (10), with the proviso that the TCR composed of SEQ ID NO 22 and 34 is disclaimed;
k. 2′-O-ribosyladenosine (phosphate) (11);
l. N⁶-(3-Oxo-1-propenyl)-2′-deoxyadenosine (12), with the proviso that the TCRs composed of SEQ ID NO 1 and 2, 16 and 28, and of 22 and 34 are disclaimed;
m. 8-(9H-purin-6-yl)-2-oxa-8-azabicyclo[3.3.1]nona-3,6-diene-4,6-dicarbaldehyde (13), with the proviso that the TCR composed of SEQ ID NO 16 and 28 is disclaimed;
n. 1-(3-((2R,4S,5R)-4-hydroxy-5-(hydroxymethyl) tetrahydrofuran-2-yl)-3H-imidazo[2,1-i]purin-7-yl) heptan-2-one (14);
o. N2-methylguanosine (16);
p. 7-methylguanosine (17), with the proviso that the TCR composed of SEQ ID NO 22 and 34 is disclaimed;
q. 2′-O-methylguanosine (18);
r. N2, N2-dimethylguanosine (19), with the proviso that the TCR composed of SEQ ID NO 1 and 2 is disclaimed;
s. 2′-O-ribosylguanosine phosphate (20), with the proviso that the TCR composed of SEQ ID NO 22 and 34 is disclaimed;
t. 3-((2R,3R,4S,5R)-3,4-dihydroxy-5-(hydroxymethyl)tetrahydrofuran-2-yl)-6,7-dihydroxy-6-methyl-6,7-dihydro-3H-imidazo[1,2-a]purin-9(5H)-one (21), with the proviso that the TCR composed of SEQ ID NO 16 and 28 is disclaimed;
u. 3-((2R,3R,4S,5R)-3,4-dihydroxy-5-(hydroxymethyl)tetrahydrofuran-2-yl)-6,7-dihydroxy-7-methyl-6,7-dihydro-3H-imidazo[1,2-a]purin-9(5H)-one (22), with the proviso that the TCR composed of SEQ ID NO 16 and 28 is disclaimed;
v. 1-methylguanosine (24), with the proviso that the TCR composed of SEQ ID NO 22 and 34 is disclaimed;
w. 3-((2R,5R)-4-hydroxy-5-(hydroxymethyl) tetrahydrofuran-2-yl)-7-(2-oxoheptyl)-3H-imidazo[1,2-a]purin-9(5H)-one (25);
x. 2′-O-methylcytidine (29);
y. 3-methyluridine (30), with the proviso that the TCR composed of SEQ ID NO 22 and 34 is disclaimed;
z. 5-methyluridine (31);
aa. 3,2′-O-dimethyluridine (32);
bb. Queuosine (33);
cc. Wybutosine (34);
dd. Hydroxywybutosine (35);
ee. Pseudouridine (36);
ff. 6-((2R,4S,5R)-4-hydroxy-5-(hydroxymethyl)tetrahydrofuran-2-yl)-3-(2-oxoheptyl)-1,8a-dihydroimidazo [1,2-c]pyrimidin-5(6H)-one (37);
gg. N4-(3-oxo-1-propenyl)-2′-deoxycytidine (38), with the proviso that the TCR composed of SEQ ID NO 22 and 34 is disclaimed;
hh. 6-Methylmercaptopurine (40), with the proviso that the TCRs composed of SEQ ID NO 16 and 28, 22 and 34, and of 24 and 36 are disclaimed;
ii. N6-methyladenosine (42);
jj. 6-Methylpurine (43);
kk. 6-(Dimethylamino)purine (44);
ll. N6-(A2-Isopentenyl) adenine (45);
mm. 1-Methylguanine (47);
nn. N2-Methyl-2′-deoxyguanosine (48);
oo. 5′-Deoxy-5′-(methylthio)adenosine (52);
pp. N⁶-Methyl-2′-deoxyadenosine (53), with the proviso that the TCR composed of SEQ ID NO 3 and 4 is disclaimed;
qq. N⁶-(2-Hydroxyethyl)-2′-deoxyadenosine (54), with the proviso that the TCR composed of SEQ ID NO 3 and 4 is disclaimed.

In certain embodiments, the T cell receptor (TCR) provided by the invention recognizes the following compound in association with MR1:
a. N6-isopentenyladenosine (6), particularly wherein the TCR comprises the CDRs comprised of SEQ ID 99 and 100, particularly wherein the TCR comprises the polypeptide chains characterized by SEQ ID 97 and 98;
b. N⁶-(3-Oxo-1-propenyl)-2′-deoxyadenosine (12), particularly wherein the TCR comprises the CDRs comprised of SEQ ID 99 and 100, or 103 and 104, or 108 and 108, or 111 and 112, or 123 and 124, particularly wherein the TCR comprises the polypeptide chains characterized by SEQ ID 97 and 98, or 101 and 102, or 105 and 106, or 109 and 110, or 121 and 122;
c. 8-(9H-purin-6-yl)-2-oxa-8-azabicyclo[3.3.1]nona-3,6-diene-4,6-dicarbaldehyde (13), particularly wherein the TCR comprises the CDRs comprised of SEQ ID 99 and 100, or 103 and 104, or 107 and 108, or 111 and 112, or 123 and 124, particularly wherein the TCR comprises the polypeptide chains characterized by SEQ ID 97 and 98, or 101 and 102, or 105 and 106, or 109 and 110, or 121 and 122;
d. Pyrimido[1,2-a]purin-10(3H)-one (24), particularly wherein the TCR comprises the CDRs comprised of SEQ ID 99 and 100, or 103 and 104, or 111 and 112, particularly wherein the TCR comprises the polypeptide chains characterized by SEQ ID 97 and 98, or 101 and 102, or 109 and 110;
e. N⁴-(3-oxo-1-propenyl)-2′-deoxycytidine (38), particularly wherein the TCR comprises the CDRs comprised of SEQ ID 111 and 112, or 123 and 124, particularly wherein the TCR comprises the polypeptide chains characterized by SEQ ID 109 and 110, or 121 and 122;
f. 6-Methylmercaptopurine (40), particularly wherein the TCR comprises the CDRs comprised of SEQ ID 107 and 108, particularly wherein the TCR comprises the polypeptide chains characterized by SEQ ID 105 and 106.

The inventors have succeeded in providing novel TCRs specifically reactive to MR1 and MR1-MR1-ligand complexes. The invention provides an isolated T cell receptor (TCR) protein heterodimer comprising a TCR α chain and a TCR β chain, the TCR α chain and the TCR β chain each being characterized by a CDR3 sequence and the TCR protein heterodimer being characterized by a pair of α chain and β chain sequences selected from SEQ ID Nos 99 and 100, 103 and 104, 107 and 108, 111 and 112, 115 and 116, 119 and 120, 123 and 124, 127 and 128, 131 and 132.

In a particular embodiment thereof, the TCR α chain and the TCR β chain are selected from the pairs of α chain and β chain amino acid sequences of SEQ ID Nos 97 and 98, 101 and 102, 105 and 106, 109 and 110, 113 and 114, 117 and 118, 121 and 122, 125 and 126, and of 129 and 130 (see Table 3B), or a sequence at least 85% (>90%, 95%, 98%) identical to said pair of a chain and β chain amino acid sequences, and having the same biological activity as the original TCR.

The invention further provides a nucleotide sequence encoding the novel TCRs as described herein before. In particular embodiments, the polynucleotide is a DNA expression vector. In another particular embodiment, the polynucleotide encoding the TCR is an RNA molecule, particularly a stabilized messenger RNA molecule. In another particular embodiment, the polynucleotide encoding the TCR is a viral vector.

Another aspect of the invention relates to the isolated T cell expressing the TCR as defined by its binding properties above, or its sequence, or both, or the polynucleotide encoding same, for use in prophylaxis or treatment of a disease associated with an aberrant or absent MR1-specific T cell response, particularly for use in treatment of cancer.

A particular application of this aspect is its use in cancer characterized by MR1 expression.

In particular embodiments, the isolated T cell and/or the polynucleotide for use according to the invention are co-administered with an MR1 ligand compound as specified herein, particularly in any one of claims 1 to 9.

Alternatively, an MR1 ligand compound as specified herein, particularly in any one of claims 1 to 9, is provided for use in the treatment of cancer, co-administered in association with (administered prior to, concomitant with or after administration of) an isolated T cell expressing an MR1 specific TCR as specified in PCT/EP2019/074284, comprising a pair of α and β CDR3 sequences identified by the same line of Table 3, particularly an MR1 specific TCR constituted by SEQ ID NO 1 and 2, 3 and 4, 5 and 6, 13 and 25, 14 and 26, 15 and 27, 16 and 28, 17 and 29, 18 and 30, 19 and 31, 20 and 32, 21 and 33, 22 and 34, 23 and 35, 24 and 36 and 61 and 62 and/or a polynucleotide encoding said MR1 specific TCR, or a sequence at least 85% (>90%, 95%, 98%) identical to said pair of α chain and β chain amino acid sequences, and having the same biological activity as the original TCR.

The invention further provides an isolated T cell and/or the polynucleotide for use as specified in the preceding paragraph, wherein the isolated T cell expressing the TCR and/or the polynucleotide is co-administered with a pharmaceutical compound selected from paclitaxel, doxorubicin, docetaxel, cabazitaxel, daunorubicin, epirubicin, idarubicin, disulfiram, ellagic acid, pentostatin and mycophenolic acid (MPA) amodiaquine, chlorpromazine, domperidone, estradiol, felopidine, loratadine, maprotiline, metocloprarnide, nortriptyline, ondansetron, perphenazine, promazine, promethazine, raloxifene, salmeterol, tacrine, tamoxifen, and thioridazine, allopurinol, febuxostat, tisopurine, topiroxostat, inositols (phytic acid and myo-inositol).

In a particular embodiment, the isolated T cell expressing the TCR and/or the polynucleotide is co-administered with a pharmaceutical compound selected from paclitaxel, doxorubicin, disulfiram, and MPA, for treatment or prevention of a disease associated with aberrant or lacking MR1 expression, particularly treatment or prevention of recurrence of cancer disease associated with tumor cells expressing MR1.

In a more particular embodiment, the isolated T cell expressing the TCR and/or the polynucleotide is co-administered with a pharmaceutical compound selected from paclitaxel and doxorubicin, for treatment or prevention of a disease associated with aberrant or lacking MR1 expression, particularly treatment or prevention of recurrence of cancer disease associated with tumor cells expressing MR1.

The invention further provides the compound 8-(9H-purin-6-yl)-2-oxa-8-azabicyclo[3.3.1]nona-3,6-diene-4,6-dicarbaldehyde, first synthesized by the inventors and shown to be an MR1 ligand.

### Medical Treatment, Dosage Forms and Salts

Similarly, within the scope of the present invention is a method or treating a condition associated with a lack of MR1-specific T cell responses, or with too much of an MR1-specific T cell response, in a patient in need thereof, comprising administering to the patient a compound as specified in detail above.

Similarly, a dosage form for the prevention or treatment of a condition associated with a lack of MR1-specific T cell responses, or with too much of an MR1-specific T cell response is provided, comprising a non-agonist ligand or antisense molecule according to any of the above aspects or embodiments of the invention.

The skilled person is aware that any specifically mentioned drug may be present as a pharmaceutically acceptable salt of said drug. Pharmaceutically acceptable salts comprise the ionized drug and an oppositely charged counterion. Non-limiting examples of pharmaceutically acceptable anionic salt forms include acetate, benzoate, besylate, bitatrate, bromide, carbonate, chloride, citrate, edetate, edisylate, embonate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate, phosphate, diphosphate, salicylate, disalicylate, stearate, succinate, sulfate, tartrate, tosylate, triethiodide and valerate. Non-limiting examples of pharmaceutically acceptable cationic salt forms include aluminium, benzathine, calcium, ethylene diamine, lysine, magnesium, meglumine, potassium, procaine, sodium, tromethamine and zinc.

Dosage forms may be for enteral administration, such as nasal, buccal, rectal, transdermal or oral administration, or as an inhalation form or suppository. Alternatively, parenteral administration may be used, such as subcutaneous, intravenous, intrahepatic or intramuscular injection forms. Optionally, a pharmaceutically acceptable carrier and/or excipient may be present.

Topical administration is also within the scope of the advantageous uses of the invention. The skilled artisan is aware of a broad range of possible recipes for providing topical formulations, as exemplified by the content of Benson and Watkinson (Eds.), Topical and Transdermal Drug Delivery: Principles and Practice (1st Edition, Wiley 2011, ISBN-13: 978-0470450291); and Guy and Handcraft: Transdermal Drug Delivery Systems: Revised and Expanded (2^{nd} Ed., CRC Press 2002, ISBN-13: 978-0824708610); Osborne and Amann (Eds.): Topical Drug Delivery Formulations (1^{st} Ed. CRC Press 1989; ISBN-13: 978-0824781835).

### Pharmaceutical Compositions and Administration

Another aspect of the invention relates to a pharmaceutical composition comprising a compound as specified herein in the context of the present invention, or a pharmaceutically acceptable salt thereof, and a pharmaceutically acceptable carrier. In further embodiments, the composition comprises at least two pharmaceutically acceptable carriers, such as those described herein.

In certain embodiments of the invention, the compound of the present invention is typically formulated into pharmaceutical dosage forms to provide an easily controllable dosage of the drug and to give the patient an elegant product, which is easy to handle.

In embodiments of the invention relating to topical uses of the compounds of the invention, the pharmaceutical composition is formulated in a way that is suitable for topical administration such as aqueous solutions, suspensions, ointments, creams, gels or sprayable formulations, e.g., for delivery by aerosol or the like, comprising the active ingredient together with one or more of solubilizers, stabilizers, tonicity enhancing agents, buffers and preservatives that are known to those skilled in the art.

The pharmaceutical composition can be formulated for oral administration, parenteral administration, or rectal administration. In addition, the pharmaceutical compositions of the present invention can be made up in a solid form (including without limitation capsules, tablets, pills, granules, powders or suppositories), or in a liquid form (including without limitation solutions, suspensions or emulsions).

The dosage regimen for the compounds of the present invention will vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. In certain embodiments, the compounds of the invention may be administered in a single daily dose, or the total daily dosage may be administered in divided doses of two, three, or four times daily.

In certain embodiments, the pharmaceutical composition or combination of the present invention can be in unit dosage of about 1-1000 mg of active ingredient(s) for a subject of about 50-70 kg. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The pharmaceutical compositions of the present invention can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc. They may be produced by standard processes, for instance by conventional mixing, granulating, dissolving or lyophilizing processes. Many such procedures and methods for preparing pharmaceutical compositions are known in the art, see for example L. Lachman et al. The Theory and Practice of Industrial Pharmacy, 4th Ed, 2013 (ISBN 8123922892).

“Swiss-type” method of manufacture: An alternative aspect of the invention relates to the use of an MR1 ligand compound as described in table 1 in the manufacture of a medicament for prophylaxis or treatment of a disease associated with an aberrant or absent MR1-specific T cell response, particularly in treatment of cancer characterized by tumor cells expressing MR1, optionally in combination with a drug recited in claim 27. Another embodiment relates to the use of an isolated T cell receptor as described in claim 16 to 20 in the manufacture of a medicament for prophylaxis or treatment of a disease associated with an aberrant or absent MR1-specific T cell response, particularly in treatment of cancer characterized by tumor cells expressing MR1, optionally in combination with a drug recited in claim 27. Another embodiment relates to the use of an isolated T cell as described in claim 22 in the manufacture of a medicament for prophylaxis or treatment of a disease associated with an aberrant or absent MR1-specific T cell response, particularly in treatment of cancer characterized by tumor cells expressing MR1, optionally in combination with a drug recited in claim 27. Another embodiment relates to the use of a polynucleotide encoding a TCR as described in claim 21 in the manufacture of a medicament for prophylaxis or treatment of a disease associated with an aberrant or absent MR1-specific T cell response, particularly in treatment of cancer characterized by tumor cells expressing MR1, optionally in combination with a drug recited in claim 27.

Wherever alternatives for single separable features are laid out herein as “embodiments”, it is to be understood that such alternatives may be combined freely to form discrete embodiments of the invention disclosed herein.

The invention is further illustrated by the following examples and figures, from which further embodiments and advantages can be drawn. These examples are meant to illustrate the invention but not to limit its scope.

### DESCRIPTION OF THE FIGURES

FIG. 1 shows an example of cell surface MR1 upregulation on APCs by two compounds. a) 2-Methyladenosine and b) N6,N6-dimethyladenosine were used at the three indicated concentrations. Expression of MR1 on THP1-MR1 cells was assessed by flow cytometry and is shown as median fluorescence intensity (MFI) compared to the baseline expression on the cells incubated with vehicle only.

FIG. 2 shows an example of a competition assay for stimulation of three MR1T cell clones. 2-Methyladenosine was used with THP-1 cells at the 3 indicated concentrations, before adding the optimal dose of antigen for each of the illustrated T cell clones (DGB129, MCA2E7 and TC5A87). The response of the T cell clones is shown as mean±sd of IFN-γ release. The T cell response in the presence of antigen alone, of compound alone (circle) or of THP-1 cells only (triangle) are also shown.

FIG. 3 shows an example of compound-specific MR1T cell activation. The response of three MR1T cell clones DGB129 (a), MCA2E7 (b) and TC5A87 (c) to THP-1 cells incubated with the compound N6, N6-dimethyladenosine (M6,2A) is shown as mean±sd of the IFN-γ released after overnight stimulation. The response of the T cells to THP-1 cells and compound vehicle are shown as control.

FIG. 4 shows the generation of M₃ADE-loaded MR1 monomers: a) Gel filtration chromatography purification of MR1 protein refolded in the presence of M₃ADE. Absorption at 280 nm and retention time (min) are shown on the y- and x-axis, respectively. The indicated peaks 1, 2, and 3 were collected and b) were used to activate DGB129 cells in a plate-bound assay. IL-13 released by the T cells is shown as mean±sd of duplicates.

FIG. 5 shows the validation of MR1-M₃ADE tetramer staining. The MR1T cell clone AVA34 was generated by two rounds of FACS sorting of CD3⁺, MR1-M₃ADE tetramer⁺ cells from PBMCs, followed by PHA stimulation and cloning by limiting dilution of the M₃ADE-reactive T cells. a) Histogram overlay showing the staining results of AVA34 cells with MR1-M₃ADE, and MR1-5-OP-RU tetramers as well as with MR1-M₃ADE tetramer after incubation with anti-TCR mAb specific for Vβ8 (JR2, 1 μg/ml), which prevented M₃ADE tetramer staining.
b) Bar chart showing IL-13 release by AVA34 cells toward THP-1 cells in the presence of M₃ADE. Low levels of IL-13 are released in the presence of MGdA, but not of the other indicated compounds. A375-MR1 cells were used as positive control and T cells alone as negative control.

FIG. 6 shows the ex vivo frequency of MR1-M₃ADE tetramer⁺ MR1T cells in the blood of healthy donors. Dot plots of PBMCs from 9 healthy donors co-stained with MR1-M₃ADE tetramer and anti-CD3 (UCHT1). Each plot represents an individual donor. Cells were gated as live CD3⁺, CD14⁻, CD19⁻ single cells and numbers indicate the percentage of MR1-M₃ADE tetramer⁺ MR1T cells within the oval gate.

FIG. 7 shows the treatment of APCs with drugs that induce accumulation of carbonyl-containing molecules stimulate MR1T cells. a) Daidzin and b) Disulfiram increase the response of MCA2B9 MR1T clone. c) Disulfiram induces the stimulation of the MCA2B1 MR1T clone when exogenous deoxycytidine is co-administered. d) Oleanoic acid induces the stimulation of the TC5A87 MR1T clone, e) Ellagic acid induces the stimulation of the QY1A16 MR1T clone, f) EHNA induces the stimulation of the TC5A87 MR1T clone, and g) Mycophenolic acid induces the stimulation of the TC5A87 MR1T clone. APCs (THP-1 cells) were incubated 18 h with individual drugs before addition of MR1T cells and of deoxycytidine (only in panel c). Drugs did not induce stimulation of MR1T cells in the absence of APCs. The responses of the T cell clones are expressed as IFN-γ release (mean±sd) of triplicates.

FIG. 8 shows purine metabolism involved in MR1T antigen accumulation. (A-F) MR1T clones TC5A87 (A-C) and DGB129 (D-F) reactivity against A375-MR1 cells transduced with sgRNAs targeting ADA (▴, A and D), LACC1 (▾, B and E), ADSSL1 (▪, C and F) or scrambled sgRNA control (O, A-F). (G) Activation of MR1T clones TC5A87 (left), DGB129 (middle) and MCA3C3 (right) by THP-1 cells pre-incubated with 250 μM of the indicated molecules or A375-MR1 or vehicle. IFN-γ released is presented as mean±SD of triplicate cultures. The experiments shown are representative of at least three independently performed ones. One representative experiment of at least three independent replicates is shown in each panel. *p<0.05, **p≤0.01 and ***p≤0.001 compared to matching control (A-F, Multiple t-test) or compared to vehicle (G, One-way Anova with Dunnett's multiple comparison).

FIG. 9 shows glycolysis and methylglyoxal lead to MR1T antigen accumulation. Schematic representation of methylglyoxal generation. Dihydroxyacetone phosphate (DHAP), glyceraldehyde 3-phosphate (G3P). (A and B) Stimulation of MR1T cell clone TC5A87 (A) and DGB129 (B) with A375-MR1 cells transduced with sgRNAs targeting TPI1 (●) or scrambled control (◯). (C and D) Stimulation of MR1T cell clone TC5A87 (C) and DGB129 (D) in response to fixed A375-MR1 cells incubated for 6 h with different concentrations of D-(+) Glucose (◯) or 2-deoxy-D-Glucose (▪) before fixation. (E and F) Stimulation of MR1T cell clone TC5A87 (E) and DGB129 (F) with A375-MR1 cells transduced with sgRNAs against GLO1 (▪), scrambled sgRNAs control (◯) or a vector to overexpress GLO1 (▾). (G and H) Stimulation of MR1T cell clone TC5A87 (G) and DGB129 (H) with THP-1 cells pre-treated with 25 μM erythro-9-(2-hydroxy-3-nonyl) adenine hydrochloride (EHNA), 10 μM mycophenolic acid (MPA) and 20 μM S-bromobenzylglutathione (BBG), alone or in combination. (I-L) MR1T clone DGB129 activation in response to THP-1 cells (◯), GLO1-overexpressing (▾) and GLO1 ko (▪) THP-1 cells, in the presence of Methylglyoxal (1, J) or deoxyadenosine (K, L). IFN-γ released is presented as mean±SD of triplicate cultures. The data shown are representative of at least three independent experiments. (A, L) Mean±SD, n=3, *p<0.05 **p<0.01 and ***p<0.001. (A, B and I-L) Multiple t-test, (C and D) one-way Anova with Dunnett's multiple comparison, (E and F) two-way Anova with Dunnett's multiple comparison, (G and H) one-way Anova with Tukey's multiple comparison.

FIG. 10 shows aldehyde contributing and scavenging MR1T antigen accumulation (A-C) Stimulation of MR1T cell clone TC5A87 (A), DGB129 (B) and MCA3C3 (C) with THP-1 cells pre-treated with Doxorubicin (75 nM) or Paclitaxel (5 μM) in the absence or presence of nucleosides (dAdenosine and Guanosine, both 150 μM). (D-F) Stimulation of MR1T cell clone TC5A87 (D), MCA2B1 (E) MCA3C3 (F) with fixed A375-MR1 cells treated with buthionine sulfoximine (400 μM, BSO), glutathione (4 mM, GSH), N-acetylcysteine (4 mM, NAC) and apocynin (100 μM, APO). (G-1) Stimulation of MR1T cell clone TC5A87 (G), DGB129 (H) MCA2B1 (1) with fixed A375-MR1 cells treated with ML-210 (6 μM), RSL-3 (1 μM) and mercaptosuccinic acid (3.3 μM, MSA). (J-L) Stimulation of MR1T cell clone TC5A87 (J), DGB129 (K) and MCA2B1 (L) with fixed A375-MR1 cells treated with hydralazine (100 μM) or aminoguanidine (5 mM). IFN-γ release is presented as the mean±SD of triplicate cultures. The data shown are representative of at least three independent experiments. (A-L) Mean±SD, n=3, *p<0.05, **p<0.01 and ***p<0.001. (A-C) two-way Anova with Tukey's multiple comparison, (D-L) one-way Anova with Dunnett's multiple comparison. See also FIGS. 15 and 16.

FIG. 11 shows synthetic MDA or MG nucleoside adducts induce MR1 upregulation and stimulate MR1T T cells. (A-E) Five synthetic adducts M₃ADE (A), OPdA (B), M1G (C), MGG (D) and OPdC (E) were purified and their structures are indicated on the left. (Middle) Upregulation of MR1 surface expression on THP-1 MR1 cells following 6 hours incubation with the adducts indicated in each panel. MFI fold change±SD of the staining with anti-MR1 mAbs is graphed. (Right) IFN-γ release response of several MR1T cell clones co-cultured overnight with THP-1 cells in the presence (◯) or absence of adducts (●). Blocking of T cell reactivity by anti-MR1 mAbs is also shown for the highest antigen (Ag) dose (▴). Data are shown as mean of triplicate cultures±SD. Experiments were performed at least three independent times, with one representative experiment shown.

FIG. 12 shows MR1T cell clones recognize unmodified nucleoside adducts and different tumour cells. (A) Recognition of plate-bound soluble MR1 (▪) loaded (◯) with M₃ADE, OPdA, M₁G and OPdC by MR1T clones AVA34, QY1A16, AC1A4 and TC5A87, respectively. (B) Activation assay of 14 MR1T cell clones in the presence of THP-1 cells treated with each of the indicated antigens or vehicle. Stimulation of 14 MR1T cell clones in the presence of THP-1 cells treated with M₃ADE (from 100 μM), OPdA (100 μM), M1G (from 300 μM) or OPdC (100 μM) or vehicle. Heat-map reports the cube root of mean IFN-γ concentration. (C) MR1T clones recognize different tumour cells. Black bars depict co-cultures with indicated tumour cell line in the presence of anti-HLA-A,B,C mAbs (clone W6/32), anti-HLA-DR mAbs (clone L243), white bars represent co-cultures with both anti-HLA and anti-MR1 mAbs. Graph shows IFN-γ release (mean±SD of triplicate cultures). Data are representative of mean of triplicate cultures±SD (A, C) and of at least two independent experiments (A-C). (C) **p<0.01 and ***p<0.001 (Multiple t-test) compared to anti-MR1 blocking control.

FIG. 13 shows M₃ADE specific cells are detected in the periphery of healthy donors. (A) Histograms of MR1-M₃ADE tetramer staining of the MR1T cell clone AC1A4 compared to the MAIT cell clone MRC25. (B) Histograms of AVA34 cell staining (a representative clone derived from MR1-M₃ADE tetramer-sorting) using MR1 tetramers loaded with 5-OP-RU (light grey), 6-FP (dark grey) and M₃ADE (black). (C) Activation of clone AVA34 with 5 synthetic DNA adducts, including relevant blocking with anti-MR1 mAbs. Columns show IFN-γ release (mean±SD of triplicate cultures). (D) MR1-M₃ADE tetramer staining of PBMCs from 2 representative donors plotted against CD3 expression. Cells are pre-gated on live, single cells and frequency is displayed as a percentage of CD3⁺ T cells. (E) Summary of the ex vivo frequency of MR1-M₃ADE tetramer⁺ T cells from the peripheral blood of 9 healthy donors. Horizontal bar represents median value. (F) Dot plots display the proportion of CD4⁺/CD8⁻, CD4⁻/CD8⁺ and CD4⁻/CD8⁻ (DN) T cells within MR1-M₃ADE tetramer⁺ T cells. Horizontal bars represent median values. (G) Dot plots display the percentages of Naïve, Central memory, Effector memory and terminally differentiated effector memory (T_{EMRA}) T cells within the MR1-M₃ADE tetramer⁺ T cell fraction. Horizontal bars represent median values. (H) Histograms of surface expression of CD8, CD4, CD45RA and CCR7 markers on T cells pre-gated as M₃ADE-MR1 tetramer⁺, CD3⁺ from each donor (D1-D9). T cell clone staining results are representative of at least two independent experiments.

FIG. 14 shows M₃ADE tetramer⁺ T cells are present in human squamous cell lung carcinoma. (A and B) Dot plots show MR1-M₃ADE tetramer⁺, CD3⁺ T cells within TILs from two patients (Donor 840 and 895) after in vitro expansion. The percentage of MR1-M₃ADE tetramer⁺, CD3⁺ T cells within total CD3⁺ cells is displayed (Left). Dot plots show the proportion of CD4⁺/CD8⁻, CD4⁻/CD8⁺ and CD4⁻/CD8⁻ T cells within MR1-M₃ADE tetramer⁺ T cells. Numbers in each quadrant indicate the percentage of each population calculated on total M₃ADE-MR1 tetramer⁺, CD3⁺ T cells (Right). (C) Dot plots show the FACS-sorted M₃ADE-tetramer enriched T-cell lines derived from TILs of each donor activated in the presence of A375-MR1-B2M KO cells, or the same cells over-expressing MR1, treated with M₃ADE or treated with M₃ADE and blocked with anti-MR1 mAbs. T-cell activation was measured by TCR downregulation, shown as both the percentage and MFI of tetramer⁺ cells indicated in each plot. (D) Activation of TIL lines in each of the conditions corresponding to C. IFN-γ release (mean±SD) was measured in duplicate for each condition. The data shown are representative of at least two independent experiments. (E) MR1-dependent activation of tetramer-positive T cells by measuring IFN-γ release.

FIG. 15 shows characterization of knock-out cell lines. (A, D, G, J, M) Activation assay of MAIT clone MRC25 in response to A375-MR1 cells and 5-OP-RU. (P and S) Activation assay of MRC25 with THP-1 cells and 5-OP-RU. Cells are either wild type (◯), knock-out (A ▴, D ▪, G ▾, J ◯, M ▾, S ▾) or overexpressing (M ▪, P ▪) the indicated genes. IFN-γ is expressed as mean±SD of triplicate independent cultures. (B, E, H, K, N, Q, T) Surface MR1 expression of the genetic engineered cell lines. MR1 staining of wild type cells (dark grey shadow), ko lines (black line) and GLO1-overexpressing (GLO1⁺⁺) A375-MR1 (N, grey thick clashed line) with anti-MR1 mAbs 26.5. Isotype-matched control staining is depicted in wild type cells (light grey shadow with grey dot line), in ko cells (black dashed line) or GLO1⁺⁺ A375-MR1 (N, black dotted line). (C, F, I, L, O, R, U) Western blot analysis of target protein expression in indicated cell lines. Tubulin or Actin were used as loading control. The experiments were repeated at least twice and one representative experiment is shown.

FIG. 16 shows stimulation of MAIT clone MRC25 with nucleobases, inhibitory drugs and synthetic antigens. (A) MAIT clone MRC25 was stimulated with THP-1 cells in the presence of different nucleobases (250 μM), Methylglyoxal (250 μM) or 5-OP-RU (30 nM). (B and D) MRC25 cells were stimulated with THP-1 cells treated with indicated drugs. (C) MRC25 cells were stimulated with A375-MR1 cells treated with GSH, NAC, APO, BSO or GPX inhibitors and fixed or with THP-1 cells pulsed with 5-OP-RU (10 nM). (E) MRC25 cells were stimulated with A375-MR1 cells treated with carbonyl scavengers at indicated concentrations and fixed before T cell addition (empty bars). As controls, the same experiment was performed with the same carbonyl scavengers in the presence of 6,7-dimethyl-8-ribityllumazine (20 μM, black bars). (F) MRC25 cells were stimulated with THP-1 cells in the presence of M₃ADE, OPdA, OPdC (all 100 μM), M₁G (13 μM) or 5-OP-RU (10 nM). n.d.=not determined ***p<0.001 compared to vehicle-treated cells using One-way ANOVA (A, B, C, F) or Two-way ANOVA (D and E) with Dunnett's multiple comparison. IFN-γ is expressed as mean±SD of triplicate independent cultures. The experiments were repeated at least twice and one representative experiment is shown.

FIG. 17 (A) Quantification of ROS produced in THP-1 cells treated with Doxorubicin, Paclitaxel or Phorbol 12-myristate 13-acetate (PMA). Results are expressed Median Fluorescence Intensity (MFI) of live cells±SD of triplicate independent cultures. (B) Surface expression in indicated tumor cell lines of MR1 (black line) or HLA A,B,C (grey dotted line). Isotype matching staining control is depicted as grey shade. (C) Table reporting tissue origin and diagnosed disease of each cell line. Each experiment was repeated at least twice and one representative experiment is shown. **p 0.01 and ***p 0.001 using one-way Anova with Dunnett's multiple comparison.

FIG. 18 shows cell surface MR1 upregulation on APCs by selected compounds. Expression of MR1 on tumor cells was assessed by flow cytometry and is shown as median fluorescence intensity (MFI) compared to the baseline expression on the cells incubated with vehicle only.

FIG. 19 shows compound-induced MR1T cell activation. The responses of MR1T cell clones to THP-1 cells incubated with different doses of each compound or with the fixed dose of 50 μM (black bars) are shown as mean±sd of the IFN-γ released after overnight stimulation. The response of the T cells to THP-1 cells and compound vehicle (white bars) are shown as control.

FIG. 20 shows the capacity of different compounds to compete for stimulation of MR1T cells. Each compound was used with THP-1 cells at the 3 indicated concentrations, before adding the optimal dose of antigen for specific MR1T cells. The T cell response is shown as mean±sd of IFN-γ release. The T cell responses in the presence of competitor and antigen (square), of compound alone (triangle) or of THP-1 cells only (circle) are shown.

TABLE 1
List of exemplary active compounds with structures and references.

| | | |
|---|---|---|
| Full name Short name CAS Number Molecular Formula Molecular Weight Ref. | 1-methyladenosine (1) m1A 15763-06-1 C₁₁H₁₅N₅O₄ 281.27 Ishiwata, Itoh et al. 1995 Seidel, Brunner et al. 2006 Cayman Chemical, Cat. 16937 | 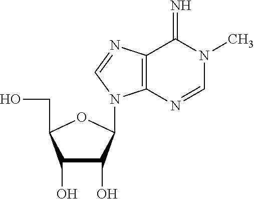 |
| Full name Short name CAS Number Molecular Formula Molecular Weight | 2-methyladenosine (2) m2A 16526-56-0 C₁₁H₁₅N₅O₄ 281.27 Santa Cruz, sc-500888 | 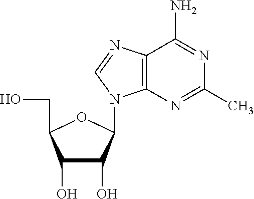 |
| Full name Short name CAS Number Molecular Formula Molecular Weight | 2′-O-methyladenosine (3) Am 2140-79-6 C₁₁H₁₅N₅O₄ 281.27 Cayman Chemical, 16936 | 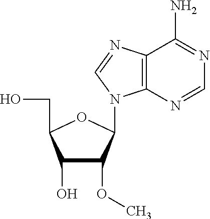 |
| Full name Short name CAS Number Molecular Formula Molecular Weight | N6,N6-dimethyladenosine (4) m6,6A or m6,2A 2620-62-4 C₁₂H₁₇N₅O₄ 295.29 | 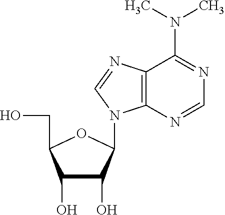 |
| Full name Short name CAS Number Molecular Formula Molecular Weight | N6-threonylcarbamoyladenosine (5) t6A 24719-82-2 C₁₅H₂₀N₆O₈ 412.35 Toronto Research Chemicals, T405560 | 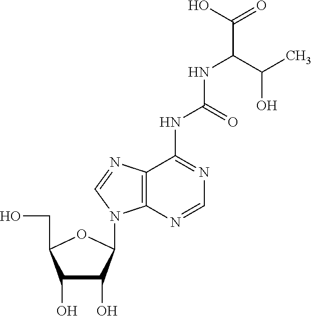 |
| Full name Short name CAS Number Molecular Formula Molecular Weight | N6-isopent-2-enyladenosine (6) i6A 7724-76-7 C₁₅H₂₁N₅O₄ 335.36 Toronto Research Chemicals, I821840 | 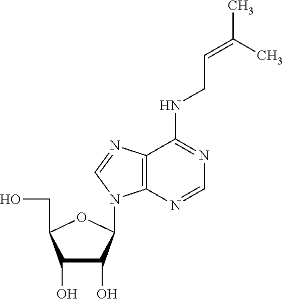 |
| Full name Short name CAS Number Molecular Formula Molecular Weight Ref. | N6-(cis-hydroxyisopent-2-enyl) adenosine (7) io6A 15896-46-5 C₁₅H₂₁N₅O₅ 351.36 NA | 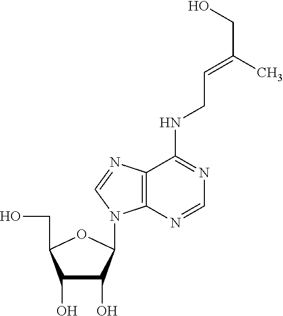 |
| Full name Short name CAS Number Molecular Formula Molecular Weight | 2-methylthio-N6-(cis-hydroxyisopent-2-enyl) adenosine (8) ms2io6A 52049-48-6 C₁₆H₂₃N₅O₅S 397.45 Toronto Research Chemicals, M330525 | 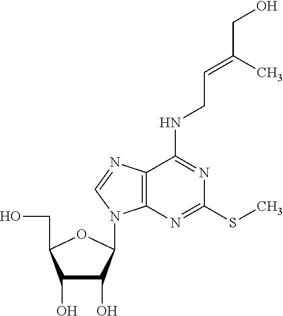 |
| Full name Short name CAS Number Molecular Formula Molecular Weight | 2-methylthio-N6-isopent-2-enyladenosine (9) ms2i6A 20859-00-1 C₁₆H₂₃N₅O₄S 381.45 Toronto Research Chemicals, M330675 | 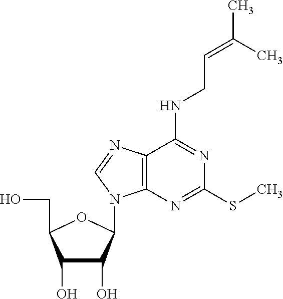 |
| Full name Short name CAS Number Molecular Formula Molecular Weight | N6-methyl-N6-threonylcarbamoyladenosine (10) m6t6A 39667-81-7 C₁₆H₂₂N₆O₈ 426.38 | 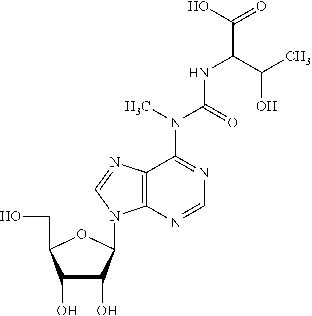 |
| Full name Short name CAS Number Molecular Formula Molecular Weight | 2′-O-ribosyladenosine (phosphate) (11) Ar(p) 28050-13-7 C₁₅H₂₂N₅O₁₁P₁ 479.34 | 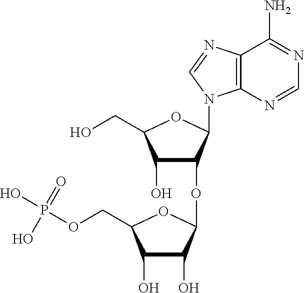 |
| Full name Short name CAS Number Molecular Formula Molecular Weight Ref. | N6-(3-oxo-1-propenyl)-2′-deoxyadenosine (synthetic adduct) (12) M₁dA or OPdA 178427-43-5 C₁₃H₁₅N₅O₄ 305.11 (confirmed by MS) Voulgaridou, Anestopoulos et al. 2011 Marnett 2002 Stone et al. 1990 | 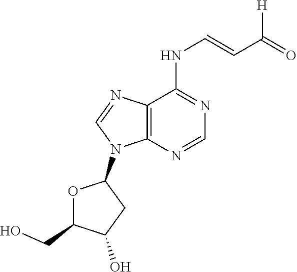 [SMILES: O=C/C=C/Nc1ncnc2c1ncn2[C@H]1C[C@H](O)[C@@H](CO)O1] |
| Full name Short name CAS Number Molecular Formula Molecular Weight | 8-(9H-purin-6-yl)-2-oxa-8-azabicyclo[3.3.1]nona- 3,6-diene-4,6-dicarbaldehyde (13) M₃ADE (novel compd, no CAS No) C₁₄H₁₁N₅O₃ 297.09 Confirmed by MS and NMR | 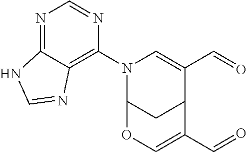 [SMILES: O=CC1=COC2CC1C(C=O)=CN2c1ncnc2[nH]cnc12] |
| Full name Short name CAS Number Molecular Formula Molecular Weight Ref. | 1-(3-((2R,4S,5R)-4-hydroxy-5-(hydroxymethyl) tetrahydrofuran-2-yl)-3H-imidazo[2,1-i] purin-7-yl)heptan-2-one (synthetic adduct) (14) Heptanone-1, N²-ε-dA or ONEdA 289709-63-3 C₁₉H₂₅N₅O₄ 389.21 Geacintov and Broyde 2010 Voulgaridou, Anestopoulos et al. 2011 Kawai and Nuka 2018 Company, Cat. NA | 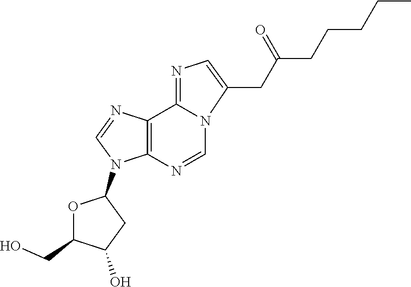 [SMILES: CCCCCC(=O)Cc1cnc2c3ncn([C@H]4C[C@H](O)[C@@H](CO)O4)c3ncn12] Proposed structure |
| Full name Short name CAS Number Molecular Formula Molecular Weight Ref. | 1-methylguanosine (15) me1G 2140-65-0 C₁₁H₁₅N₅O₅ 297.27 Seidel, Brunner et al. 2006, ibid. Toronto Research Chemicals, B426593 | 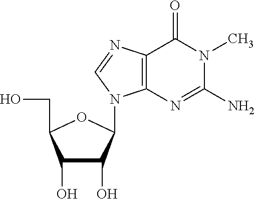 |
| Full name Short name CAS Number Molecular Formula Molecular Weight Ref. | N2-methylguanosine (16) m2G 2140-77-4 C₁₁H₁₅N₅O₅ 297.27 Seidel, Brunner et al. 2006, ibid. Santa Cruz, sc-215517 | 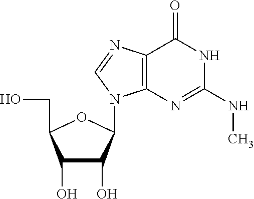 |
| Full name Short name CAS Number Molecular Formula Molecular Weight | 7-methylguanosine (17) m7G 20244-86-4 C₁₁H₁₇N₅O₅ 299.27 Sigma-Aldrich, M0627 | 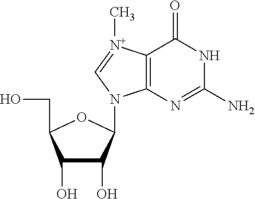 |
| Full name Short name CAS Number Molecular Formula Molecular Weight | 2′-O-methylguanosine (18) Gm 2140-71-8 C₁₁H₁₅N₅O₅ 297.27 Cayman, 21039 | 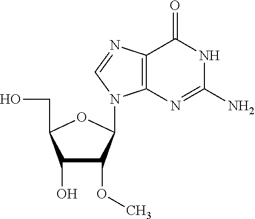 |
| Full name Short name CAS Number Molecular Formula Molecular Weight Ref. | N2,N2-dimethylguanosine (19) m²₂G 2140-67-2 C₁₂H₁₇N₅O₅ 311.30 Seidel, Brunner et al. 2006 | 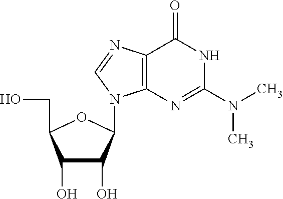 |
| Full name Short name CAS Number Molecular Formula Molecular Weight Ref. | 2′-O-ribosylguanosine (20) Gr 148711-49-3 C₁₅H₂N₅O₉ 415.13 Mikhailov et al, Journal of Carbohydrate Chemistry (1997), 16(1), 75-92 Company, Cat. NA | 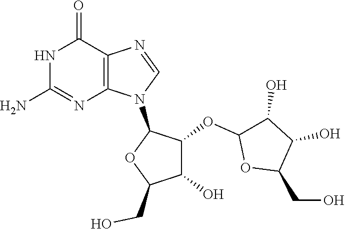 [SMILES: Nc1nc2c(ncn2[C@@H]2O[C@H](CO)[C@@H](O)[C@H]2OC2O[C@H](CO)[C@@H](O)[C@H]2O)c(=O)[nH]1] |
| Full name Short name: CAS Number Molecular Formula Molecular Weight Ref. | 3-((2R,3R,4S,5R)-3,4-dihydroxy-5- (hydroxymethyl)tetrahydrofuran-2-yl)- 6,7-dihydroxy-6-methyl-6,7-dihydro-3H- imidazo[1,2-a]purin-9(5H)-one (21) MGG (6M) 159062-85-8 C₁₃H₁₇N₅O₆ 355.11 Rabbani et al., Biochemical Society Transactions (2014), 42(2), 425-432 | MGG (6M) 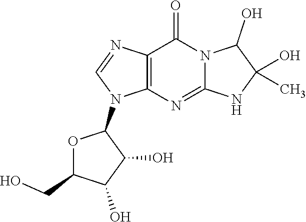 [SMILES: CC1(O)Nc2nc3c(ncn3[C@@H]3O[C@H](CO)[C@@H](O)[C@H]3O)c(=O)n2C1O] |
| Full name Short name: CAS Number Molecular Formula Molecular Weight Ref. | 3-((2R,3R,4S,5R)-3,4-dihydroxy-5- (hydroxymethyl)tetrahydrofuran-2-yl)- 6,7-dihydroxy-7-methyl-6,7-dihydro-3H- imidazo[1,2-a]purin-9(5H)-one (22) MGG (7M) 1254179-20-8 C₁₃H₁₇N₅O₆ 355.11 Rabbani et al., ibid. | MGG (7M) 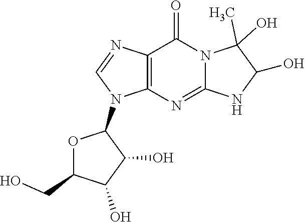 [SMILES: CC1(O)C(O)Nc2nc3c(ncn3[C@@H]3O[C@H](CO)[C@@H](O)[C@H]3O)c(=O)n21] |
| Full name Short name CAS Number Molecular Formula Molecular Weight Ref. | 2-((6-oxo-6,7-dihydro-1H-purin-2-yl)amino) propanoate (23) CEdG or 2-(1-Carbohyethyl)guanine 13262-60-7 C₁₃H₁₆N₅O₇ 354.11 Petrova, Millsap et al., Chemical Research in Toxicology 2014, 27(6), 1019-1029 | 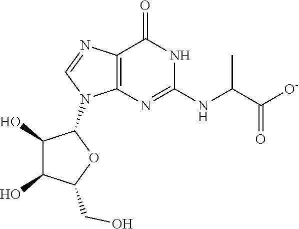 [SMILES: CC(Nc1nc2c(ncn2[C@@H]2O[C@H](CO)[C@@H](O)[C@H]2O)c(=O)[nH]1)C(=O)[O-]] |
| Full name Short name CAS Number Molecular Formula Molecular Weight | pyrimido(1,2-a)purin-10(1H)-one (24) M1G 103408-45-3 C8H5N5O 187.161 Hadley and Draper 1990 Marnett 1999; Yates, Dempster et al. 2017 Toronto Research Chemicals, P997400 | 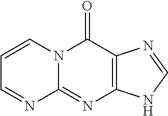 [SMILES: O=c1c2nc[nH]c2nc2ncccn12] |
| Full name Short name CAS Number Molecular Formula Molecular Weight Ref. | 3-((2R,5R)-4-hydroxy-5-(hydroxymethyl) tetrahydrofuran-2-yl)-7-(2-oxoheptyl)-3H- imidazo[1,2-a]purin-9(5H)-one (synthetic adduct) (25) Heptanone-1,N²-ε-dG or ONEdG 423718-43-8 C₁₉H₂₅N₅O₅ 403.19 Geacintov and Broyde 2010 Voulgaridou, Anestopoulos et al. 2011 Kawai and Nuka 2018 | 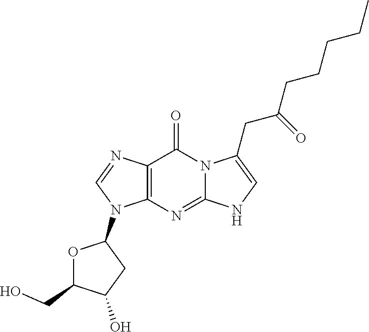 [SMILES: CCCCCC(=O)Cc1c[nH]c2nc3c(ncn3[C@H]3C[C@H](O)[C@@H](CO)O3)c(=O)n12] |
| Full name Short name CAS Number Molecular Formula Molecular Weight Ref. | 3-(2-deoxy-β-D-erythro-pentofuranosyl)pyrimido [1,2-α]purin-10(3H)-one (synthetic adduct) (26) M₁dG 87171-83-3 C₁₃H₁₃N₅O₄ 303.27 Voulgaridou, Anestopoulos et al. 2011 Marnett 2002 Wauchope, Beavers et al. 2015 Riggins, Daniels et al. 2004 Stone et al. 1990 | 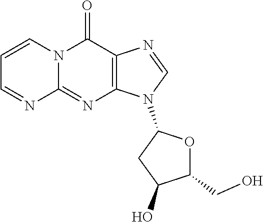 [SMILES: O=c1c2ncn([C@H]3C[C@H](O)[C@@H](CO)O3)c2nc2ncccn12] |
| Full name Short name CAS Number Molecular Formula Molecular Weight Ref. | N2-oxopropenyl-deoxyguanosine (synthetic adduct) (27) N²OPdG 189241-16-5 C₁₃H₁₄N₅O₅ 303.28 Voulgaridou, Anestopoulos et al. 2011 Marnett 2002 Wauchope, Beavers et al. 2015 Riggins, Daniels et al. 2004 | 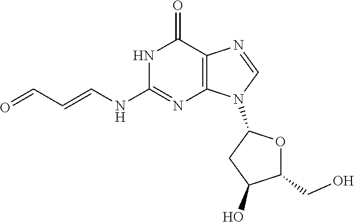 [SMILES: O=C/C=C/Nc1nc2c(ncn2[C@H]2C[C@H](O)[C@@H](CO)O2)c(=O)[nH]1] |
| Full name Short name CAS Number Molecular Formula Molecular Weight Ref. | 3-((2R,4S,5R)-4-hydroxy-5- (hydroxymethyl)tetrahydrofuran-2-yl)-12-oxo- 5,6,10,12-tetra hydro-3H-6,10- methano[1,3,5]oxadiazocino[5,4-a]purine-9- carbaldehyde (28) M2dG 945684-11-7, 189103-82-0, 189103-83-1 C₁₆H₁₇N₅O₆ 375.12 Marnettet al., JAGS (1986), 108(6), 1348-50 | 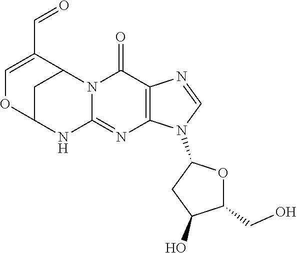 [SMILES: O=CC1=COC2CC1n1c(nc3c(ncn3[C@H]3C[C@H](O)[C@@H](CO)O3)c1=O)N2] |
| Full name Short name CAS Number Molecular Formula Molecular Weight Ref. | 2′-O-methylcytidine (29) Cm 2140-72-9 C₁₀H₁₅N₃O₅ 257.25 NA TCI Chemicals, M2317 | 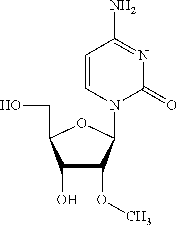 |
| Full name Short name CAS Number Molecular Formula Molecular Weight Ref. | 3-methyluridine (30) m3U 2140-69-4 C₁₀H₁₄N₂O₆ 258.23 NA | 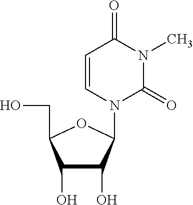 |
| Full name Short name CAS number Molecular Formula Molecular Weight | 5-methyluridine (31) m5U 1463-10-1 C₁₀H₁₄N₂O₆ 258.23 Sigma-Aldrich, 535893 | 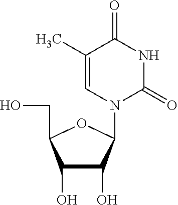 |
| Full name Short name CAS number Molecular Formula Molecular Weight Ref. | 3,2′-O-dimethyluridine (32) m3Um NA C₁₁H₁₆N₂O₆ 272.26 NA | 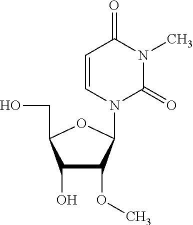 |
| Full name Short name CAS number Molecular Formula Molecular Weight | Queuosine (33) Q 57072-36-3 C₁₇H₂₃N₅O₇ 409.39 Toronto Research Chemicals, N925205 | 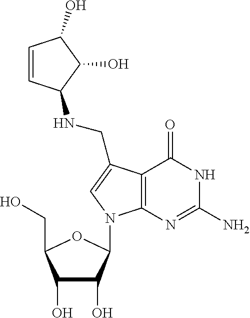 |
| Full name Short name CAS number Molecular Formula Molecular Weight | Wybutosine (34) yW 55196-46-8 C₂₁H₂₈N₆O₉ 508.49 | 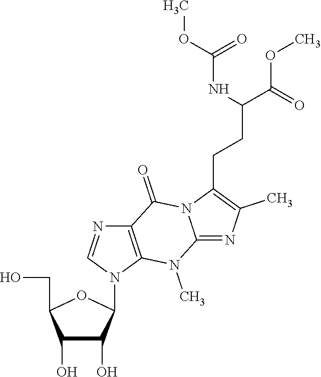 |
| Full name Short name CAS number Molecular Formula Molecular Weight Ref. | Hydroxywybutosine (35) OHyW NA C₂₁H₂₈N₆O₁₀ 524.49 Lu, Zhiwei et al Journal of Chromatography B: Analytical Technologies in the Biomedical and Life Sciences (2017), 1051, 108-117 | 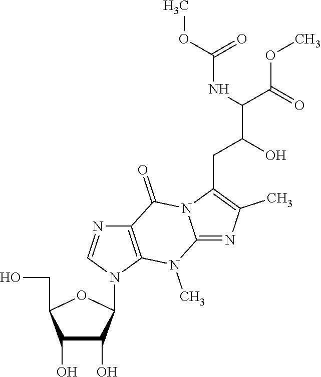 |
| Full name Short name CAS number Molecular Formula Molecular Weight | 5-(3,4-dihydroxy-5-(hydroxymethyl)tetrahydrofuran- 2-yl)pyrimidine-2,4(1H,3H)-dione (36) Pseudouridine, Psi 1445-07-4 C₉H₁₂N₂O₆ 244.20 | 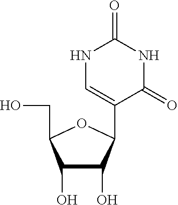 |
| Full name Short name CAS number Molecular Formula Molecular Weight Ref. | 6-((2R,4S,5R)-4-hydroxy-5- (hydroxymethyl)tetrahydrofuran-2-yl)-3-(2- oxoheptyl)-1,8a-dihydroimidazo[1,2-c]pyrimidin- 5(6H)-one (synthetic adduct) (37) Heptanone-1,N²-ε-dC or ONEdC 566943-69-9 C₁₈H₂₅N₃O₅ 365.20 Voulgaridou, Anestopoulos et al. 2011 Kawai and Nuka 2018 | 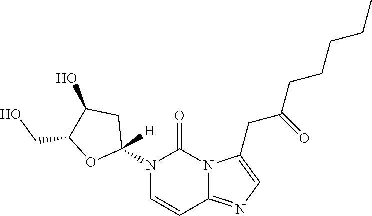 [SMILES: CCCCCC(=O)Cc1cnc2ccn([C@H]3C[C@H](O)[C@@H](CO)O3)c(=O)n12] |
| Full name Short name CAS number Molecular Formula Molecular Weight Ref. | N4-(3-oxo-1-propenyl)-2′-deoxycytidine (synthetic adduct) (38) M₁dC or OPdC 666718-05-4 C₁₂H₁₅N₃O₅ 281.10 Voulgaridou, Anestopoulos et al. 2011 Marnett 2002 Szekely et al. Nucleosides, Nucleotides & Nucleic Acids (2008), 27(2), 103-109 | 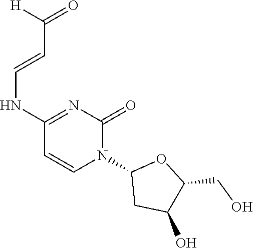 [SMILES: O=C/C=C/Nc1ccn([C@H]2C[C@H](O)[C@@H](CO)O2)c(=O)n1] |
| Full name Short name CAS number Molecular Formula Molecular Weight | 8-(1-((2R,4S,5R)-4-hydroxy-5- (hydroxymethyl)tetrahydrofuran-2-yl)-2-oxo-1,2- dihydropyrimidin-4-yl)-2-oxa-8-azabicyclo[3.3.1] nona-3,6-diene-4,6 dicarbaldehyde (39) M3dC 129104-26-3 C₁₈H₁₉N₃O₇ 389.12 | 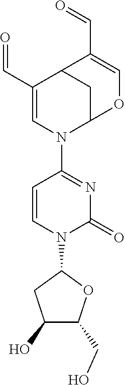 [SMILES: O=CC1=COC2CC1C(C=O)=CN2c1ccn([C@H]2C[C@H](O)[C@@H](CO)O2)c(=O)n1] |
| Full name Short name CAS number Molecular Formula Molecular Weight | (2R,3S,4R,5R)-2-(hydroxymethyl)-5-(6- (methylthio)-9H-purin-9-yl)tetrahydrofuran-3,4- diol (40) 6-MMPr 342-69-8 C₁₁H₁₄N₄O₄S 298.32 Sigma-Aldrich, M4002 | 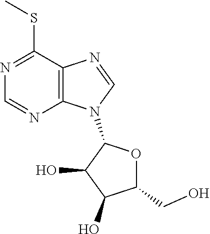 [SMILES: CSc1ncnc2c1ncn2[C@@H]1O[C@H](CO)[C@@H](O)[C@H]1O] |
| Full name Short name CAS Number Molecular Formula Molecular Weight | 3-Methyladenine (41) 3MA 5142-23-4 C₆H₇N₅ 149.15 Cayman CAY-13242 | 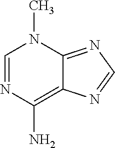 |
| Full name Short name CAS Number Molecular Formula Molecular Weight | N6-methyladenosine (42) m6A 1867-73-8 C₁₁O₄N₅H₁₅ 281 Toronto Research Chemicals, M275895 | 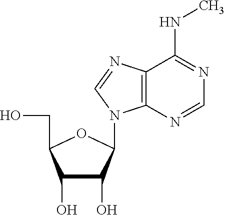 |
| Full name Short name CAS Number Molecular Formula Molecular Weight | 6-Methylpurine (43) MeP 2004-03-7 C₆H₆N₄ 134.14 Sigma-Aldrich, M6502 | 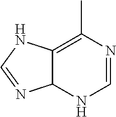 |
| Full name Short name CAS Number Molecular Formula Molecular Weight | 6-(Dimethylamino)purine (44) 6DMAP 938-55-6 C₇H₉N₅ 163.18 Sigma-Aldrich, D2629 | 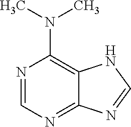 |
| Full name Short name CAS Number Molecular Formula Molecular Weight | N6-(Δ2-Isopent-2-enyl) adenine (45) i6Ade (also noted as tautomer 2iP; 6-(γ,γ- Dimethylallylamino)purine) 2365-40-4 C₁₀H₁₃N₅ 203.2 Cayman, CAY-17906; Sigma: D7660 | 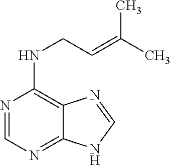 |
| Full name Short name CAS Number Molecular Formula Molecular Weight | N1-Methyl-2′-deoxyguanosine (46) N1MedG 5132-79-6 C₁₁H₁₅N₅O₄ 281.27 Toronto Research Chemicals, M293030 | 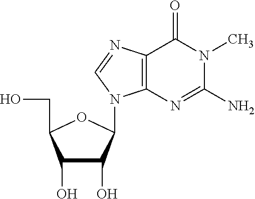 |
| Full name Short name CAS Number Molecular Formula Molecular Weight | 1-Methylguanine (47) MeG 938-85-2 C₆H₇N₅O 165.15 Sigma-Aldrich, 67070 | 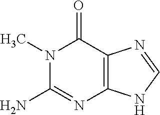 |
| Full name Short name CAS Number Molecular Formula Molecular Weight | N2-Methyl-2′-deoxyguanosine (48) N2MedG 19916-77-9 C₁₁H₁₅N₅O₄ 281.27 Toronto Research Chemicals, M293035 | 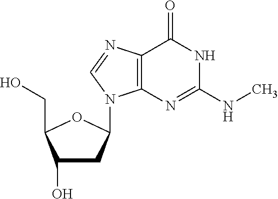 |
| Full name Short name CAS Number Molecular Formula Molecular Weight | 7-Methyl-7-deaza-2′-deoxyguanosine (49) 7Medeaza2dG 90358-21-7 C₁₂H₁₆N₄O₄ 280.28 Toronto Research Chemicals, M299590 | 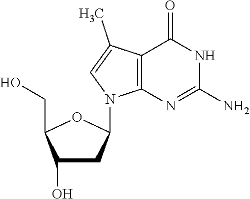 |
| Full name Short name CAS Number Molecular Formula Molecular Weight | O6-Methyl-2′-deoxyguanosine (50) O⁶MedG 964-21-6 C₁₁H₁₅N₅O₄ 281.27 Toronto Research Chemicals, M293040 | 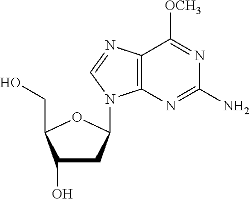 |
| Full name Short name CAS Number Molecular Formula Molecular Weight | N2-Ethyl-2′-deoxyguanosine (51) N2EtdG 101803-03-6 C₁₂H₁₇N₅O₄ 295.29 Sigma-Aldrich, N3289 | 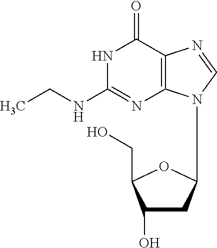 |
| Full name Short name CAS Number Molecular Formula Molecular Weight | 5′-Deoxy-5′-(methylthio)adenosine (52) MTA 2457-80-9 C₁₁H₁₅N₅O₃S 297.33 Sigma-Aldrich, D5011 | 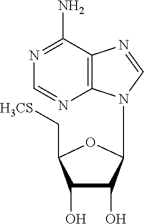 |
| Full name Short name CAS Number Molecular Formula Molecular Weight | N6-Methyl-2′-deoxyadenosine (53) N⁶MedAdo 2002-35-9 C₁₁H₁₅N₅O₃ 265.27 Sigma-Aldrich, M2389 | 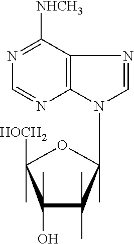 |
| Full name Short name CAS Number Molecular Formula Molecular Weight | N6-(2-Hydroxyethyl)-2′-deoxyadenosine (54) N6HEdA 137058-94-7 C₁₂H₁₇N₅O₄ 295.29 Toronto Research Chemicals, H941985 | 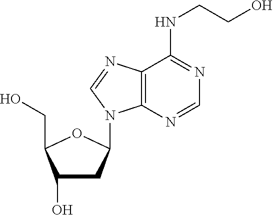 |
| Full name Short name CAS Number Molecular Formula Molecular Weight | O6-(2-Hydroxyethyl)-2′-deoxyguanosine (55) O6HEdG 111447-35-9 C₁₂H₁₇N₅O₅ 311.29 Toronto Research Chemicals, H942020 | 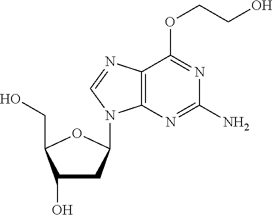 |
| Full name Short name CAS Number Molecular Formula Molecular Weight | N6-Succinyl Adenosine (56) SAdo 4542-23-8 C₁₄H₁₇N₅O₈ 383.31 Toronto Research Chemicals, S688825 | 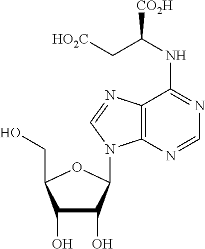 |
| Full name Short name CAS Number Molecular Formula Molecular Weight | 2-(2-((3-((2R,4S,5R)-4-hydroxy-5- (hydroxymethyl)tetrahydrofuran-2-yl)- 3,7-dihydropyrimido[2,1-i]purin-7- yl)oxy)cyclopropylidene)malonaldehyde (57) M3dA NA C₁₉H₁₉N₅O₆ 413.13 | 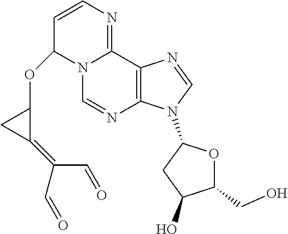 [SMILES: O=CC(C=O)=C1CC1OC1C=CN=C2c3ncn([C@H]4C[C@H](O)[C@@H](CO)O4)c3N=CN21] |

All exemplary compounds were shown to interact with MR1, elicited MR1-restricted T cell responses and/or stabilized MR1 expression on cells as evidenced by results obtained by the assay methods as shown in the examples.

### Examples

### Material and Methods

### Human Blood Samples

Blood and tissue specimens for T cell cloning, FACS analysis and antigen-presentation assays were obtained from the University Hospital Basel after informed consent, according to protocols EKNZ 2017-01888, which received ethical approval from the Swiss authorities (EKNZ, Ethics Committee North-West & Central Switzerland), and all patients and healthy donors consented in writing to the analysis of their samples.

### Cell Lines

The cell lines used as antigen-presenting cells (APCs) in this study are A375 (ATCC CRL-1619), THP-1 (ATCC TIB-202), A375-MR1 and THP1-MR1, previously generated and described (Lepore et al 2017). The HEL, Me67, Mel JUSO, H460, KMOE-2 and TF-1 tumor cell lines were cultured in RPMI-1640 supplemented with 10% FCS, 2 mM L-glutamine, 1 mM sodium pyruvate, 1×MEM NEAA and 50 μg/ml kanamycin (all from Bioconcept). The culture media for TF-1 cells was additionally supplemented with 10 ng/ml recombinant human GM-CSF (Peprotech). All human T cell clones were maintained in culture as previously described. A representative MAIT clone (MRC25) generated from blood of a healthy donor was previously characterized (Schmaler et al. (2018). Mucosal Immunology 11:1060-1070). Cells were free from Mycoplasma as evaluated by PCR analysis on DNA samples. When possible, cells were authenticated by staining with mAb for specific cell surface markers.

Lentiviral transductions were carried out as previously described. Transduced cells were selected by FACS sorting based on the expression of EGFP or mCherry reporters, or by 2 μg/mL puromycin resistance.

### Human Knockout Library Screening

A375-MR1-Cas9 cells generated using the previously described cell line and Lenti Cas9-Blast plasmid (Addgene) were transduced at 0.3 MOI by both part A and B of the pooled Human GeCKO v2 CRISPR library (Addgene), and subsequently selected by 2 μg/mL puromycin (Calbiochem, Cat #540411) for 96 hours. Eight biological replicates of the resulting APCs, each with 64-fold representation of each guide within the library underwent 4 consecutive rounds of killing by TC5A87 cells at a E:T of 2:1, following which surviving cells were expanded for 24 hours and DNA extracted using the NucleoSpin Tissue kit (Macherey-Nagel, Cat #740952). An additional 8 biological replicates were similarly prepared but did not undergo killing by TC5A87 to act as controls. Illumina libraries were prepared as previously described. Briefly, primers JScrispr1 and JScrispr3 were used to amplify genomic gRNA from the extracted gDNA and attach common Illumina primer handles for attaching sequencing library indexes. Additionally, the former primer inserts an 8-nt degenerate sequence immediately downstream of the Illumina read 1 start site, decreasing issues of sequencing low-complexity libraries. Each replicate was barcoded by a unique pair of Nextera indexes (Illumina, Cat #15055290) in a second step PCR performed as described in the Nextera DNA library preparation protocol (Illumina). The high-fidelity Advantage HF2 PCR kit (Takara, Cat #639123) was used in each of the PCR steps involved in preparing the sequencing libraries. Libraries were quantified using the BioAnalyser high sensitivity DNA kit (Agilent, Cat #5067-4626) and Qubit high-sensitivity dsDNA kit (ThermoFisher, Cat #Q32851) and pooled to form an equimolar sequencing library that was denatured and diluted to 1.2 μM with 20% PhiX v3 control library (Illumina, Cat #FC-110-3001) as described in the Illumina Denature and Dilution protocol (Illumina) before sequencing on a NextSeq500 using the High-output 150-cycle v2 kit (Illumina, discontinued product). Both sets of sequencing libraries were sequenced using a dual-indexed single-end protocol (131 cycles on read 1, 8 cycles on each barcode) to a depth of 25 million reads per replicate, ensuring that guides depleted after T-cell mediated killing may be detected.

Isolation and Culture of Primary Cells from Human Blood and Tissue Samples

MR1T cells were isolated from the peripheral blood of healthy individuals. After PBMC separation by density gradient centrifugation, T cells were purified by negative selection using EasySep Human T Cell Enrichment Kit and stimulated with irradiated (80 Gray) A375-MR1 cells (ratio 2:1) and antigen once a week for three weeks. Human rIL-2 (5 U/mL) was added at day +2 and +5 after each stimulation. Twelve days after the final stimulation, cells were washed and co-cultured overnight with A375-MR1 cells (ratio 2:1) in the presence or absence of antigens.

CD3⁺ CD69⁺ CD137^{high} cells were then FACS sorted and cloned by limiting dilution in the presence of phyto-haemagglutinin (1 μg/mL, Remel, Cat #30852801 HA16), human rIL-2 (100 U/mL,) and irradiated PBMC (5×10⁵ cells/mL). In some experiments, MR1T cells clones were isolated by limiting dilution of a FACS sorted CD3⁺, M₃ADE-MR1-tetramer⁺ cells from a T cell line generated through expansion of purified T cells with A375-β2mKO-MR1 cells pulsed with synthetic Ag. T cell clones were periodically re-stimulated following the same protocol. PBMCs were isolated from peripheral blood by density gradient centrifugation and frozen in liquid N₂ until use.

T cells, B cells, monocytes, myeloid dendritic cells (mDCs) and plasmacytoid dendritic cells (pDCs) were purified from PBMCs using immunomagnetic separation with kits indicated in Key Resource table, according to manufacturer's protocol.

Tissue biopsy samples derived from small cell lung tumors that were digested with media containing Accutase (Innovative Cell; Cat #AT-104), Collagenase IV 200 U/mL (Worthington; Cat #LS004189), DNAse I 0.5 mg/mL (Sigma-Aldrich Cat #D5025) and Hyaluronidase 50 mg/mL (Sigma; Cat #H6254) for 1 hour at 37° C. Digested material was passed through a 70 μM cell strainer and erythrocytes were lysed before being frozen and stored in liquid nitrogen. After thawing, TILs were rested for 2 days prior to co-culture with A375-p2mKO-MR1 cells in a ratio of 1:1 and in the presence of 50 μM M₃ADE. On day 5, human rIL-2 (5 U/mL) was added to the cultures for a further 5 days, and expansion in this manner was repeated 3 times. Cells were then stained with MR1-M₃ADE tetramer, anti-CD3, anti-CD4 and anti-CD8 mAbs, and tetramer positive cells were sorted into a bulk line before functional experiments.

### CRISPR-Cas9-Mediated Gene Disruption

Results obtained in the screening were confirmed by knock-out of selected genes in A375-MR1-Cas9 cells transduced with gRNAs different from the ones present in the library (Table 4). After lentiviral transduction and selection, A375-MR1-Cas9 cells were maintained for limited number of passages and used in activation assays as bulk population. THP-1 cells were cloned by limiting dilution and screened for GLO1 expression. Expression levels of target proteins was assessed by western blotting (FIG. 15). MR1 surface expression was evaluated by flow cytometry with APC-labeled mouse anti MR1 mAbs 26.5 (Biolegend) and APC-labeled mouse IgG2a (clone MOPC-173) as isotype control (FIG. 15).

The Antigen presentation ability of different cell lines was tested by stimulation of MAIT clone MRC25 after pulsing APCs 2 h at 37° C. with indicated concentrations of freshly-prepared 5-OP-RU (FIG. 15).

### TCR Gene Transfer

The TCRα and β functional cDNA from MR1T clones were cloned into a modified version of the Lenti expression vector (Addgene, Cat #52962). Endogenous TCR-deficient SKW-3 or J76 cells were transduced with virus particle-containing supernatants generated as previously described (Lepore et al. 2017). Transduced cells were selected by FACS sorting based on CD3 expression, when necessary.

### Preparation and Purification of Synthetic Antigens

Compounds M₃ADE, OPdA, M₁G, OPdC, M1dC, MGdA, MGG, m6,6A, io6A, m6t6A, Ar(p), M1dA, M3dA, ONEdA, m2,2G, Gr, CEdG, ONEdG, M1dG, N²OPdG, M2dG, m3U, m3Um, yW, OHyW, Psi, ONEdC, M3dC were synthesized and subsequently purified before use with cells. All other compounds were purchased from different vendors as indicated in Table 1.

### Synthesis of 8-(9H-purin-6-yl)-2-oxa-8-azabicyclo[3.3.1]nona-3,6-diene-4,6-dicarbaldehyde (M₃ADE) (13)

M₃ADE was synthesized as previously described (Stone et al., Chemical Research in Toxicology 3, 33-38 (1990)) with some modifications. 1,1,3,3-tetraethoxypropane (1.1 g, 5 mmol, 4.0 eq.) in aq. HCl (25 mL, 1 M) was stirred at 40° C. for 1 h. Subsequently, a solution of adenine (168.9 mg, 1.25 mmol, 1.0 eq.) in water (25 mL) was added. The mixture was adjusted to pH 4.0 with aq. NaOH (1 M) and stirred for 5 days at 37° C. M₃ADE was purified by solid phase extraction over Sep-Pak C18 2 g cartridges (Waters Corp., Milford, Mass.). Cartridges were preconditioned with 10 mL water and 10 mL Acetonitrile. Raw M₃ADE was washed with 20 mL water, 20 mL 10% Acetonitrile, then eluted with 20 mL 20% Acetonitrile.

M₃ADE HPLC purification was performed on a JASCO RHPLC system equipped with an MD-4010 Photo Diode Array detector. Semi-preparative HPLC purification was performed using a 250×10 mm 5 μM NUCLEODUR C18 Pyramid HPLC column at a temperature of 23° C. where the mobile phases A and B were water and 95% methanol in water, respectively. Separation was performed with a flow rate of 6 mL/min with a linear gradient of 0-50% B from 0 to 15 min, 50-100% B from 15 to 38 min, 100% B from 38 to 43 min, 100-0% B from 43 to 44 min and 0% B until 50 min. M₃ADE yield was 12.5 mg (42 μmol, 3.4%). Biologically active HPLC peaks were collected for mass spectrometric and NMR analyses.

¹H-NMR (600 MHz, D₂O, δ/ppm): 9.24 (s, 1H, H₂₁), 9.11 (s, 1H, H₁₉), 9.09 (s, 1H, H₁s), 8.61 (s, 1H, H₂), 8.38 (s, 1H, H₈), 7.64 (s, 1H, H₁₇), 7.33-7.29 (m, 1H, H₁₁), 4.09-4.07 (m, 1H, H₁₃), 2.21 (ddd, ²J_{H12a-H12b}=13.7 Hz, ³J_{H12a-H11}=2.8 Hz, ³J_{H12a-H13}=2.8 Hz, 1H, H₁₂a), 2.03 (ddd, ²J_{H12b-H12a}=13.7 Hz, ³J_{H12b-H11}=2.2 Hz, ³J_{H12b-H13}=2.2 Hz, 1H, H_{12b}).

¹³C-NMR (151 MHz, D₂O, extracted from HSQC and HMBC, δ/ppm): 193.2 (C₁₉), 192.2 (C₂₁), 166.5 (C₁₇), 154.3 (C₄), 152.7 (C₂), 150.3 (C₆), 149.3 (C₁₅), 144.8 (C₈), 126.2 (C₁₄), 125.7 (C₁₆), 121.8 (C₅), 79.9 (C₁₁), 25.0 (C₁₂), 17.4 (C₁₃).

HR-ESI-MS: calcd. for [M+Na]⁺ C₁₄H₁₁N5NaO₃ m/z=320.0754, found 320.0758.

### Synthesis of Pyrimido[1,2-a]purin-10(3H)-one (24) (M₁G, CAS 103408-45-3

M₁G was synthesized as previously described (Seto et al. Bulletin of the Chemical Society of Japan 58, 3431-3435 (1985).; Hadley and Draper, Lipids 25, 82 (1990).) with some modifications. 1,1,3,3-Tetraethoxypropane (1.4 g, 6.25 mmol, 5.0 eq.) in aq. HCl (25 mL, 1 M) was stirred at 40° C. for 1 hour. Subsequently, a solution of guanine (188.9 mg, 1.25 mmol, 1.0 eq.) in aq. HCl (25 mL, 1 M) was slowly added. The mixture was stirred at 40° C. for 1 h and then kept at 4° C. for 16 h. The precipitate was washed 3 times with absolute ethanol at 2000×g for 10 min. The raw M₁G was extracted 3 times from the precipitate with 65° C. water. The combined extracts were filtered with 0.22 μm filter. The mixture was adjusted to pH 7.0 with aq. NaOH (1 M).

HPLC analysis for M₁G was performed on a JASCO RHPLC system. Semi-preparative HPLC purification was performed using a 250/10 NUCLEODUR C18 Pyramid HPLC column with a column temperature of 23° C. Solvent A was Milli-Q water, solvent B consisted of 95% methanol and 5% Milli-Q water. The total run was 55 min with a flow rate of 6 mL/min. The initial mobile phase was 100% Solvent A for 10 min. Solvent B increased linearly until the gradient reached 80% Solvent A and 20% Solvent B at 40 min. Solvent B was increased linearly again until it was briefly 100% at 41 min. Isocratic flow at 100% B for 5 min, a linear gradient to 100% Solvent A for 1 min and continuous for 8 min. Biologically active HPLC peaks were collected for mass spectrometric and NMR analyses (12.5 mg, 66.8 μmol, 5.3%).

¹H-NMR (600 MHz, D₂O, δ/ppm): 9.31 (d, ³J_{H13-H12}=7.2 Hz, 1H, H₁₃), 8.97 (dd, ³J_{H11-H12}=4.1 Hz, ⁴J_{H11-H13}=2.0 Hz, 1H, H₁₁), 8.22 (s, 1H, H₈), 7.30 (dd, ³J_{H12-H13}=7.2 Hz, ³J_{H12-H11}=4.2 Hz, 1H, H₁₂).

¹³C-NMR (151 MHz, D₂O, extracted from HSQC and HMBC, δ/ppm): 162.9 (C₁₁), 154.4 (C₄), 154.1 (C₆), 149.9 (C₂), 146.0 (C₈), 138.4 (C₁₃), 116.7 (C₅), 112.0 (C₁₂).

HR-ESI-MS: calcd. for [M+H]+C₈H₆N₅O m/z=188.0567, found 188.0571.

### Synthesis of N⁶-(3-Oxo-1-propenyl)-2′-deoxyadenosine (12) (OPdA, CAS 178427-43-5)

OPdA was synthesized as previously described (Szekely et al., Nucleosides, Nucleotides and Nucleic Acids 27, 103-109 (2008)) with some modifications. 2′-deoxyadenosine monohydrate (219 mg, 0.813 mmol, 1 eq.) was dissolved in 2 mL anhydrous dimethyl sulfoxide under argon atmosphere. Propargyl aldehyde (12 μl, 11.0 mg, 0.203 mmol, 0.25 eq.) was added to the stirred solution and additional propargyl aldehyde (1.25 eq.) was added over a 72-h period. The reaction mixture was filtered and purified by preparative HPLC on a Shimadzu LC system (LC-20AT prominence liquid chromatograph, with an SPD-20A prominence UV/VIS detector (A=254 and 280 nm). Preparative HPLC purification was performed using a Reprosil-Pur 120 ODS 3, 5 μM, 150×20 mm column, where the mobile phases A and B were water and 90% acetonitrile in water, respectively. Separation was performed with a flow rate of 9 mL/min with a linear gradient of 1-30% B from 5 to 15 min, 30-100% B from 15 to 17 min, 100% B from 17 to 21 min, 100-0% B from 21 to 22 min and 1% B until 25 min. Analytical HPLC was performed with a LC-20AD prominence liquid chromatograph combined with a Shimadzu LCMS-2020 liquid chromatograph mass spectrometer. Biologically active HPLC peaks were collected for mass spectrometric and NMR analyses. The OPdA yield was 13.5 mg (44.0 μmol, 5.4%).

¹H-NMR (500 MHz, D₂O, δ/ppm): 9.21 (d, ³J_{H13-H12}=8.7 Hz, 1H, H₁₃), 8.49 (d, ³J_{H11-H12}=13.5 Hz, 1H, H₁₁), 8.43 (s, 1H, H₈), 8.38 (s, 1H, H₂), 6.45 (dd, ³J_{H1′-H2′a}=6.8 Hz, ³J_{H1′-H2′b}=6.8 Hz, 1H, H_{1′}), 5.89 (dd, ³J_{H12-H11}=13.5, ³J_{H12-H13}=8.7 Hz, 1H, H₁₂), 4.65 (ddd, ³J_{H3′-H2′a}=6.1 Hz, ³J_{H3′-H2′b}=3.5 Hz, ³J_{H3′-H4′}=3.5 Hz, 1H, H_{3′}), 4.19 (ddd, ³J_{H4′-H5′b}=3.8 Hz, ³J_{H4′-H5′a}=3.5 Hz, ³J_{H4′-H3}′=3.5 Hz, 1H, H_{4′}), 3.86 (dd, ²J_{H5′a-H5′b}=12.5 Hz, ³J_{H5′a-H4′}=3.4 Hz, 1H, H_{5′a}), 3.80 (dd, ²J_{H5′b-H5′a}=12.6 Hz, ³J_{H5′b-H4}′=4.3 Hz, 1H, H_{5′b}), 2.80 (ddd, ²J_{H2′a-H2′b}=13.7 Hz, ³J_{H2′a-H1′}=7.1 Hz, ³J_{H2′a-H3′}=6.4 Hz, 1H, H_{2′a}), 2.58 (ddd, ²J_{H2′b-H2′a}=14.0 Hz, ³J_{H2′b-H1}=6.3 Hz, ³J_{H2′b-H3′}=3.5 Hz, 1H, H_{2′b}).

¹³C-NMR (126 MHz, D₂O, extracted from HSQC and HMBC, δ/ppm): 195.7 (C₁₃), 151.9 (C₂), 151.1 (C₁₁), 150.6 (C₄), 148.9 (C₆), 142.6 (C₅), 120.8 (C₅), 111.1 (C₁₂), 87.5 (C_{4′}), 84.7 (C_{1′}), 71.1 (C_{3′}), 61.6 (C_{5′}), 39.1 (C_{2′}).

HR-ESI-MS: calcd. for [M+Na]⁺ C₁₃H₁₅N5NaO₄ m/z=328.1016, found 328.1020.

### Synthesis of N⁴-(3-Oxo-1-propenyl)-2′-deoxycytidine (38) (OPdC, CAS 129124-79-4

OPdC was synthesized as previously described (Szekely et al. ibid.) with some modifications. 2′-deoxycytidine (185 mg, 0.813 mmol, 1 eq.) was dissolved in 2 mL anhydrous dimethyl sulfoxide under argon atmosphere. Propargyl aldehyde (12.0 μL, 11.0 mg, 0.203 mmol, 0.25 eq.) was added to the stirred solution and additional propargyl aldehyde (1.25 eq.) was added over a 72-hour period. The reaction mixture was filtered and purified by prep.

HPLC purification was performed as described for OPdA. The OPdC yield was 7.00 mg (25.0 μmol, 3.1%). Biologically active HPLC peaks were collected for mass spectrometric and NMR analyses.

¹H-NMR (500 MHz, D₂O, δ/ppm): 9.31 (d, ³J_{H10-H9}=8.6 Hz, 1H, H₁₀), 8.29 (d, ³J_{H8-H9}=13.7 Hz, 1H, H₈), 8.19 (d, ³J_{H6-H5}=7.4 Hz, 1H, H₆), 6.28 (d, ³J_{H5-H6}=7.4 Hz, 1H, H₅), 6.24 (dd, ³J_{H1′-H2′a}=6.1 Hz, ³J_{H1′-H2′b}=6.1 Hz, 1H, H_{1′}), 5.91 (dd, ³J_{H9-H8}=13.7 Hz, ³J_{H9-H10}=8.6 Hz, 1H, H₉), 4.43 (ddd, ³J_{H3′-H2′a}=6.4 Hz, ³J_{H3′-H2′b}=4.3 Hz, ³J_{H3′-H4′}=4.3 Hz, 1H, H_{3′}), 4.12 (ddd, ³J_{H4′-H5′b}=4.8 Hz, ³J_{H4′-H5′a}=4.1 Hz, ³J_{H4′-H3′}=4.1 Hz, 1H, H_{4′}), 3.87 (dd, ²J_{H5′a-H5′b}=12.5 Hz, ³J_{H5′a-H4′}=3.5 Hz, 1H, H_{5′a}), 3.77 (dd, ²J_{H5′b-H5′a}=12.5 Hz, ³J_{H5′b-H4}′=5.3 Hz, 1H, H_{5′b}), 2.55 (ddd, ²J_{H2′b-H2′a}=14.1 Hz, ³J_{H2′b-H1′}=6.3 Hz, ³J_{H2′b-H3′}=4.3 Hz, 1H, H_{2′b}), 2′0.32 (ddd, ²J_{H2′a-H2′b}=14.2 Hz, ³J_{H2′a-H1′}=6.5 Hz, ³J_{H2′a-H3′}=6.5 Hz, 1H, H_{2′a}).

¹³C-NMR (126 MHz, D₂O, extracted from HSQC and HMBC, δ/ppm): 196.2 (C₁), 161.5 (C₄), 156.8 (C₂), 149.6 (C₈), 144.3 (C₆), 112.1 (C₉), 96.8 (C₅), 87.1 (C_{1′}), 87.1 (C_{4′}), 70.3 (C_{3′}), 61.1 (C_{5′}), 39.8 (C_{2′}).

HR-ESI-MS: calcd. for [M+Na]⁺ C₁₂H₁₅N₃NaO₅ m/z=304.0904, found 304.0902.

M₁dC was produced by mixing 2′-deoxycytidine (25 mM, Sigma, Cat #D3897) with malonaldehyde tetrabutylammonium salt (100 mM). The mixture was incubated for 18 h at 70° C. under 400 rpm shaking. M₁dC crude compound preparation was subjected to Solid Phase Extraction as above. M₁dC was eluted with 20% acetonitrile.

Further HPLC purification was performed by reversed-phase HPLC with a C18 Pyramid column (Macherey-Nagel, Cat #762204.40) as follows. Mobile phase A: deionized water; mobile phase B: 95% methanol in deionized water. Flow rate 1.25 mL/min. Elution gradient: time 0 min, A 100%; time 1 min, A 98%; time 43 min A 50%; time 46 min A 50%; time 47 min A 100%; time 56 min A 100%. Biologically active HPLC-separated compounds were collected for mass spectrometric and NMR analyses.

MGG was produced by mixing guanosine (100 mM, Sigma, Cat #G6752) with methylglyoxal solution (100 mM) in DMSO (33.3%, v/v in water, Sigma Cat #D4540). The mixture was incubated for 2 h at 70° C. under 400 rpm shaking. Further HPLC purification was performed by reversed-phase HPLC with a C18 Pyramid column (Macherey-Nagel, Cat #762272.100) as follows. Mobile phase A: deionized water; mobile phase B: 95% methanol in deionized water. Flow rate 5 mL/min. Elution gradient: time 0 min, A 97%; time 2.5 min, A 97%; time 30 min A 87%; time 32.5 min A 0%; time 37.5 min A 0%; time 40 min A 100%, time 52.5 min A 100%. Biologically active HPLC-separated compounds were collected for mass spectrometric and NMR analyses.

### MS and NMR Analysis

Unless otherwise stated, chemicals were used as received without further purification. NMR analysis of all of the antigens was performed at 298 K on a Bruker Avance III NMR spectrometer operating at 500 MHz proton frequency equipped with a BBFO probehead or on a Bruker Avance III HD NMR spectrometer operating at 600 MHz proton frequency equipped with a cryogenic QCI-F probe. Standard pulse sequences were used for cosy, tocsy, noesy, hsqc, hmqc and hmbc 2D-NMR experiments and the spectra were processed using the topspin 4.0 software package. All new compounds were fully characterized by means of 2D-NMR and HiRes-ESI-MS. For all compounds ¹H- and ¹H-¹³C-HSQC spectra, as well as experimental and calculated HiRes-ESI-MS spectra were obtained (not shown) HRMS spectra were measured on a Bruker MaXis 4G high resolution ESI Mass Spectrometer in direct injection mode using methanol containing 0.1% v/v formic acid.

### Cell Surface MR1 Upregulation

THP-1 cells (10⁵ cells/well) were tested for MR1 surface expression after incubation with or without synthetic compounds: M₃ADE (1 μM), OPdA (100 μM), M₁G (13 μM) and OPdC (100 μM) for 6 h at 37° C. Ac-6-FP (acetyl-6-formylpterin, 100 μM) (Schircks Laboratories, Cat #11.418) was used as positive control for MR1 surface upregulation. The cells were stained with an anti-human-MR1-APC mAb (clone 26.5) or with APC-labeled mouse IgG2a, k isotype control antibodies for 20 min at 4° C., then washed and analyzed by flow cytometry. For each condition, net MFI was calculated subtracting isotype MFI from anti-MR1 MFI and fold change of cells treated with synthetic molecules over cells treated with vehicle was calculated.

Activation Assay with Living or Fixed APCs

MR1T cells (5×10⁴/well unless otherwise indicated) were co-cultured with the indicated APCs (10⁵ cells per well unless otherwise indicated) for 18 h in 120 μL volume in triplicate. In some experiments, anti-MR1 mAbs (clone 26.5, purified and endotoxin-free mouse IgG2a, (Lepore et al., 2014)) or mouse IgG2a isotype control mAbs (LEAF, Biolegend, Cat #401504) (both at 30 μg/mL) were added and incubated for 30 min at 37° C. prior to the addition of T cells.

When nucleobases, nucleosides or nucleotides (all 250 μM) and synthetic compounds M₃ADE, OPdA, M₁G or OPdC were used to stimulate T cells, the THP-1 cells (10⁵/well) were cultured 2 h with the indicated molecules or medium only, prior to T-cell addition. When a single concentration of synthetic antigens was used, 100 μM OPdA, 100 μM OPdC and 13 μM M₁G were used for all clones, while 100 μM M₃ADE was used for all clones except DGB129, AC1A4, AC1B76 and AVA46 for which 1 μM was used.

In experiments with mycophenolic acid (10 μM), EHNA (25 μM), S-p-bromobenzylglutathione cyclopentyl diester (BBG, 20 μM) (all from Sigma-Aldrich), THP-1 cells (1×10⁶/mL) were treated with the indicated concentrations of drugs in complete medium at 37° C. for 18 h before being washed twice with PBS, counted and used for T-cell activation. In experiments where THP-1 cells (1×10⁶/mL) were treated with doxorubicin (75 nM) and paclitaxel (5 μM) (both from Sigma-Aldrich), the cells were incubated at 37° C. for 18 h before being washed twice with PBS, counted, and incubated for 2 h with vehicle or 150 μM dAdo and 150 μM guanosine (Sigma-Aldrich) prior to T-cell addition. In experiments where fixed A375-MR1 cells were used to activate MR1T cells, APCs (4×10⁵/mL) were treated with apocynin (APO, 100 μM), L-glutathione reduced (GSH, 4 mM), N-acetyl cysteine (NAC, 4 mM), L-buthionine-sulfoximine (BSO, 400 μM) mercaptosuccinic acid (MSA, 3.3 μM), ML-210 (6 μM) or 1S,3R-RSL3 (RSL3, 1 μM), hydralazine hydrochloride (100 μM) or aminoguanidine hemisulfate salt (5 mM) for 18 h at 37° C. before being washed twice with PBS, fixed with glutaraldehyde, counted and used for MR1T cell stimulation.

In some experiments, MAIT cells were stimulated by APCs pulsed 3 h with 5-OP-RU as previously described or with 30 μM 6,7-dimethyl-8-ribityllumazine (Cayman Chemical Cat #23370).

To confirm that drugs reducing MR1T stimulation do not affect MR1 presentation ability, A375-MR1 cells treated with different molecules were collected before fixation and used to stimulate the MAIT clone MRC25 after pulsing for 2 h at 37° C. with the indicated concentrations of freshly-prepared 5-OP-RU or 6,7-dimethyl-8-ribityllumazine (Cayman Chemicals).

Activation Assay with Plate-Bound Soluble MR1

Recombinant human P2m-MR1-Fc was produced in CHO-K1 cells as previously described (Lepore et al., 2017) and 4 μg/mL were coated onto 96 wells plates (Nunc, Cat #439454) for 18 h at 4° C. Plate-bound MR1 was then washed twice with wash buffer (150 mM NaCl, 20 mM Tris and 2% Glycerol, pH 5.6) to remove bound antigens. Then, the synthetic antigens (M₃ADE, OPdA, M₁G and OPdC) were added at the indicated concentrations and incubated for 6 h at room temperature (RT). Unbound antigens were washed twice with PBS before the addition of excess PBS. In some experiments, bacteria-produced and refolded MR1-M₃ADE protein was serially diluted in PBS and added to a high protein binding plate (Nunc, Cat #439454) for 2 h at 37° C. then washed twice and used in the stimulation assay. Indicated MR1T cell clones (105/100 μl/well) were added and supernatants were collected after 18 h. Released cytokines were detected by ELISA. Recombinant human P2m-MR1-Fc was produced in CHO-K1 cells as previously described and 4 μg/mL were coated onto 96 wells plates. Antigens produced by CHO-K1 cells were removed by washing twice with wash buffer (150 mM NaCl, 20 mM Tris and 2% Glycerol, pH 5.6). Synthetic antigens were diluted in wash buffer and incubated 3 h at RT. Antigens were washed out with wash buffer before the addition of MR1T cell clones (10⁵/well). Supernatants were collected after 18 h for cytokine analysis by ELISA.

### Cytokine Analysis

The following human cytokines were assessed by ELISA using specific mAbs: GM-CSF (purified clone BVD2-23B6 and biotinylated clone BVD2-21C11, Biolegend Cat #502202 and 502304, respectively), IFN-γ (purified clone MD-1 and biotinylated clone 4S.B3, Biolegend Cat #507502 and 502504, respectively), IL-13 (purified clone JES10-5A2 and biotinylated clone SB126d, SouthernBiotech Cat #10125-01 and 15930-08, respectively).

### MR1 Protein Production and Tetramerization

Soluble recombinant MR1 monomers were generated as previously described (Kjer-Nielsen, L., et al. (2012). Nature 491(7426): 717-723). Briefly, nucleotide sequences coding for the soluble portions of the mature human MR1 (GenBank accession number NM_001531) and mature human P2m (GenBank accession number NM_004048.3) were cloned into the bacterial expression vector pET23d (Novagen, Cat #69748-3). Transformed E. coli BL21(DE3)pLysS were then grown to ODeoonm 0.4-0.6 before induction with 0.6M isopropyl β-D-1-thiogalactopyranoside (Sigma-Aldrich, Cat #10724815001). After 4 h of further culture, cells were lysed and inclusion bodies were cleaned, purified and fully denatured in 8M Urea, 10 mM EDTA, 0.1 mM DTT for subsequent storage at −80° C.

For protein refolding, MR1 heavy chain (4 mM), β2m (2 mM) and compound (15 mM) were added to 1 L refolding buffer containing 0.4M L-arginine, 100 mM Tris pH 8.0, 2 mM EDTA that was cooled to 4° C. and supplemented with 5 mM reduced glutathione and 0.5 mM oxidized glutathione immediately beforehand. After 3 days, the refold mix was concentrated down to 1 mL and the refolded compound-bearing MR1 was purified by HPLC using Superdex 75 10/300 GL (GE Healthcare, Cat #17517401) and MonoQ 5/50 (GE Healthcare, Cat #17516601) columns.

Correct protein conformation was confirmed by performing a plate-bound activation of the MR1T cell clone DGB129. Refolded MR1-compound protein was serially diluted in PBS (1.5-100 μg/mL) and added to a high-protein-binding plate (Nunc, Cat #439454) for 2 h at 37° C. The wells were washed thoroughly with PBS before addition of 5×10⁴ cells that were left overnight at 37° C. IL-13 ELISA was then used as an activation readout. The functional monomer was then biotinylated using BirA-500 biotinylation kit (Avidity, Cat #Bulk BirA) overnight at 4° C. Excess biotin was removed using S75 10/30 (GE Healthcare, 29148721) gel filtration before tetramerization with phycoerythrin (PE) streptavidin (Prozyme, Cat #P JRS25) in a 4:1 molar ratio. As control MR1 tetramers, human MR1-5-OP-RU and human MR1-6-FP labelled with APC, PE or AlexaFluor488 were used (The MR1 tetramer technology was developed jointly by Dr. James McCluskey, Dr. Jamie Rossjohn, and Dr. David Fairlie, and the material was produced by the NIH Tetramer Core Facility as permitted to be distributed by the University of Melbourne).

### Immunofluorescence Staining

Cell surface labeling was performed using standard protocols. Intracellular labeling was performed using the True-Nuclear™ Transcription Factor Buffer Set (Biolegend, Cat #424401) according to the manufacturers' instructions. All mAbs for staining were titrated on appropriate cells before use. Biotinylated mAbs were revealed with streptavidin-PE (Biolegend, Cat #405204), -Alexa Fluor 488 (Biolegend, Cat #405235), or -Brilliant violet 421 (Biolegend, Cat #405226) all at 2 μg/mL.

When staining with tetramers, the cells were pre-treated with Dasatinib (50 nM, Sigma-Aldrich, Cat #CDS023389) for 30 min at 37° C. before first adding anti-CD8 mAbs (Biolegend, clone RP8-TA BV711) for 20 min at room temperature (RT), then 2.5 μg/mL tetramer for a further 20 min at RT without washing. All remaining mAbs were then added for a further 20 min at RT without washing. The cells were then washed in PBS before acquisition at the flow cytometer.

Samples were acquired on LSR Fortessa flow cytometer with the FACS Diva software (Becton Dickinson). Cell sorting experiments were performed using an Influx or FACSaria (Becton Dickinson). Dead cells and doublets were excluded on the basis of forward scatter area and width, side scatter, and DAPI (Sigma) or Live/Dead (Thermo Fisher Scientific) staining, as depicted in FIG. 6. When PBMCs were analyzed, CD14⁺ and CD19⁺ cells were excluded by staining with fluorescent antibodies detected in the same channel as the Live/Dead staining. All data was analyzed using FlowJo (LLC).

### ROS Production Measurement

CM-H₂DCFDA (Thermo Fisher Scientific) was used to assess ROS production in cell upon cell treatment with Doxorubicin and Paclitaxel. THP-1 cells (107/mL) were labelled with 10 μM CM-H₂DCFDA for 30 min at 37° C. in the dark, then washed with PBS and resuspended in complete medium. 10⁵ cells were seeded per well and treated with 75 nM Doxorubicin, 5 μM Paclitaxel or vehicle for 18 h at 37° C. Phorbol 12-myristate 13-acetate (PMA, 50 ng/mL) was used as positive control.

### Quantification and Statistical Analysis

sgRNA-Seq Data Processing and Analysis

Raw sequencing data was demultiplexed using bcl2fastq (v2.17.1.14) and read quality checked using FastQC (v0.11.4). Reads were then trimmed to remove the homologous regions flanking the sgRNA sequences using Trimmomatic v0.36 using options HEADCROP:42 CROP:20. These trimmed reads were again passed through FastQC to check that average phred33 quality in the sgRNA sequences was >30. These reads were aligned to a GeCKO v2 sgRNA reference index using Bowtie2 (v2.2.9) with options—very-sensitive-local. Read counts were then extracted from the resulting SAM files using custom perl script map_count.pl (Cox, M. available on request) and imported into R (R Development Core Team, 2018) for analysis using edgeR.

Inequality of sgRNA activity in the GeCKO library hinders hit-selection using rank-based methodology, thus differential enrichment analysis was performed with the edgeR package (Dai et al., 2014) using the GLM Robust method to estimate dispersions, after removing guides targeting known essential genes. The level of random enrichment and depletion of guides was estimated using the log 2 fold-change in the top and bottom 1% of negative control guides in the GeCKO library. Thus, guides with an FDR<0.05 and log 2 fold-change greater or lower than the top and bottom 1% of negative control guides, respectively, were said to be significantly enriched or depleted by the inventors screen. GO-term and KEGG-pathway enrichment analysis was performed using a binomial test on the significant unique gene-targets identified in the differential enrichment analysis {Carlson, 2016 #2707}. Genes were annotated using biomaRt version 2.42.0.

### CRISPR Screen Analysis

Data analysis, statistical tests and visualization were conducted in R and GraphPad Prism. After removing guides targeting known essential genes, differential enrichment analysis of CRISPR sequence data was performed using edgeR (v3.24) using the GLM Robust method to estimate dispersions. The level of random enrichment and depletion of guides was estimated using the log 2 fold-change in the top and bottom 1% of negative control guides in the GeCKO library. Thus, guides with FDR<0.05 and log 2 fold-change greater or lower than the top and bottom 1% of negative control guides, respectively, were said to be significantly enriched or depleted by the inventors screen. GO-term and KEGG-pathway enrichment analysis was performed using a binomial test on the significant unique gene-targets identified in the differential enrichment analysis. Genes were annotated using biomaRt version 2.42.0.

Statistical analysis was performed with Prism (GraphPad Software, Inc.) using multiple t-test, One- or Two-way Anova as indicated for each assay in Fig.legends.

A p value<0.05 was considered statistically significant. *p<0.05, **p 0.01, ***p 0.005.

### Results

### Compounds Stimulating MR1T Cells

The inventors' previous results showed that MR1T cells recognize MR1 molecules complexed with ligands present in tumor cells. The purification of cellular extracts of THP-1 cells, led them to the identification of compounds which can be defined as modified nucleobase and nucleobase adducts.

The inventors screened commercially available compounds using three types of biological assays. All three assays are based on the capacity of the compounds to bind MR1 and i) to modulate surface expression levels of MR1, ii) to activate in a specific manner at least one MR1T cell clone, or iii) to compete with the stimulatory compounds, thus affecting the response of MR1T cell clones. The biologically active compounds are reported in Table 1.

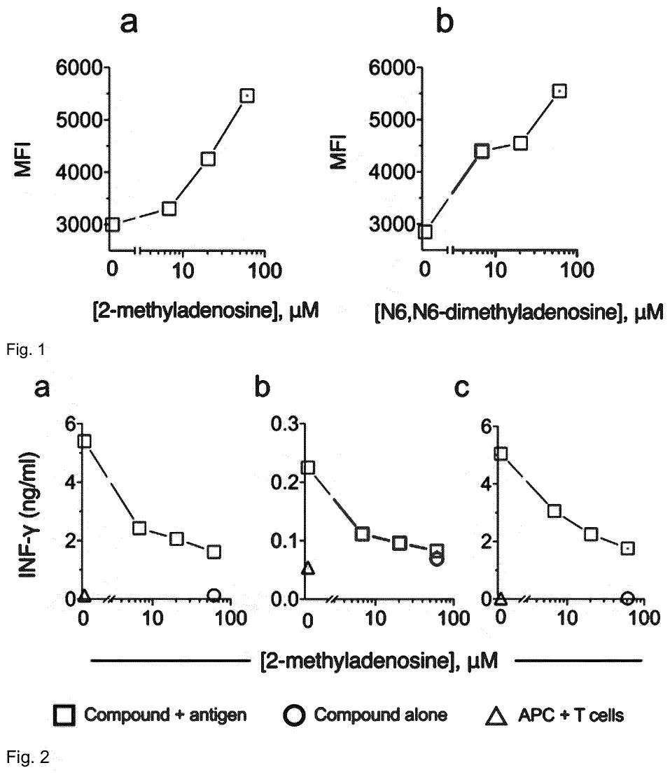

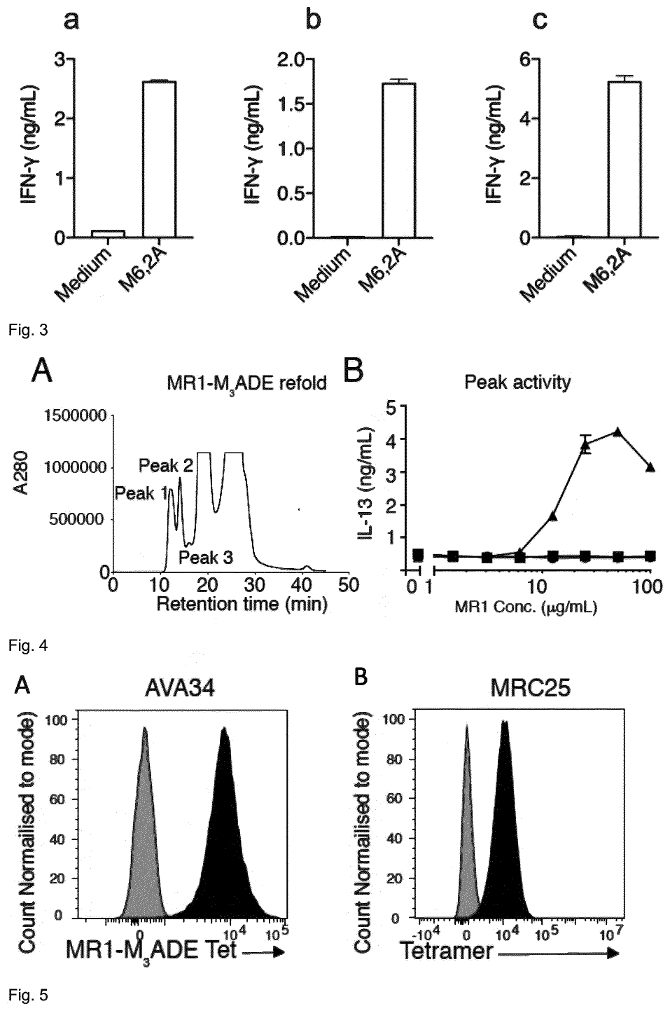

Examples of compound reactivity for each of the three functional assays indicated above, are illustrated in FIGS. 1 to 20.

### Detection and Sorting of MR1T Cells by MR1 Tetramer Staining

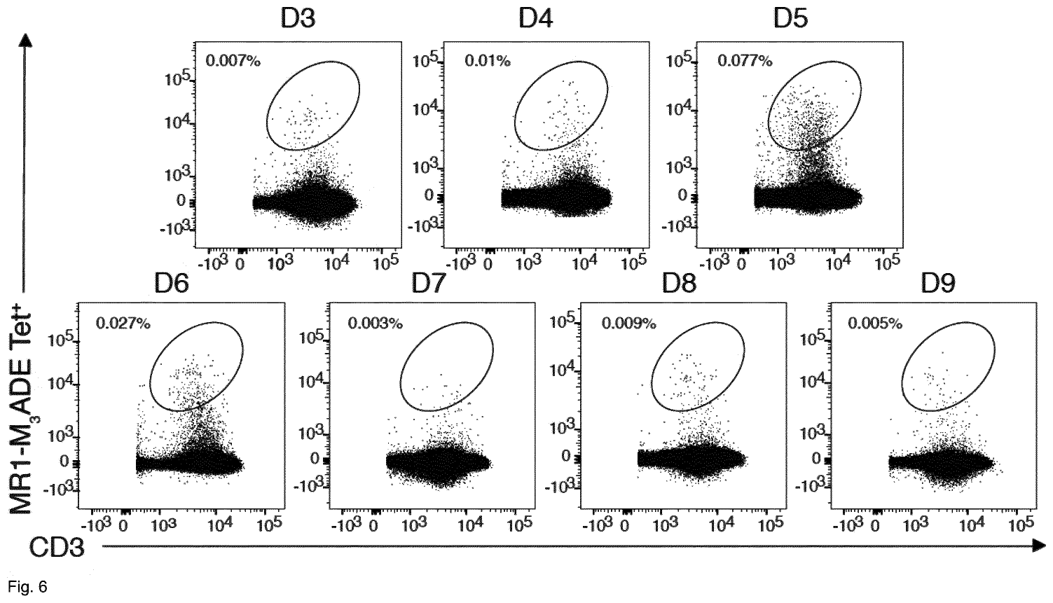

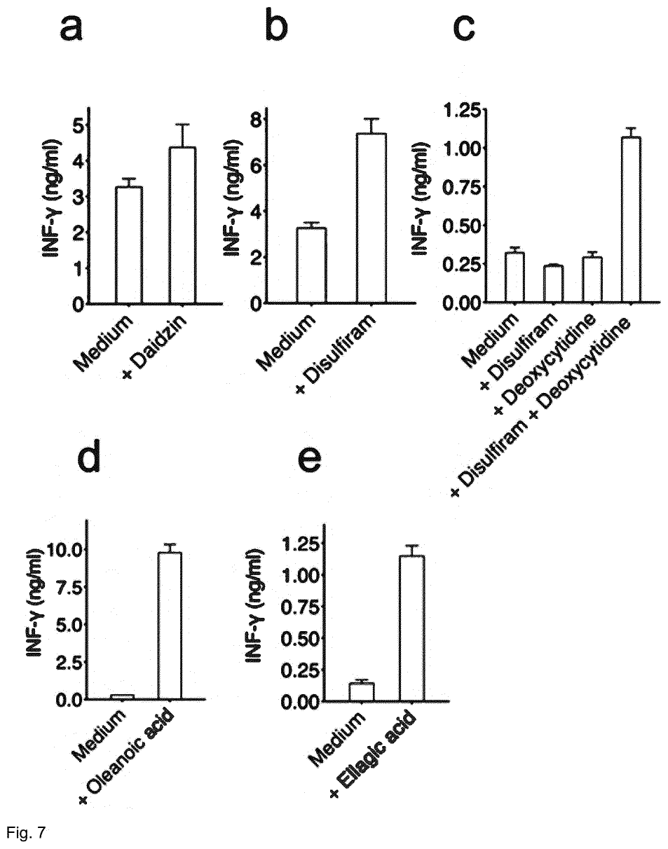

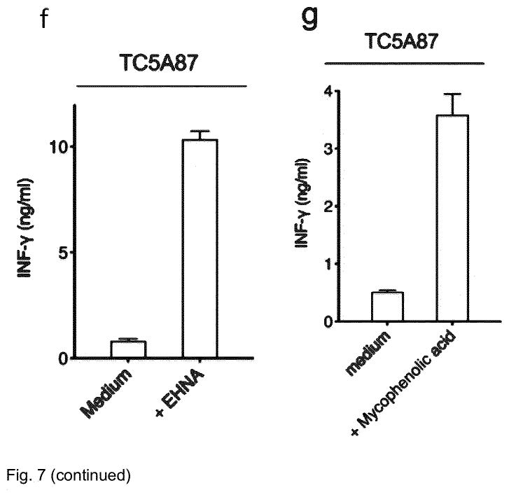

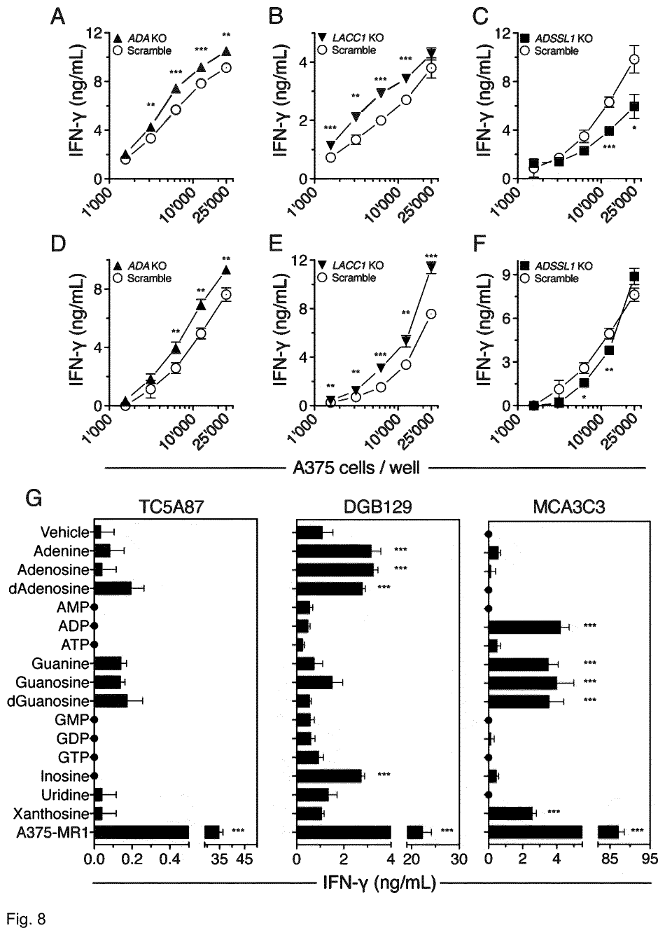

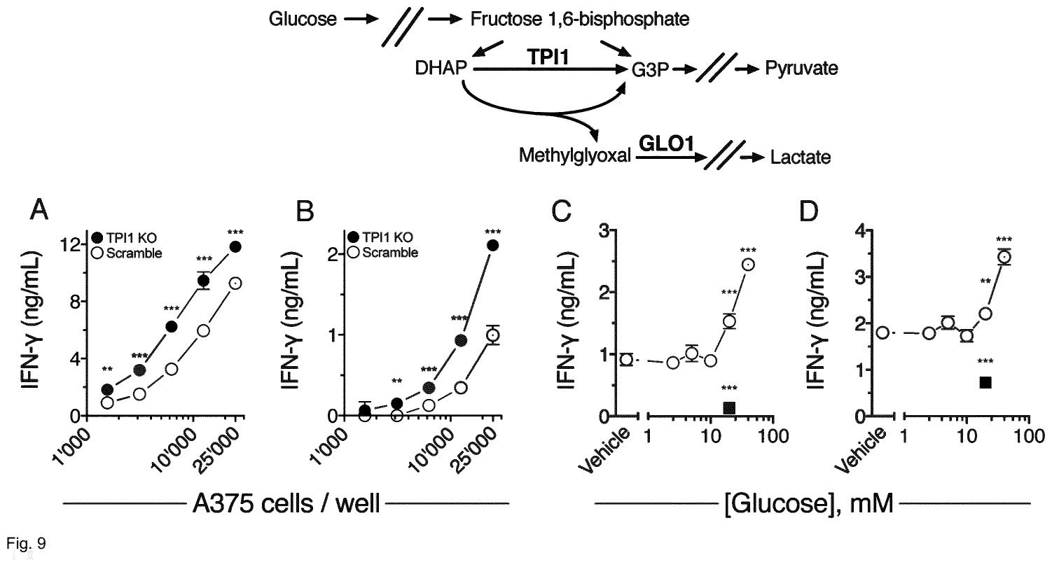

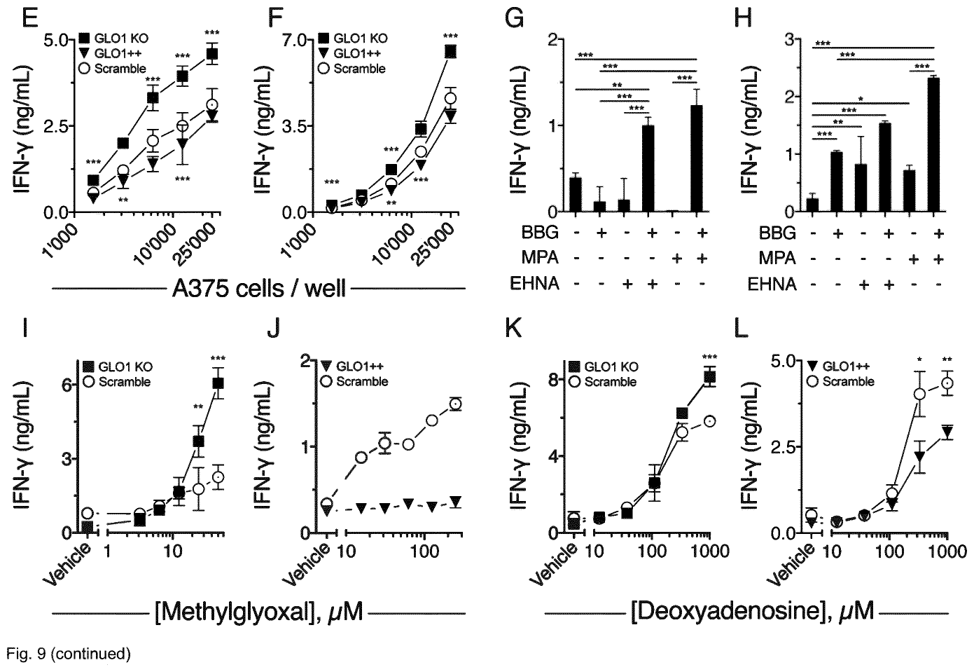

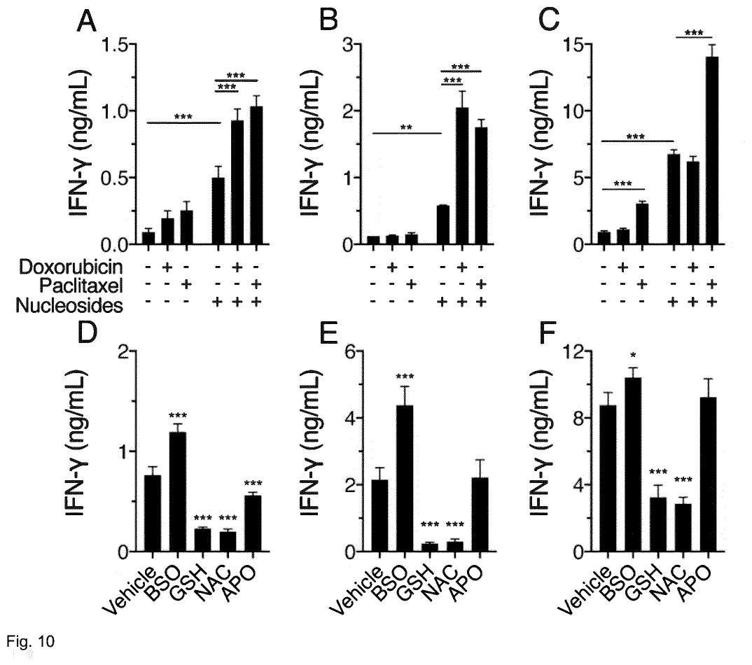

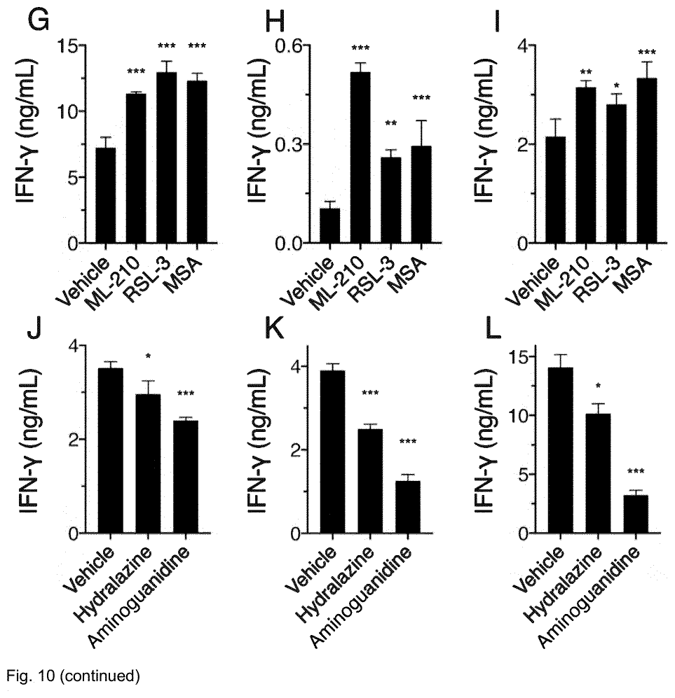

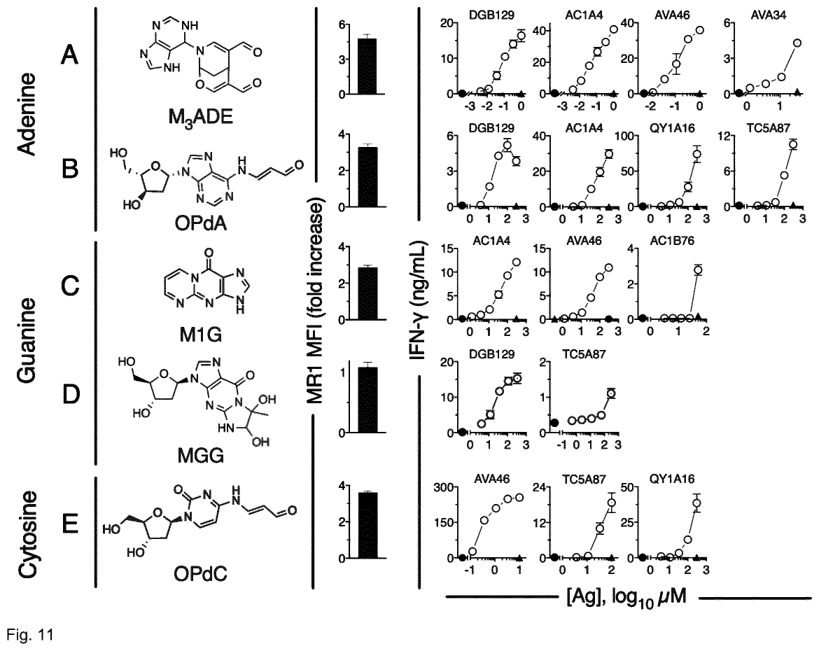

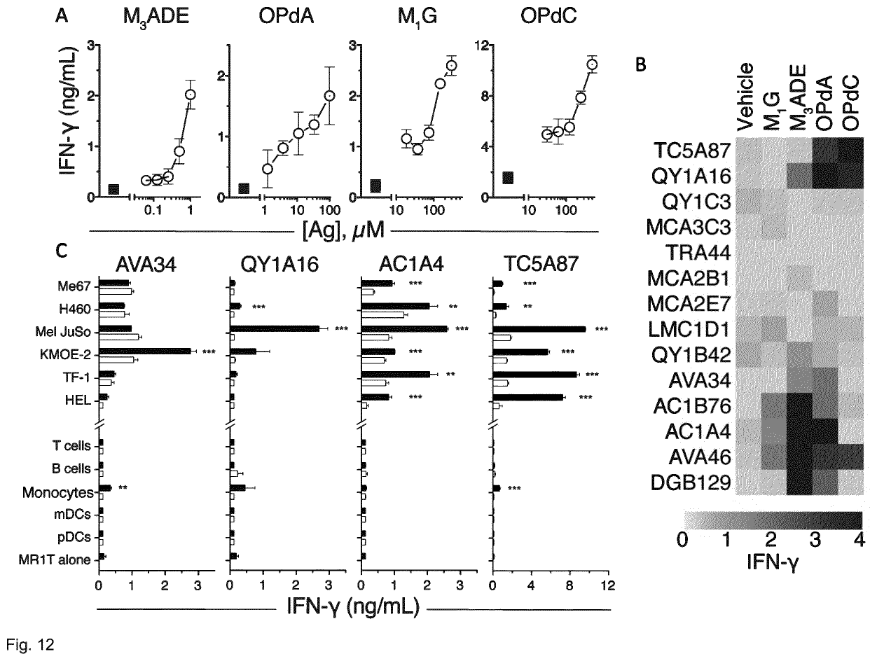

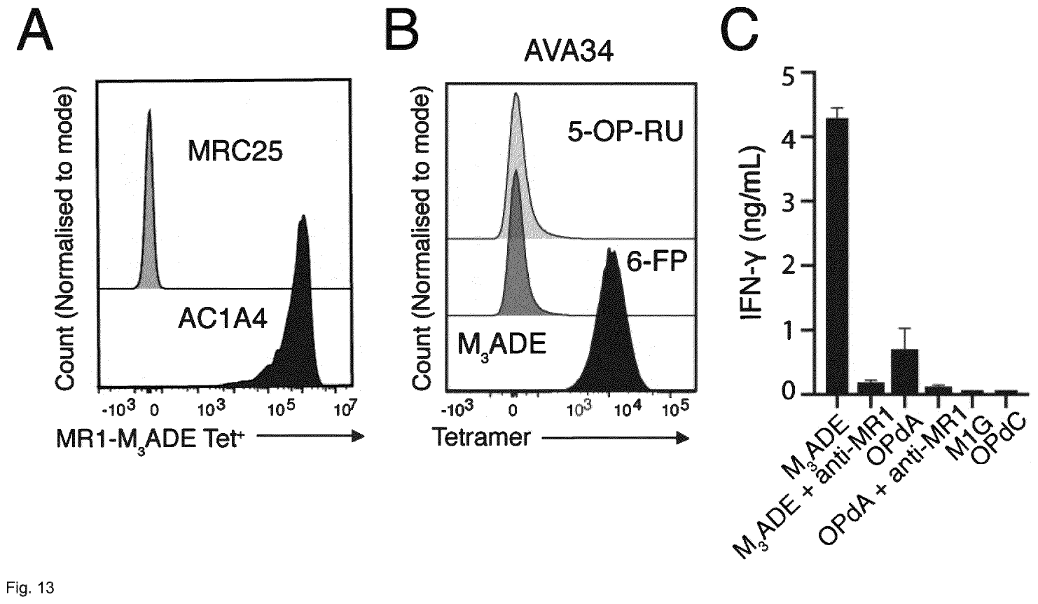

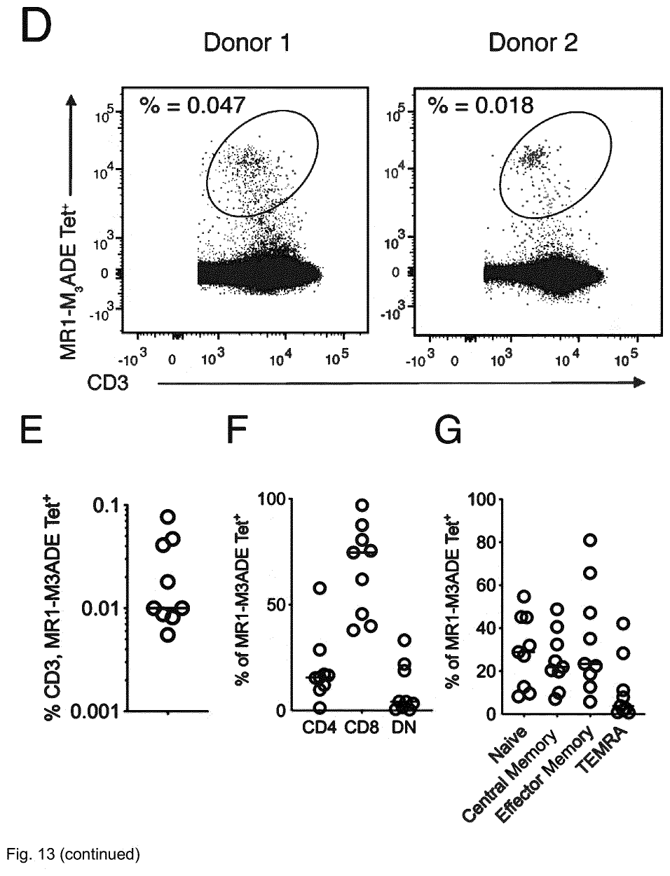

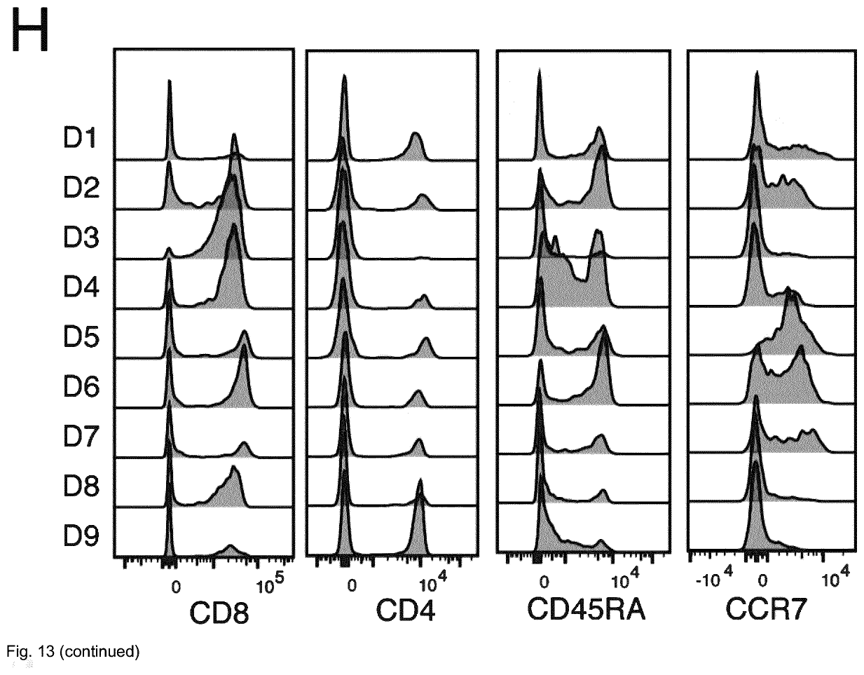

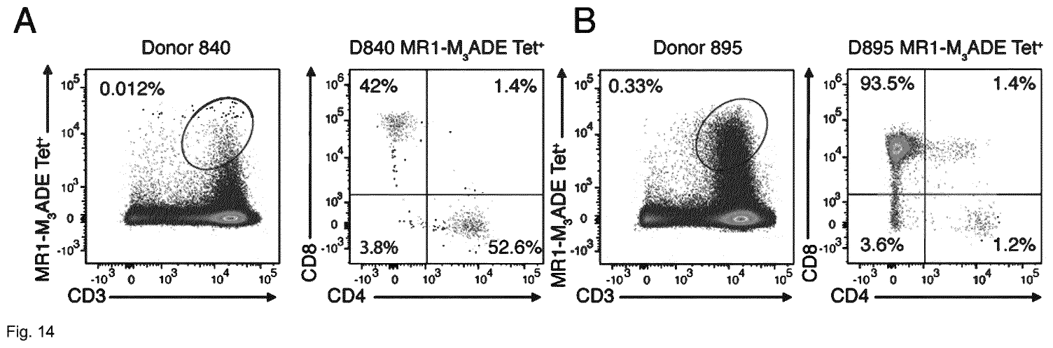

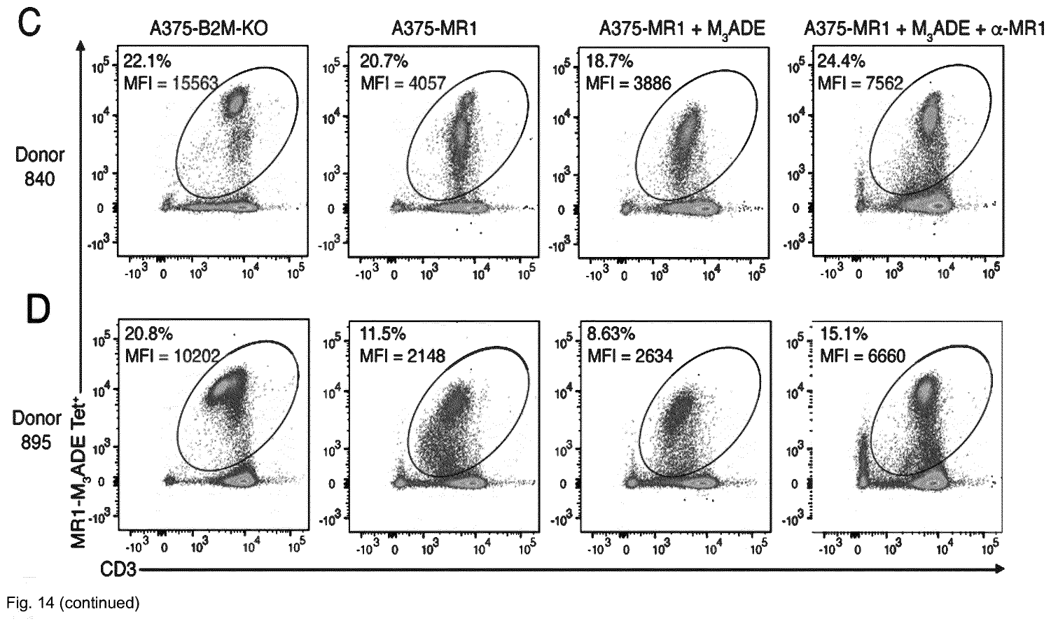

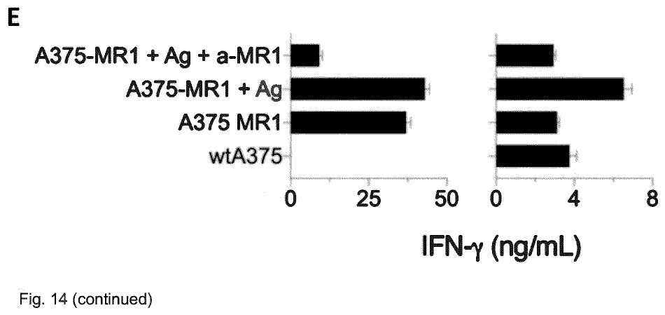

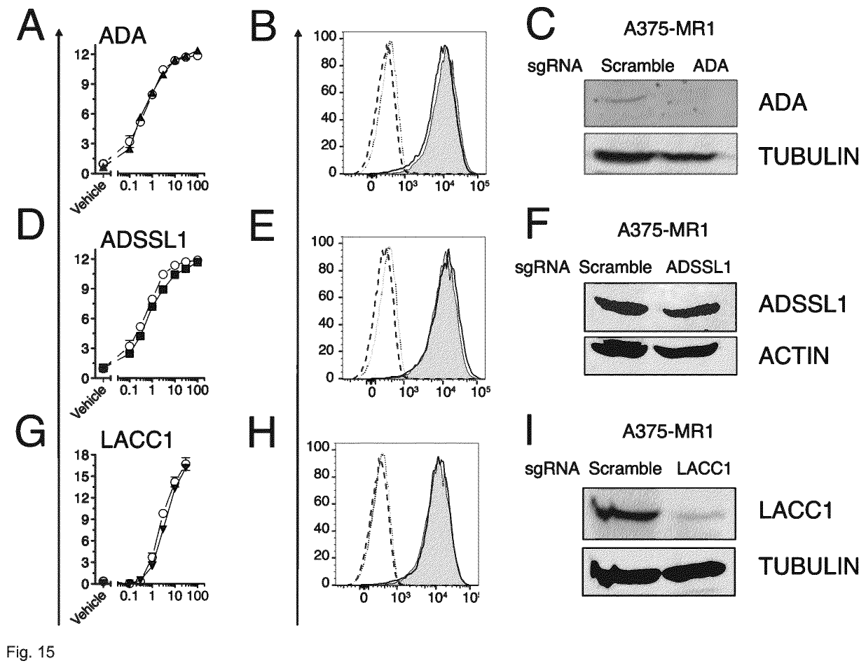

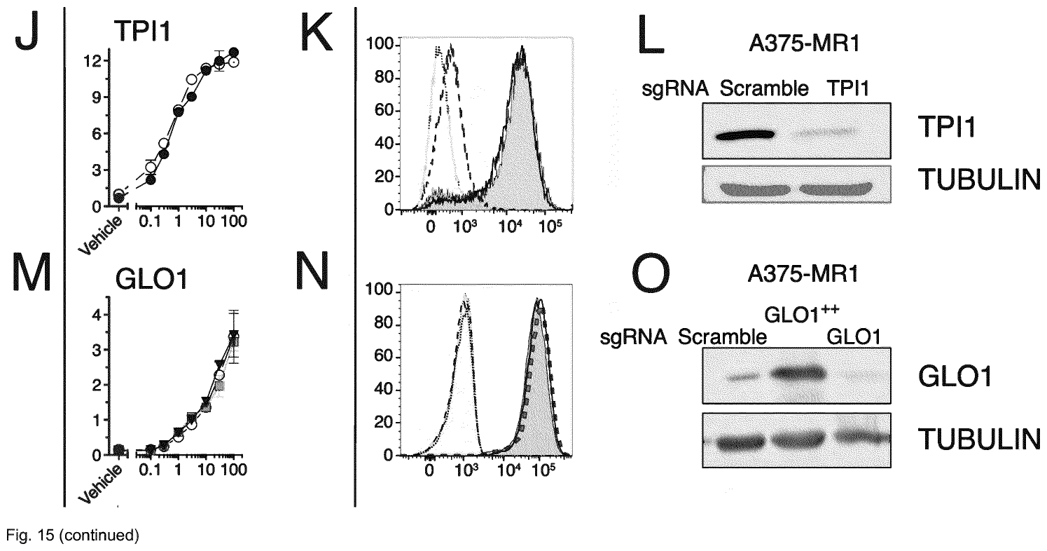

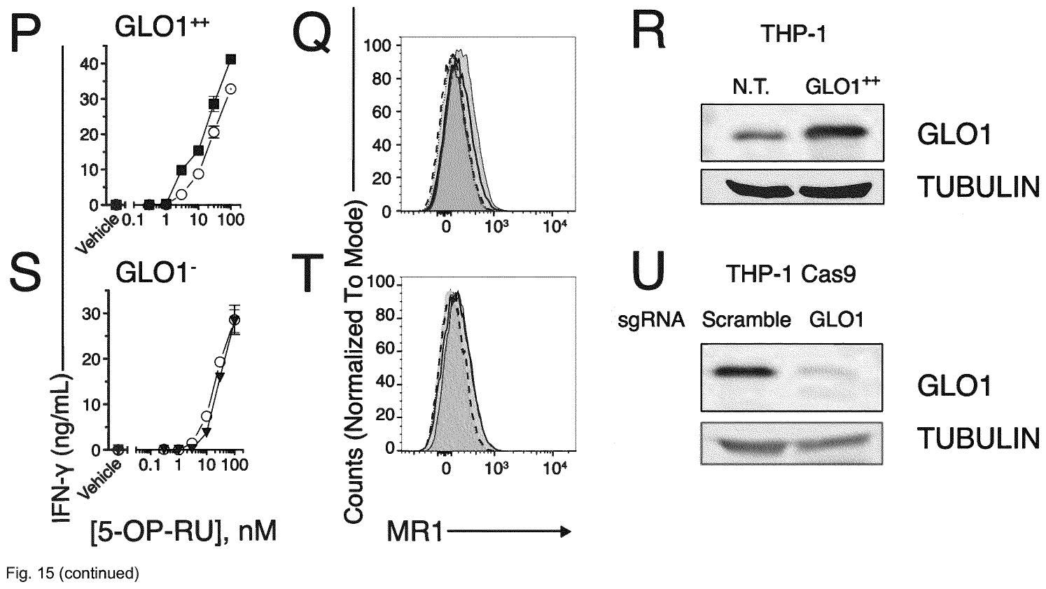

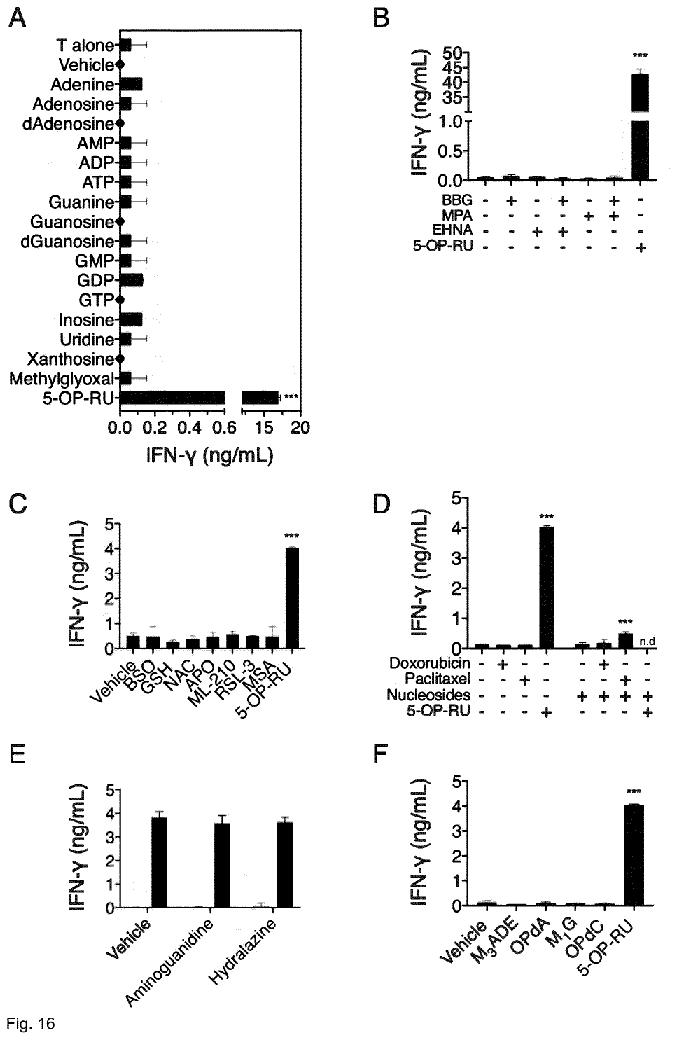

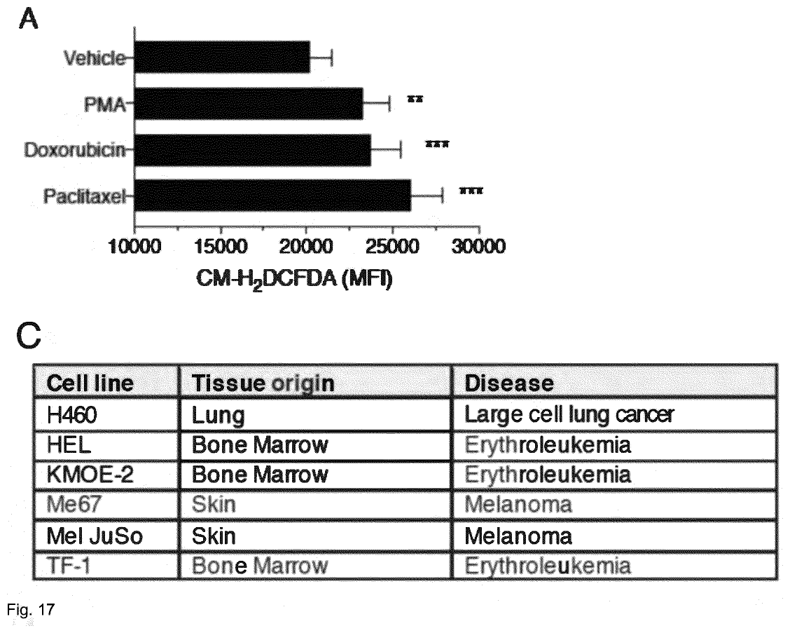

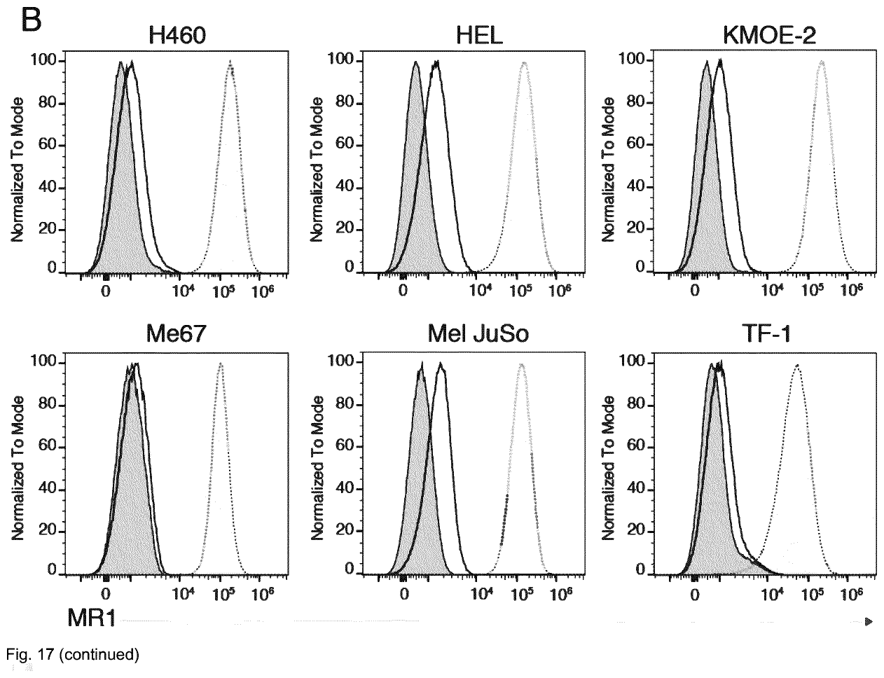

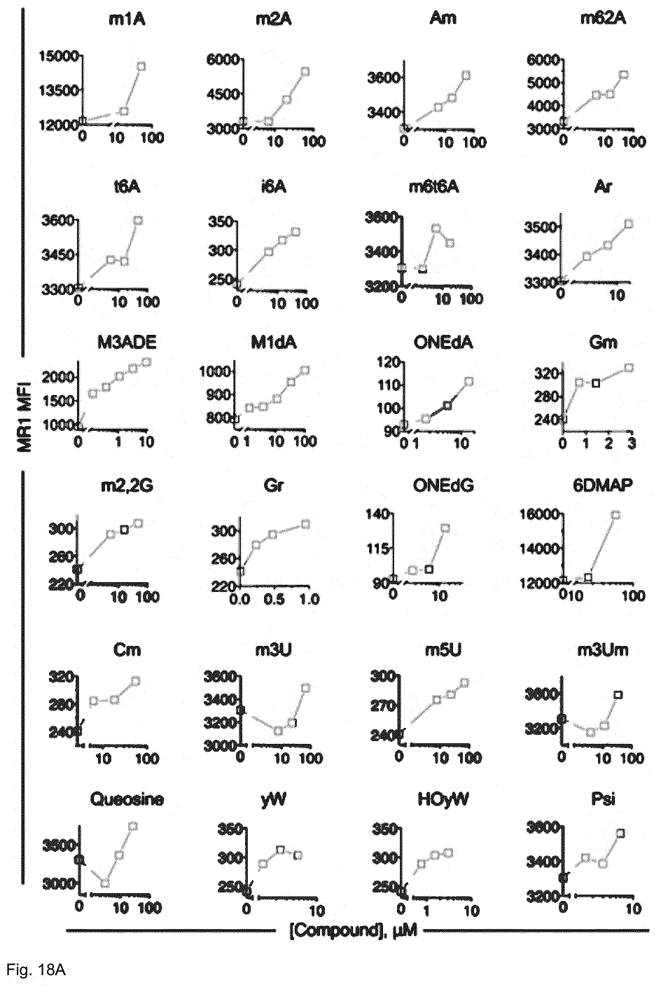

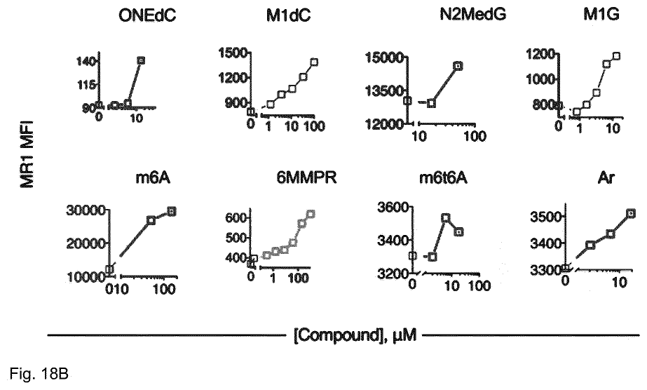

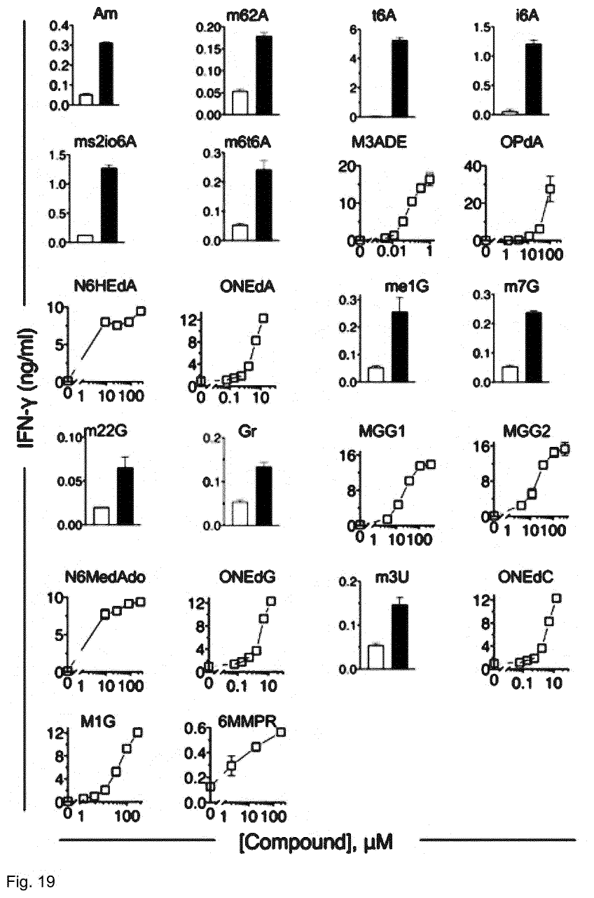

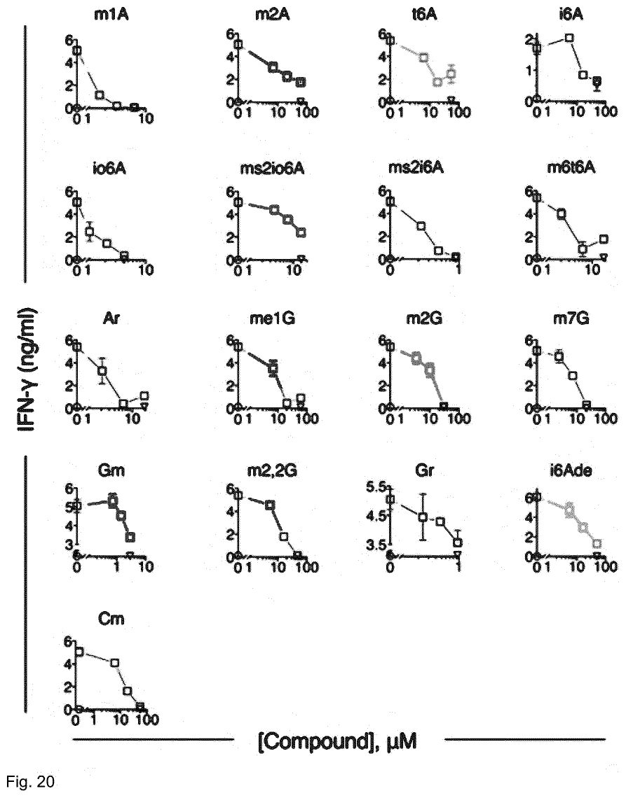

As proof of concept to demonstrate the use of these novel compounds for the detection and capture of MR1T cells, the inventors selected the compound M₃ADE. MR1 molecules containing the M₃ADE were generated by in vitro refolding of human recombinant soluble MR1 produced in bacteria. Properly refolded MR1 monomers loaded with M₃ADE were purified by gel filtration chromatography (FIG. 4a), and their capacity to stimulate MR1T cells, in the absence of APCs, was tested using plate-bound assays (FIG. 4b). The correctly refolded MR1-M₃ADE complexes were then biotinylated and tetramerized using streptavidin. MR1-M₃ADE tetramer specificity was validated through the ex vivo capture of reactive T cells from PBMCs, followed by their expansion and generation of T-cell clones which maintained the original characteristics of tetramer reactivity and the expected compound specificity. As example, one such clone, named AVA34, was confirmed by positive staining with MR1-M₃ADE tetramers but negative with MR1-5-OP-RU tetramers (FIG. 5a). Abrogation of MR1-M₃ADE tetramer binding by pre-incubation with anti-V3 antibodies (anti-Vβ8 mAbs), further confirmed TCR specificity of the tetramer staining (FIG. 5a). The clone AVA34 was also specifically activated upon re-exposure to M₃ADE but no other compounds presented by MR1⁺APCs (FIG. 5b). After these validation experiments, MR1-M₃ADE tetramers were used to detect reacting MR1T cells from freshly isolated PBMC of healthy donors (FIG. 6). MR1-M₃ADE tetramer positive cells were readily detected and their frequency ranged from −0.005% to 0.097% (mean 0.027%) of CD3⁺ cells, similar to conventional HLA-restricted T cells and to the frequencies previously reported for MR1T cells using different strategies. To promote the accumulation of carbonylated nucleosides, in some experiments, THP-1 cells were treated with drugs that increase the amounts of cellular carbonyls. These drugs (daidzin, disulfiram, oleanoic acid, ellagic acid) induced a strong T cell response in MR1T cell clones (FIG. 7, panels a, b, c, d, e). In further experiments THP-1 cells were treated with the adenosine deaminase inhibitor EHNA to induce accumulation of adenosine-containing adducts or with mycophenolic acid, which inhibits the enzyme inosine-5′-monophosphate dehydrogenase (IMPDH), thus inducing an increase of inosine and adenosine-containing nucleosides adducts. Both these drugs induced a strong stimulation of MR1T cells (FIG. 9, panel g and h).

### Single-Gene Knock-Outs in Metabolic Pathways Contribute to the Efficiency of T-Cell Mediated Killing

In previous studies the inventors isolated human T cells that recognize tumor cells expressing low levels of MR1 under sterile conditions. These MR1T cells, recognize tumor cells lines expanded in vitro or in vivo, indicating that stimulatory antigens preferentially accumulate in tumor cells, according to environmental conditions. Furthermore, individual MR1T cell clones showed patterns of tumor recognition suggesting the tumors bore shared and unique Ags that the inventors posited were of metabolic origin. Multiple approaches were used to identify these Ags.

In order to reveal key points within global metabolism contributing to the production of metabolite self-antigens, the inventors performed a genome-wide CRISPR knock-out screen.

An A375 melanoma tumor cell line that the inventors previously transfected with the MR1 and CAS9 genes (A375-MR1 cells), were used as a target for the cytotoxic MR1T cell clone TC5A87. After transduction with a library of sgRNAs covering the total human genome and three sequential killing rounds, surviving A375-MR1 cells were subjected to deep sequencing and enriched or depleted gRNAs were evaluated.

Prior to differential enrichment analysis, genes essential for growth of A375-MR1 cells were removed from the raw data to avoid confounding results with hits to gene-targets that are likely to be depleted in a non-T-cell dependent manner. It was also expected that many guides in the library would not affect the efficiency of T-cell mediated killing and therefore would display random enrichment and depletion. The GecKO v2 library contains non-targeting negative control guides, which should not display any significant enrichment or depletion in response to T-cell mediated killing, i.e. they should display random changes in abundance. Hence, a Log₂ fold-change cutoff based on the top and bottom 1% of Log₂ fold-changes in the negative control guides was used to identify guides that had been significantly enriched or depleted (FDR<0.05) more than background enrichment or depletion (i.e. random). The documented inequality of gRNA activity in the GeCKO library, hampers hit-selection using rank-based methodology, hence differential enrichment analysis was performed using the edgeR package. The inventors hypothesized knock-outs of genes involved in steps upstream of antigen biosynthesis will enhance the ability of A375-MR1 cells to escape T-cell mediated killing by reducing the generation of antigenic compounds. Conversely, knock-outs of genes involved in steps downstream of antigen biosynthesis will increase the accumulation of antigenic compounds and lead to increased T-cell mediated killing. Indeed, the results of differential enrichment analysis showed a fraction of enriched guides (n=243) that targeted 237 unique genes, including two-positive control genes, MR1 and beta-2-microglobulin (B2M) without which, antigens would not be presented to MR1-restricted T-cells. Among these genes the inventors also found enrichment of guides specific for adhesion molecules including CD58 (LFA-3), ICAM-1 that are the ligands of CD2 and CD11a on T cells, respectively. The latter interactions are important for T cell recognition of target cells and were also found in a previous CRISPR screening of tumor cell killing by immune cells.

Among the depleted hits, 5331 guides that targeted 4705 unique genes were significantly depleted relative to the control cells transduced with the gRNA library not subjected to TC5A87 killing. Since gene-essentiality is dependent on context and sensitive to many experimental parameters, it should be expected that not all essential genes were successfully removed using the Hart A375-MR1 essential gene-set. Furthermore, the number of genes required for tumor cell survival should far outweigh the number of genes that allow escape from T-cell mediated killing, thus, it is unsurprising that many more significantly depleted than enriched guides were observed. Binomial enrichment analysis of gene-ontology (GO) terms annotated to significant hits, revealed a large number of depleted gene targets sharing enriched GO terms in Metabolic Processes, suggesting that metabolic processes important for MR1T cell stimulation require coordinated activity of several genes. This was less evident in enriched gene targets. Furthermore, these significant hits showed enrichment in Nucleobase and Nucleic Acid Metabolic Processes, suggesting a potential role for these metabolic pathways in MR1T cell stimulation.

To explore the effect of the significantly enriched and depleted gene targets identified in the screen on global metabolism, the inventors used structural sensitivity analysis, which was recently extended from reaction-level to gene-level perturbations, to predict the metabolic network response to single-gene knock-outs of genes in Recon3D, a genome-scale model of human metabolism. The model was appropriately pre-processed.

Pearson correlations between the genome-wide reaction sensitivities of each modelled knock-out were then used to identify sets of 2 or more knock-outs that had similar (Pearson score>0.6) global effects on metabolic reactions. This analysis led to selection of 125 genes among the significantly enriched or depleted genes found in the CRISPR screening. When these selected genes were correlated to their corresponding KEGG pathways, it became apparent that Oxidative phosphorylation and Purine Metabolism can be perturbed by single-gene knockouts of a number of different gene targets. Additionally, all of these genes, except 2, were among the significantly depleted hits identified in the screen. Independently, binomial enrichment analysis of KEGG Pathways represented by the 4705 depleted gene targets in the CRISPR screen or by the 125 genes selected from the in silico metabolic model, identified that the Purine Metabolic Pathway is significantly enriched in both the CRISPR-screen hits as a whole and the subset of correlated Recon3D models (Binomial p-value=2.00e⁻³ and 1.67e⁻⁴. While not identified as a significantly enriched pathway in the hits from the CRISPR screen, both Oxidative phosphorylation and Glycerolipid pathways were significantly enriched in the metabolic perturbation model (Binomial p-value=6.58e⁻¹³ and 4.08e⁻⁴.

### Nucleobase and Nucleoside Antigens Presented by Tumor Cells can Stimulate MRIT Cells

The inventors focused first on the purine pathway. Here the inventors identified several genes that might be involved in MR1T-cell antigen accumulation: Adenosine Deaminase (ADA), Adenylosuccinate Synthase 1 (ADSSL1), Laccase Domain Containing 1 (LACC1), cGMP-specific 3′,5′-cyclic phosphodiesterase (PDE5A), Aldehyde Dehydrogenase 16 Family Member A1 (ALDH16A1) and Hypoxanthine Phosphoribosyltransferase 1 (HPRT1). ADA converts adenosine to inosine, and ADSSL1 is necessary for the de novo production of adenosine monophosphate (AMP) from inosine monophosphate (IMP); while LACC1 enables the purine nucleoside cycle, and PDE5A catalyzes the specific hydrolysis of cGMP to 5′-GMP. Lastly, ALDH16A1 and HPRT1 proteins form a complex that generates purine nucleotides through the purine salvage pathway. All together, these findings further support purines as molecules potentially involved in target recognition by MR1T cells.

To validate the relevance of these genes, the inventors generated knock-out lines, which were individually tested for their competence to stimulate MR1T cells. ADA- and LACC1-deficient cells induced an increased stimulation as compared to parental A375-MR1 cells as measured by IFN-γ release (FIGS. 8A, B and 8D, E), whereas ADSSL1-deficient cells induced a slight but significant decreased stimulation (FIGS. 8C and 8F). These experiments were conducted using two different MR1T cell clones, selected because in preliminary experiments they showed different tumor recognition patterns. In control experiments, all gene knock-out cell lines equally stimulated MR1-restricted MAIT cells in the presence of the 5-OP-RU antigen (FIG. 15), and showed similar expression of MR1 on the cell surface, indicating that the observed altered MR1T cell stimulation was not due to a general alteration in antigen-presentation capability. These findings pointed the inventor's attention to purines as potential antigens.

To investigate the possible role of purines in MR1T-cell stimulation, the inventors incubated human acute monocytic leukemia THP-1 cells with synthetic nucleotides, nucleosides or nucleobases before adding MR1T-cell clones and measuring IFN-γ production. THP-1 cells were selected as targets because they constitutively express low surface levels of MR1 and induce some spontaneous MR1T-cell stimulation, demonstrating their ability to appropriately process and present MR1T-cell antigens. A375-MR1 cells expressing very high surface levels of MR1 were also included as a positive control to stimulate MR1T-cell cytokine production. The inventors found that three distinct MR1T-cell clones reacted to different groups of compounds: TC5A87 did not significantly respond to tested compounds (FIG. 8G, left); DGB129 reacted to adenine, adenosine, deoxyadenosine (dAdo) and inosine (FIG. 8G, middle); and MCA3C3 was activated by ADP, guanine, guanosine, deoxyguanosine and xanthosine (FIG. 8G, right). THP-1 cells incubated with the synthetic compounds did not stimulate MAIT cells (FIG. 16A). Interestingly, despite the compounds were used at high concentration, their stimulatory effect was minimal compared to MR1T stimulation with A375-MR1, suggesting that these molecules might be intermediate precursors of antigens.

Taken together, these findings show that human MR1T cell clones can recognize nucleobase/nucleoside antigens processed by and presented on a cancer cell line, and that cross-reactivity might exist between different MR1T-cell clones.

Methylglyoxal and Purine Metabolism Pathways within Tumor Cells Cooperate for MR1T-Cell Stimulation

To understand which metabolic pathways might be involved in recognizing nucleobase/nucleoside antigens on tumor cells by MR1T cells the inventors began by interrogating the inventors whole-genome gene disruption screening data. The inventors observed that some of the significantly depleted sgRNAs were related to genes involved in glycolysis (TPI1) and methylglyoxal (MG) degradation, including Glyoxalase 1 (GLO1), and Glyoxalase Domain Containing 4 (GLOD4). TP/1 encodes a triosephosphate isomerase within the glycolytic pathway and is responsible for the enzymatic conversion of dihydroxyacetone phosphate (DHAP) into glyceraldehyde 3-phosphate (G3P)—a reaction that can otherwise occur spontaneously with the generation of MG (FIG. 9). Conversely, GLO1-deficient cells are impaired in MG (a highly reactive carbonyl) degradation, which therefore accumulates. As MG forms adducts with several nucleobases, these data suggest a potential involvement of MG in generating MR1T-cell antigens.

The inventors then dissected the possible roles of glycolysis and MG degradation in MR1T stimulation by generating single gene KO cell lines. Loss of TPI1 in A375-MR1 cells significantly increased IFN-γ production by both MR1T-cell clones (FIG. 9A, B). Furthermore, A375-MR1 cells pulsed with glucose and then fixed showed increased MR1T-cell stimulatory capacity (FIG. 9C, D); this effect was abolished when the same cells were incubated with deoxyglucose (FIG. 9C, D), which does not enter the glycolytic pathway and so does not generate MG. The inventors also saw that GLO1-deficient and GLO1-overexpressing A375-MR1 cells showed increased and reduced MR1T-cell stimulatory capacity, respectively (FIG. 9E, F). Altogether these results suggest that MG accumulation in target cells is important for the stimulation of MR1T-cell clones. To investigate the potential synergism between MG and purine metabolic pathways in MR1T cell stimulatory capacity of tumor cells, the inventors explored the effects of individual and combined pharmacologic inhibition of key enzymes in these pathways. The inventors used S-p-bromobenzylglutathione (BBG) to inhibit GLO1; mycophenolic acid (MPA) to inhibit inosine monophosphate dehydrogenases (IMPDH1, 2), leading to IMP accumulation; and erythro-9-(2-Hydroxy-3-nonyl) adenine hydrochloride (EHNA) to inhibit ADA and phosphodiesterase 2 (PDE2), inducing adenosine, dAdo and cGMP accumulation. To sensitively detect the effects of the inhibitors, the inventors again used THP-1 cells as target cells: the inventors found that BBG in combination with each of the other two drugs significantly enhanced the IFN-γ release of both MR1T cell clones to THP-1 cells (FIG. 9G, H). The DGB129 clone was more sensitive to these treatments and also reacted to THP-1 cells treated with EHNA, BBG, or MPA alone (FIG. 9H).

The inventors tested the IFN-γ response of MR1T cells to GLO1-modified THP-1 cells and various doses of MG or dAdo. The inventors found that MG treatment significantly increased MR1T-cell stimulation by GLO1-deficient compared to wild-type THP-1 cells (FIG. 9I). Conversely, MG failed to induce MR1T-cell stimulation when administered to GLO1-overexpressing cells (FIG. 9J). Similarly, MR1T reactivity to dAdo was increased using GLO1-deficient THP-1 cells as antigen-presenting cells (APCs) (FIG. 9K) and decreased using GLO1-overexpressing THP-1 cells (FIG. 9L). Together, these findings suggest that nucleosides/nucleobases and MG cooperate in generating potential MR1T-cell antigens.

Multiple Oxidative Stress-Related Carbonyl Species Accumulate within Tumor Cells and Contribute to MR1T Cell Stimulation

In addition to the purine pathway, the inventor's model-based analysis highlighted genes related to oxidative phosphorylation, whose protein products participate in ATP generation within mitochondria and whose alteration promotes accumulation of reactive oxygen species (ROS). Alongside, the analysis pointed towards the relevance of the H⁺ transporter subunits ATP6V1C2, TCIRG1 and ATP6V0D2 involved in coupling proton transport and ATP hydrolysis and thus contributing to maintaining the organelle physiological milieu in the cell, including mitochondria. Therefore, the inventors next investigated the roles of ROS in MR1T-cell stimulation by tumor cells.

First, the inventors focused on genes involved in oxidative phosphorylation. The inventors initial MR1T-cell killing screen uncovered a significant depletion of sgRNAs specific for GSTM1, GSTA4, GSTA1, GSTM5, GSTA2, GSTA3, GSTM3, and GSTO1; these genes are involved in the detoxification of electrophilic compounds and ROS by their conjugation to glutathione (GSH), a ROS scavenger. The inventors therefore hypothesized that in the absence of GSTs, and upon accumulation of ROS and electrophilic molecules, tumour cells may accumulate MR1T-cell stimulatory compounds. Accordingly, the inventors tested the effects of paclitaxel and doxorubicin, two drugs that induce cellular accumulation of O₂⁻ and H₂O₂. Both drugs significantly increased ROS accumulation (FIG. 17A) and promoted activation of all three MR1T-cell clones when incubated with THP-1 cells and nucleoside compounds (FIG. 10A-C), mirroring the additive effects observed when purine-modifying drugs and carbonyl-degradation inhibitors were combined (FIG. 9G, H).

Next, the inventors treated A375-MR1 with apocynin, an O₂⁻ scavenger and NADPH oxidase inhibitor, or with GSH or N-acetylcysteine (NAC), which prevent H₂O₂ accumulation before fixation and incubation with the three different MR1T cell clones. The inventors found that A375-MR1 cells treated with any of the inhibitors stimulated significantly less IFN-γ production from MR1T cells, with apocynin being effective with one T-cell clone (FIG. 10D-F). The inventors also treated A375-MR1 cells with buthionine sulfoximine (BSO), an inhibitor of GSH synthase, and a significant increase was observed in the stimulation of all the tested MR1T clones (FIG. 10D-F). Together, these data show that ROS participate in MR1T antigen accumulation, although requires concomitant alteration of nucleobases metabolism.

Peroxide accumulates in many tumor types and is involved in various signal transduction pathways and cell fate decisions. Peroxide is also necessary for lipid peroxidation, a pathway that generates malondialdehyde (MDA) and 4-OH-nonenal (4-HNE), two highly reactive carbonyls. Both compounds form stable adducts with proteins, lipids and nucleobases and accumulate within tumor cells. Alongside the inventors findings that inhibiting ROS accumulation impedes tumor cell stimulation of MR1T cells (FIG. 10D-F), the inventors inferred a role for lipid peroxidation from the results of the inventors CRISPR/Cas9 screen, which showed significant depletion of the glutathione peroxidase 4 (GPX4) and glutathione peroxidase 1 (GPX1) sgRNAs. While GPX1 protein catalyzes the reduction of organic hydroperoxides and H₂O₂ by glutathione, GPX4 has a high preference for lipid hydroperoxides and protects cells against membrane lipid peroxidation and death. Accordingly, when the inventors pre-treated A375-MR1 cells with the selective GPX1 inhibitor mercaptosuccinic acid (MSA), or with two GPX4 inhibitors RSL3 and ML-210, they showed significantly increased MR1T-cell stimulatory activity (FIG. 10G-1). None of these compounds influenced the MAIT-cell response to microbial antigens in control experiments, except paclitaxel that in presence of nucleosides could induce a little but significant stimulation (FIG. 16C-D). Taken together, these findings indicate that peroxides and lipid peroxidation contribute to MR1T-cell stimulation by tumor cells.

To further assess the carbonyl involvement in the generation of MR1T antigens, the inventors tested the capacity of carbonyl scavengers to prevent MR1T-cell activation. A375-MR1 cells were incubated with aminoguanidine and hydralazine, which show preferential scavenging activity for different carbonyls, then fixed and washed before the addition of MR1T cell clones. The inventors found that both scavengers showed significant inhibition of IFN-γ production by MR1T cell clones (FIG. 10J-L) and had no effect on MAIT cell activation (FIG. 16E).

Collectively, the inventor's data suggest that multiple oxidative stress-related reactive carbonyl species accumulating in cells following metabolic alterations combine with nucleobases to generate MR1-presented antigens that stimulate MR1T cells.

### Evaluation of the Biological Activity of Compounds

Cell surface MR1 modulation was measured on THP-1 MR1 cells (10⁵ cells/well) after incubation for 3 h at 37° C. in the presence or absence of compounds (3 doses each). Ac-6-FP (100 μM, Schircks Laboratories Cat #11.418) was used as positive control compound for MR1 surface upregulation. MR1 expression was evaluated by staining with mouse mAbs anti-human-MR1 APC-labeled (IgG2a,k clone 26.5, Biolegend Cat #361108) and subtracting the background staining with APC-labeled mouse IgG2a,k isotype control antibodies (Biolegend Cat #400220), which was always below 300 MF.

Competition assays were performed by incubating APCs (10⁵ cells/well) with compounds (3 doses each) for 2 h at 37° C., then adding the antigen for each clone of interest at optimal concentration (≥EC₅₀) and incubating for 2 additional h before the addition of T cells (10⁴ cells/well). As positive control for competition with antigens, Ac-6-FP was used (100 μM). Supernatants were collected after 24 h for cytokine analysis measured by ELISA.

### Nucleobase Adducts Stimulate MR1T Cells

The biochemical condensation of carbonyl species with nucleobases is a unique feature of many cancer cell types, and leads to the generation of adducts. The inventors considered the possibility that nucleobase-adduct-containing compounds might be MR1T-cell antigens. To test this hypothesis, the inventors synthesized four previously described adducts: the purine adducts 8-(9H-purin-6-yl)-2-oxa-8-azabicyclo[3.3.1]nona-3,6-diene-4,6-dicarbaldehyde (M₃ADE), N5-(3-oxo-1-propenyl)-2′-deoxyadenosine (OPdA), and pyrimido[1,2-a]purin-10(3H)-one (M₁G), and the pyrimidine adduct, N⁴-(3-oxo-1-propenyl)-2′-deoxycytidine (OPdC), and investigated their antigenic activity (FIG. 11). The inventors confirmed the identities of the adducts by high-resolution electro-spray ionization mass spectrometry (HR-ESI-MS) and nuclear magnetic resonance (NMR) spectroscopy.

Initial experiments showed that these compounds were capable of inducing MR1 upregulation on pulsed APC, with the exception of MGG (FIG. 11).

T cell activation experiments using APC expressing low levels of MR1 showed that each compound was stimulatory for different MR1T cells (FIG. 11). Dose-response studies revealed different degrees of sensitivity of individual MR1T cells. In some instances, extremely low EC₅₀ (1-10 nM) were observed, whereas the same antigens were weakly stimulatory for other MR1T clones or not stimulatory at all. A second important finding was the antigen cross-reactivity of some MR1T clones. However, these clones showed preferential response to one tested antigen, in line with the type of antigen cross-reactivity described with T cells specific for peptide or lipid antigens presented by MHC or CD1 molecules, respectively. These data are in agreement with the cross-reactivity observed in the experiments of FIG. 8G. THP-1 cells pulsed with a range of doses of the adducts showed that individual compounds differentially stimulated IFN-γ production by distinct MR1T-cell clones and showed cross-reactivity of some clones toward multiple compounds (FIG. 11). In all cases, MR1T-cell activation by adduct-loaded THP-1 cells was fully inhibited by the addition of blocking anti-MR1 monoclonal antibodies (mAbs), confirming MR1 restriction of adduct recognition (FIG. 11).

When the inventors incubated THP-1 cells with the adducts the inventors found that they all induced a 1.5-5 fold increase in the average expression level of MR1 on the cell, as found with other MR1-binding compounds (FIG. 11A-E). THP-1 cells pulsed with a range of doses of the adducts showed that individual compounds differentially stimulated IFN-γ production by distinct MR1T-cell clones and showed cross-reactivity of some clones toward multiple compounds (FIG. 11A-E). In all cases, MR1T-cell activation by adduct-loaded THP-1 cells was fully inhibited by the addition of blocking anti-MR1 monoclonal antibodies (mAbs), confirming MR1 restriction of adduct recognition (FIG. 11A-E).

Next, the inventors sought to investigate whether these synthetic antigens were stimulatory without modifications inside APC. Plate-bound recombinant MR1 protein loaded with synthetic antigens efficiently stimulated specific MR1T clones, thus excluding a requirement for intracellular processing (FIG. 12A). Thus, carbonyl-nucleobase adducts directly bind MR1 without further modifications and stimulate MR1T-cells. All these stimulations were fully inhibited by addition of blocking anti-MR1 mAbs confirming the role of MR1 protein.

The inventors further confirmed the stimulatory capacity of these antigens by extending the activation assays to include additional MR1T-cell clones expressing different TCRs: of the fourteen randomly selected MR1T clones tested, eight reacted to at least one ligand. M₃ADE significantly stimulated six clones, OPdA eight clones, M₁G and OPdC three clones each (FIG. 12B). Again, distinct patterns of adduct recognition resulted in a wide range of IFN-γ production levels by MR1T cells, alongside MR1T-clonal cross-reactivity. The inventors also found that M₁G or M₃ADE when added to clones QY1A16 and QY1C₃, respectively, resulted in significant decreased response to THP-1 cells, suggesting that both adducts compete with endogenous stimulatory molecules in THP-1 cells. These findings also suggested that some MR1T clones recognize antigens different from the tested ones. Of note, THP-1 cells pulsed with the synthetic compounds did not stimulate the MAIT-cell clone MRC25 (FIG. 16F).

The inventors then used plastic-bound recombinant MR1 molecules loaded with synthetic antigens to assess whether THP-1 cell processing of the adducts was needed for MR1T-cell recognition: the inventors found that it was not (FIG. 12A). Thus, carbonyl-nucleobase adducts directly bind MR1 without further modifications and stimulate MR1T-cells.

Next, the inventors asked how MR1T clones recognizing different synthetic compounds differ in their capacity to react to different tumor cell lines expressing physiological low levels of MR1. This question is relevant as preferential accumulation of unique carbonyl adducts in each tumor cell line may result in preferential stimulation of individual MR1T cells. The clone AVA34 activated by M₃ADE, reacted specifically to KMOE-2 and HEL tumor cell lines; the clone QY1A16 that is stimulated by OPdA and OPdC selectively reacted to H460, Juso and KMOE-2 tumor cell lines; the clone AC1A4 that is activated by M₃ADE, OPdA and M₁G reacted to all six tested tumor cell lines, and the clone TC5A87 that is activated by OPdA, MGG, and OPdC reacted to all tumor cell lines (FIG. 12C). Importantly, all reactivities were inhibited by addition of anti-MR1 mAbs, thus demonstrating the MR1 restriction of tumor recognition (FIG. 12C). It is also of note that the tested tumor lines derived from different tissues. The tested MR1T clones did not react to leukocytes from healthy individuals, with the exception of AVA34 and TC5A87 clones that released very low, albeit significant, quantities of IFN-γ upon incubation with monocytes (FIG. 12C).

In conclusion, MR1T cells recognize compounds containing intact carbonyl adducts of nucleobases presented on MR1 molecules. Some MR1T clones seem to be specific for one or another adduct; others show a degree of cross-reactivity. The broad recognition and the broad distribution of nucleobase adducts in many cancers may justify the broad reactivity of MR1T cells to tumors derived from different tissues.

MR1 Tetramers Loaded with Nucleobase Adduct-Containing Metabolites Detect MR1T Cells

To identify and characterize ex vivo MR1T cells reactive to nucleobase adduct-containing metabolites, the inventors generated MR1 tetramers loaded with the synthesized adducts. The inventors focused on M₃ADE, as it was the most efficient in increasing MR1 surface expression, showed the highest potency, and was recognized by 6/14 tested MR1T-cell clones. The inventors performed several experiments to confirm proper protein refolding and MR1-M₃ADE tetramer-specific MR1T-cell staining. MR1-M₃ADE monomers stimulated clone DGB129 in plate-bound assays (FIG. 4A-B), and fluorescently-labeled tetramerized MR1-M₃ADE monomers stained the MR1T-cell clone AC1A4 (FIG. 5A), consistent with its M₃ADE reactivity (FIG. 12A); whereas the tetramers failed to bind a canonical MAIT clone (MRC25; FIG. 13A).

For further validation, the MR1-M₃ADE tetramers were used to isolate MR1T cells from peripheral blood mononuclear cells (PBMCs) and establish a novel series of clones. These clones were able to bind MR1-M₃ADE tetramers but not MR1-5-OP-RU or MR1-6-FP tetramers, demonstrated by the representative clone AVA34 (FIG. 13B). Abrogation of binding using an anti-TCRVβ monoclonal antibody, further confirmed TCR specificity of the tetramer staining (FIG. 13C). In addition, the MAIT clone MRC25 was labeled with the MR1-5-OP-RU tetramer but not with the MR1-M₃ADE tetramer (FIG. 5). Importantly, clone AVA34 reacted to THP-1 cells loaded with M₃ADE, while failed to respond to other tested antigens (FIG. 13C). Thus, these experiments validated the capacity of the MR1- M₃ADE tetramer to bind M₃ADE-specific T cells.

The inventors next asked whether MR1-M₃ADE tetramers could bind and identify specific MR1T cells ex vivo in the blood of healthy donors. Screening of peripheral blood mononuclear cells (PBMCs) from nine healthy individuals revealed tetramer-positive cells in all individuals with the frequencies ranging between 0.006% and 0.077% (median=0.01%) of total CD3⁺ cells (FIG. 13D, E and FIG. 6).

Alongside the marked inter-individual differences in the frequency of MR1-M₃ADE tetramer-binding T cells, the in inventors also uncovered notable phenotypic variations across the tetramer positive populations. While most cells expressed CD8α (range 38%-97%), one donor displayed a distinct CD4⁺ cell population that accounted for 57% of all tetramer-positive cells (FIG. 13F). In another donor, the CD4⁻/CD8⁻ population accounted for 33% of tetramer-positive cells (FIG. 13F). According to CD45RA and CCR7 expression, the inventors also observed large donor-specific differences in the frequency of tetramer-positive naïve cells (range 8%-55%, median 29%), central memory (range 7%-48%, median 22%), effector memory (range 5%-81%, median 23%), and TEMRA (range<1%-42%, median 3.8%) (FIG. 13G, H).

Thus, MR1T cells that recognize carbonyl-nucleoside-adducts are present in the blood of healthy individuals; they display a heterogeneous phenotype, and potentially undergo phenotypic differentiation in a donor-specific manner.

### MR1T Cells Reactive to Nucleobase Adduct-Containing Metabolites Infiltrate Tumor Tissue

As all tested individuals possessed potentially M₃ADE-reactive T cells in their blood, the inventors next asked whether M₃ADE-reactive T cells could be detected in tumor samples. The inventors isolated TILs from non-small cell-lung-cancer biopsies from two patients and co-cultured them with A375-MR1 cells loaded with M₃ADE, in order to expand MR1T cells. After expansion, the inventors were able to detect MR1-M₃ADE tetramer-binding cells in TIL co-cultures from both patients (FIG. 14A, B). In donor #840, these cells were CD4⁺ (52.6%) or CD8⁺ (42%); whereas in donor #895 they were largely CD8⁺ (93.5%) (FIG. 14A, B). To confirm their antigen specificity, the inventors enriched by sorting MR1-M₃ADE tetramer-positive cells and stimulated them again with either A375-p2mKO cells, A375-MR1 or A375-MR1 loaded with M₃ADE. The inventors measured the extent of MR1T-cell activation via TCR downregulation, which occurred only in the presence of A375-MR1 cells and M₃ADE-loaded A375-MR1 cells, and was prevented by anti-MR1 blocking mAbs (FIG. 14C, donor 840, and FIG. 14D, donor 895). The inventors further confirmed MR1-dependent activation of tetramer-positive T cells by measuring IFN-γ release (FIG. 14E). Cells from donor #840 responded to A375-MR1 cells irrespective of the presence of M₃ADE (FIG. 14C, E), whereas cells from donor 895 reacted only to M₃ADE-loaded A375-MR1 cells (FIG. 14D, E); in both cases the response was inhibited by anti-MR1 mAbs (FIG. 14 C-E). This data confirms the presence of M₃ADE-reactive T cells within the tumor environment, thus indicating a potential role for MR1T cells in tumor immunity.

### Discussion

In this study, the inventors have identified nucleobase adduct-containing metabolites as self-antigens capable of stimulating human T lymphocytes recognizing MR1-expressing tumor cells. Previous studies showed that MR1T cells react to unique compounds fractionated from tumor cells, suggesting distinct antigen specificity. Here, the inventors confirm those data and extend them to show that structurally-diverse nucleobase adduct-containing compounds bind MR1 and stimulate individual MR1T cells. Both purines and pyrimidines form antigenic adducts and different carbonyls participate in their generation, confirming that MR1 is a molecule with versatile antigen-binding capacity.

Carbonyls accumulate as a consequence of different metabolic alterations in glycolysis and lipid peroxidation, during the metabolism of biogenic amines, vitamins and steroids, as well as upon biotransformation of environmental agents and drugs. How many carbonyls are involved warrants further investigation: this is an important question as the number and diversity of carbonyl species participating in the generation of MR1-presented nucleobase adducts may determine the size and variety of the MR1T cell antigen repertoire. It is of note that some MR1T clones did not react to any of the antigens tested here, whereas still responded to A375-MR1 cells, suggesting the MR1T cell antigen repertoire can be quite heterogeneous.

While carbonyl accumulation is important, it is not sufficient to stimulate MR1T cells, as concomitant availability of free purines and pyrimidines must occur within the target cells. The structures of modified nucleobases are suited to MR1 binding and resemble those of other MR1 ligands, as they are composed of differently modified heterocyclic compounds.

Important players in the generation of immunogenic nucleobase adducts are ROS—side products of oxidative phosphorylation that promote tumor induction and tumor cell proliferation. ROS also promote lipid peroxidation leading to carbonyl generation, thus also exerting indirect effects on accumulation of MR1T cell antigens. Indeed, treatment with ROS-inducing drugs enhanced MR1T-target cell stimulatory capacity, which in turn was dampened by the addition of ROS scavengers. Thus, MR1T-cell antigens are derived by combined alterations of multiple metabolic pathways, leading to the accumulation of nucleobases, carbonyls and ROS.

An important unanswered question in the field has been the basis of the specificity of tumor cell recognition by MR1T cells. The inventors' data provide a plausible answer to this, as most normal cells at steady state physiologically regulate the distinct metabolic pathways contributing to nucleobase adduct generation and accumulation. By contrast, tumor cells frequently become altered in many of these pathways sustaining cell proliferation, including alterations in glucose and glutamine uptake, and high cellular demand for reduced nitrogen. Indeed, many tumors increase the transcription of key genes involved in the de novo purine synthesis, suggesting a key role for purinosomes. Furthermore, tumor cells are prone to DNA damage through the generation of nucleic acid adducts formed upon DNA oxidation and interaction with end products of lipid peroxidation.

Importantly, in normal cells, several mechanisms are involved in scavenging highly reactive carbonyls, through which carbonyls are oxidized to carbonic acids, are conjugated with glutathione, or are reduced to less toxic alcohols. In the inventors' CRISPR/Cas9 screen, the inventors identified several genes involved in these processes that were conserved, suggesting that they contributed to escaping MR1T-cell recognition. The relevance of these controlling mechanisms is further supported by the negative effect of carbonyl scavengers on MR1T-stimulatory capacity of tumor cells.

MR1T-cell recognition of nucleobase adduct-containing metabolites raises the question of the physiological role of these cells. It is conceivable to attribute them a potential role in surveying cells abnormally accumulating compounds responsible for DNA alterations and therefore predisposed to dangerous genetic mutations. The ubiquitous expression of MR1 might be instrumental to this function of cellular metabolic integrity control. Together these properties of MR1T cells make them attractive targets for immunotherapeutic use in cancer. The inventors envisage the possibility of using selected MR1T TCR genes to redirect the specificity of cancer patient T cells toward these novel tumor-associated metabolite antigens, and therefore equip them with tumor-targeting capacity. The detection of MR1T cells within the tumor microenvironment in two lung cancer patients is promising evidence that supports the potential value of this strategy. Another possible application is the use of nucleobase adduct-containing metabolites as components of innovative anti-tumor vaccines. Importantly, the monomorphic nature of MR1 might offer the possibility to circumvent HLA-polymorphism and design T-cell-based immunotherapies applicable to the entire population of cancer patients on universal basis and independent of genetic background.

In conclusion, the immune system continues to surprise us with its capacity to detect a wide repertoire of structurally-variable antigens. T-cell recognition of nucleobase adduct-containing metabolites is the most recent evidence of this enormous flexibility.

### REFERENCES

Geacintov, N. E. and S. Broyde (2010). The chemical biology of DNA damage. Weinheim, Wiley-VCH.
Ishiwata et al. (1995). “Comparison of serum and urinary levels of modified nucleoside, 1-methyladenosine, in cancer patients using a monoclonal antibody-based inhibition ELISA.” Tohoku J Exp Med 176(1): 61-68.
Kawai, Y. and E. Nuka (2018). “Abundance of DNA adducts of 4-oxo-2-alkenals, lipid peroxidation-derived highly reactive genotoxins.” J Clin Biochem Nutr 62(1): 3-10.
Kim, C. S., S. Park and J. Kim (2017). “The role of glycation in the pathogenesis of aging and its prevention through herbal products and physical exercise.” J Exerc Nutrition Biochem 21(3): 55-61.
Marnett, L. J. (2002). “Oxy radicals, lipid peroxidation and DNA damage.” Toxicology 181-182: 219-222.
Richarme et al. (2017). “Guanine glycation repair by DJ-1/Park7 and its bacterial homologs.” Science 357(6347): 208-211.
Riggins et al. (2004). “Kinetic and thermodynamic analysis of the hydrolytic ring-opening of the malondialdehyde-deoxyguanosine adduct, 3-(2′-deoxy-beta-D-erythro-pentofuranosyl)-pyrimido[1,2-alpha]purin-10(3H)-one.” J Am Chem Soc 126(26): 8237-8243.
Seidel, A., S. Brunner, et al. (2006). “Modified nucleosides: an accurate tumor marker for clinical diagnosis of cancer, early detection and therapy control.” Br J Cancer 94(11): 1726-1733.
Stone et al. (1990). “Investigation of the Adducts Formed by Reaction of Malondialdehyde with Adenosine” Chem. Res. Toxicol. 3: 33-38.
Voulgaridou et al. (2011). “DNA damage induced by endogenous aldehydes: current state of knowledge.” Mutat Res 711(1-2): 13-27.
Wauchope et al. (2015). “Nuclear Oxidation of a Major Peroxidation DNA Adduct, M₁dG, in the Genome.” Chem Res Toxicol 28(12): 2334-2342.
Lepore et al. (2017). Functionally diverse human T cells recognize non-microbial antigens presented by MR1. ELife 6: e24476.
Lepore et al. (2014). Parallel T-cell cloning and deep sequencing of human MAIT cells reveal stable oligoclonal TCRbeta repertoire. Nat Commun 5, 3866.
Dai et al. (2014). edgeR: a versatile tool for the analysis of shRNA-seq and CRISPR-Cas9 genetic screens. F1000 Research 3, 95.
Hart et al. (2015). High-Resolution CRISPR Screens Reveal Fitness Genes and Genotype-Specific Cancer Liabilities. Cell 163, 1515-1526.
Brunk et al. (2018). Recon3D enables a three-dimensional view of gene variation in human metabolism. Nature Biotechnology 36, 272-281.
Kanehis et al. (2019). New approach for understanding genome variations in KEGG. Nucleic Acids Res 47, D590-D595.
Schmaler et al. (2018). Modulation of bacterial metabolism by the microenvironment controls MAIT cell stimulation. Mucosal Immunology 11: 1060-1070.
Langmead, B., and Salzberg, S. L. (2012). Fast gapped-read alignment with Bowtie 2. Nat Methods 9, 357-359.
Sanson et al. (2018). Optimized libraries for CRISPR-Cas9 genetic screens with multiple modalities. Nat Commun 9, 5416.
Bolger, A. M., Lohse, M., and Usadel, B. (2014). Trimmomatic: a flexible trimmer for Illumina sequence data. Bioinformatics 30, 2114-2120.
Durinck et al. (2009). Mapping identifiers for the integration of genomic datasets with the R/Bioconductor package biomaRt. Nature protocols 4, 1184-1191.

TABLE 2
Summary of compound functional activity determined to date

| | | Functional assay | | | |
|---|---|---|---|---|---|
| Cmpd | | MRI | MRI | | Reactive T cells |
| n° | Cmpd short name | upregulation | downmodulation | Competition | isolated |
| 1 | m1A | YES | NO | YES | NO |
| 2 | m2A | YES | NO | YES | NO |
| 3 | Am | YES | NO | NO | YES |
| 4 | m6,6A or m6,2A | YES | NO | NO | YES |
| 5 | t6A | YES | NO | YES | YES |
| 6 | t6A | YES | NO | YES | YES |
| 7 | io6A | NO | NO | YES | NO |
| 8 | ms2io6A | YES | NO | NO | YES |
| 9 | ms2i6A | NO | NO | YES | NO |
| 10 | m6t6A | YES | NO | YES | YES |
| 11 | Ar(p) | YES | NO | YES | NO |
| 12 | M1dA or OPdA | YES | NO | ND | YES |
| 13 | M3ADE | YES | NO | ND | YES |
| 14 | ONEdA | YES | NO | NO | ND |
| 15 | me1G | NO | NO | YES | YES |
| 16 | m2G | YES | NO | NO | NO |
| 17 | m7G | NO | NO | YES | YES |
| 18 | Gm | YES | NO | YES | NO |
| 19 | m2,2G | YES | NO | YES | YES |
| 20 | Gr | YES | NO | YES | YES |
| 21 | MGG (6M) | NO | NO | ND | YES |
| 22 | MGG (7M) | NO | NO | ND | YES |
| 24 | M1G | YES | NO | ND | YES |
| 25 | ONEdG | YES | NO | NO | ND |
| 29 | Cm | YES | NO | YES | NO |
| 30 | m3U | YES | NO | YES | YES |
| 31 | m5U | YES | NO | YES | NO |
| 32 | m3Um | YES | NO | YES | NO |
| 33 | Q | YES | NO | YES | NO |
| 34 | yW | YES | NO | YES | NO |
| 35 | OHyW | YES | NO | YES | NO |
| 36 | Psi | YES | NO | YES | NO |
| 37 | ONEdC | YES | NO | NO | ND |
| 38 | M1dC or OPdC | YES | NO | ND | NO |
| 40 | 6-MMPr | YES | NO | NO | YES |
| 42 | m6A | YES | NO | YES | NO |
| 43 | MeP | NO | YES | YES | NO |
| 44 | 6DMAP | YES | NO | YES | NO |
| 45 | i6Ade | NO | YES | YES | NO |
| 47 | MeG | NO | YES | NO | NO |
| 48 | N2MedG | YES | NO | NO | NO |
| 52 | MTA | NO | NO | YES | NO |
| 53 | N6MedAdo | NO | NO | ND | YES |
| 54 | N6HEdA | NO | NO | ND | YES |

ND, not determined

TABLE 3A
Designation of sequence ID Nos of MR1
specific TCRs of PCT/EP2019/074284

| | | Protein | | | | | |
|---|---|---|---|---|---|---|---|
| Clone | Clone | | | α | β | Nucleic acid | |
| No | Name | α | β | CDR3 | CDR3 | α | β |
| 1-o | MCA2E7 | 1 | 2 | 65 | 80 | 7 | 8 |
| 2-o | MCA3C3 | 3 | 4 | 66 | 81 | 9 | 10 |
| 3-o | CHO9A4 | 5 | 6 | 67 | 82 | 11 | 12 |
| 4-o | DGA4 | 13 | 25 | 68 | 83 | 37 | 49 |
| 5-o | DGA28 | 14 | 26 | 69 | 84 | 38 | 50 |
| 6-o | DGB70 | 15 | 27 | 70 | 85 | 39 | 51 |
| 7-o | DGB129 | 16 | 28 | 71 | 86 | 40 | 52 |
| 8-o | CH9A3 | 17 | 29 | 72 | 87 | 41 | 53 |
| 9-o | JM64-8 | 18 | 30 | 73 | 88 | 42 | 54 |
| 10-o | JMA | 19 | 31 | 74 | 89 | 43 | 55 |
| 11-o | LMB1F3 | 20 | 32 | 75 | 90 | 44 | 56 |
| 12-o | MCA3D9 | 21 | 33 | 76 | 91 | 45 | 57 |
| 13-o | TC5A87 | 22 | 34 | 77 | 92 | 46 | 58 |
| 14-o | SMC3 | 23 | 35 | 78 | 93 | 47 | 59 |
| 15-o | TRA44 | 24 | 36 | 79 | 94 | 48 | 60 |
| Clone | Clone | | | γ | δ | | |
| No | Name | γ | δ | CDR3 | CDR3 | γ | δ |
| 1-gdo | MGDA1G5 | 61 | 62 | 95 | 96 | 63 | 64 |

TABLE 3B
Designation of sequence ID Nos of new MR1
specific TCRs first disclosed herein

| | | Protein | | | | | |
|---|---|---|---|---|---|---|---|
| Clone | Clone | | | α | β | Nucleic acid | |
| No | Name | α | β | CDR3 | CDR3 | α | β |
| 1-N | AC1A4 | 97 | 98 | 99 | 100 | 133 | 134 |
| 2-N | AC1B76 | 101 | 102 | 103 | 104 | 135 | 136 |
| 3-N | AVA34 | 105 | 106 | 107 | 108 | 137 | 138 |
| 4-N | AVA46 | 109 | 110 | 111 | 112 | 139 | 140 |
| 5-N | LMC1D1 | 113 | 114 | 115 | 116 | 141 | 142 |
| 6-N | MCA2B1 | 117 | 118 | 119 | 120 | 143 | 144 |
| 7-N | QY1A16 | 121 | 122 | 123 | 124 | 145 | 146 |
| 8-N | QY1B42 | 125 | 126 | 127 | 128 | 147 | 148 |
| 9-N | QY1C3 | 129 | 130 | 131 | 132 | 149 | 150 |

TABLE 4
gRNA target sequences and cloning vectors.

| Target | | SEQ | |
|---|---|---|---|
| gene | gRNA sequence | ID N | Vector |
| ADA | CAGGCTTGATGGA | 151 | pLV-mCherry-U6 gRNA |
| | TCCGTCT | | |
| ADA | TCACCGTACTGTC | 152 | lentiGuide-Puro |
| | CACGCCG | | |
| ADA | GCGGTACAGTCCG | 153 | lentiGuide-Puro |
| | CACCTGC | | |
| ADSSL1 | TTCCAGGGGGGCA | 154 | lentiGuide-Puro |
| | ACAACGC | | |
| ADSSL1 | GCTGATGATGTCG | 155 | lentiGuide-Puro |
| | GCGTCCG | | |
| ADSSL1 | ACATACCGAAGTC | 156 | lentiGuide-Puro |
| | AATGTCG | | |
| LACC1 | CGTAGGTTGGCGA | 157 | lentiGuide-Puro |
| | ATGCTGC | | |
| LACC1 | TACCTTGGGATCT | 158 | lentiGuide-Puro |
| | CTCCGTT | | |
| LACC1 | TCAAGAAAATCTG | 159 | lentiGuide-Puro |
| | CGTAGGT | | |
| GLO1 | GAACCGCAGCCCC | 160 | pRP[gRNA]-EGFP: |
| | CGTCCGG | | P2A:Puro-U6 |
| GLO1 | GTCCGGCGGCCTC | 161 | pRP[gRNA]-EGFP: |
| | ACGGACG | | P2A:Puro-U6 |
| TPI1 | CGGCGAGGGCTTA | 162 | lentiGuide-Puro |
| | CCGGTGT | | |
| TPI1 | ACCGGTGTCGGCC | 163 | lentiGuide-Puro |
| | GGCACCT | | |
| TPI1 | CGAAGTCGATATA | 164 | lentiGuide-Puro |
| | GGCAGTA | | |
| Scrambled | GTGTAGTTCGACC | 165 | lentiGuide-Puro |
| sequence | ATTCGTG | | |
| Scrambled | GTTCAGGATCACG | 166 | lentiGuide-Puro |
| sequence | TTACCGC | | |
| Scrambled | AAATGTGAGATCA | 167 | lentiGuide-Puro |
| sequence | GAGTAAT | | |

1. A method for modulating an interaction between an MR1 polypeptide and an MR1-specific T cell receptor molecule, said method comprising contacting said MR1 polypeptide with
a. An MR1 ligand compound described by any one of the following general formulas: 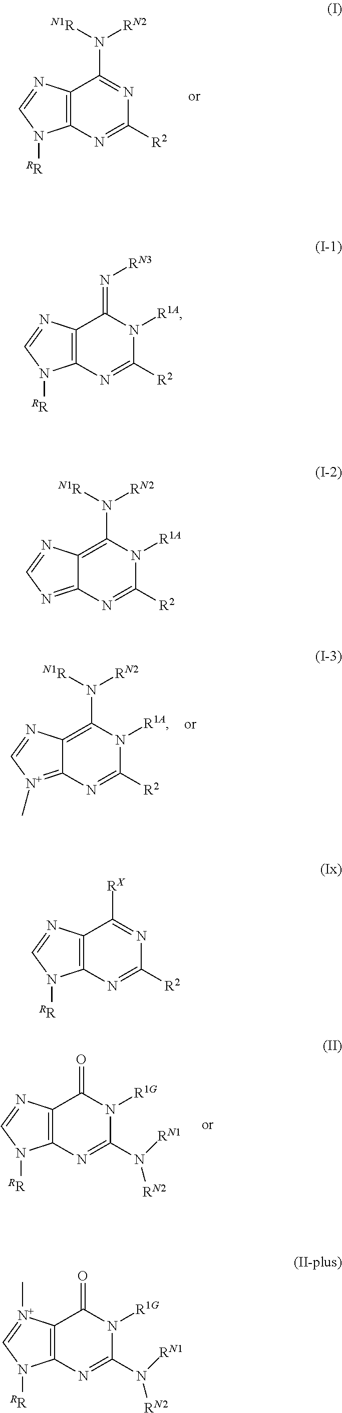 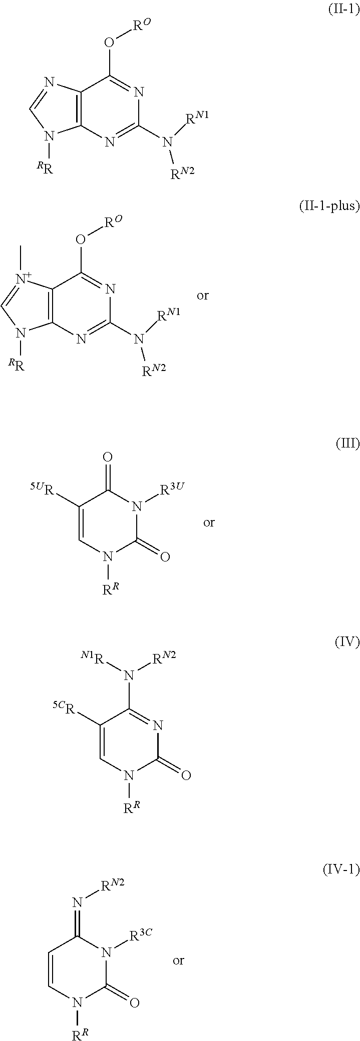
wherein
R^{1A} is H or methyl, R^{1G} is H or methyl;
R^{N3} is H or methyl;
R² is selected from H, methyl and —S-methyl;
R^{3U} and R^{5U} are selected from H and methyl; R^{5C} is selected from H and methyl;
R^{N1} and R^{N2} are both H or C₁ to C₃ alkyl,
or R^{N1} is H or C₁ to C alkyl (particularly R^{N1} is H or methyl) and R^{N2} is selected from a C₁-C₆ alkyl and a C₂-C₆ alkylene and a C₁-C₆ alkyl substituted carbamoyl, wherein the alkyl or alkylene is unsubstituted or substituted with carbonyl, carboxyl and/or hydroxyl,
particularly R^{N1} is H and R^{N2} is selected from a methyl, 2-hydroxy-ethyl, 1-carboxethyl, 1,2-dicarboxy-ethyl, threonylcarbamoyl, isopent-2-enyl, cis-hydroxyisopent-2-enyl, 3-oxo-1-propenyl, and hexa-1,3,5-triene-1,1,3-tricarbaldehyde;
or R^{N1}, R^{N2} and the nitrogen together form a 2-oxa-8-azabicyclo [3.3.1] nona-3,6-diene-4,6-dicarbaldehyde bi-annular system;
or
R^{N3} and R^{1A} together, or R^{N2} and R^{3C} together, or R^{N1} and R^{1G} together form an unsubstituted or C₄ to C₁₆-2-oxo-alkyl-substituted or □-carboxy-2-oxo-alkyl-substituted imidazole ring, or
R^{N3} and R^{1A} together, or R^{N2} and R^{3C} together, or R^{N1} and R^{1G} together are —C(CH₃)OH—CHOH— or C(R′)OH— CH₂—CHOH— or oxy-cyclopropylidene-malonaldehyde-substituted prop-2-ene with R′ being selected from H, CH₃, CH(OH)C₂H₅, C₂H₅ and C₄H₉;
or R^{N1} and R^{1G} form a pyrimidine, or R^{N1} and R^{1G} or R^{N3} and R^{1A} form a 12-oxo-5,6,10,12-tetrahydro-3H-6,10-methano[1,3,5]oxadiazocine ring system, or R^{N1} and R^{1G} form a 2-oxa-6,8-diazabicyclo [3.3.1] nona-3-ene-4-carbaldehyde bi-annular system; and
R^{O} is selected from H, unsubstituted or hydroxyl-substituted C₁-C₅ alkyl or C₂-C₅ alkylene,
R^{X} is selected from SH, C₁-C₅ alkyl, C₂-C₅ alkylene, and C₁-C₅ S-alkyl;
R^{R} is selected from H, 1′-ribosyl, 2′-deoxy-1′-ribosyl, 5′-phospho-1′-ribosyl, 5′-methylthio-1′-ribosyl, 1′-(2′-O-ribosyl-5″-phosphate)ribosyl, 1′-(2′-O-ribosyl)-ribosyl 1′-(2′-O-methyl)ribosyl;
with the proviso that adenine, adenosine, deoxyadenosine, guanine, guanosine, deoxyguanosine, uracil, uridine, deoxyuridine, thymine, thymidine, deoxythymidine (5-methyluridine), cytosine and cytidine and deoxycytidine are not encompassed by the general formulas I, II, III and IV,
b. or with an MR1 ligand compound selected from 3-methyladenine (41), 7-methyl-7-deaza-2′-deoxyguanosine (49), queuosine (33), wybutosine (34), hydroxywybutosine (35) or pseudouridine (36), or with (2R,3S,4R,5R)-2-(hydroxymethyl)-5-(6-(methylthio)-9H-purin-9-yl)tetrahydrofuran-3,4-diol (40).

2. The method according to claim 1, wherein R^{R} is described by the general formula (V) or (V-S) 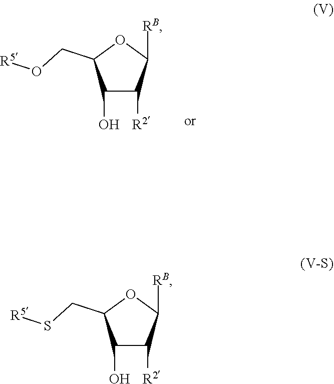
wherein R^{B} is the bond connecting the moiety to the N⁹ nitrogen of I, I-1, II, II-plus, II-1, II-1-plus, Ix, or to the N¹ nitrogen of III, IV or IV-1, R^{2′} is selected from H, OH, OCH₃, O-ribosyl and O-ribosyl-5″-phosphate, and R^{5′} is selected from H and PO₃²⁻;
particularly wherein R^{R} is described by the general formula (V),
more particularly wherein R^{R} is described by the general formula (Va) 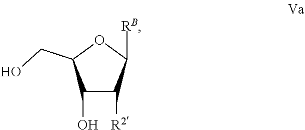
even more particularly wherein R^{R} is described by the general formula (Vb: R^{2′} is OH) or (Vc: R^{2′} is 5′-phosphoribosyl) or (Vd: R^{2′} is H) or (Ve: R² is ribosyl):

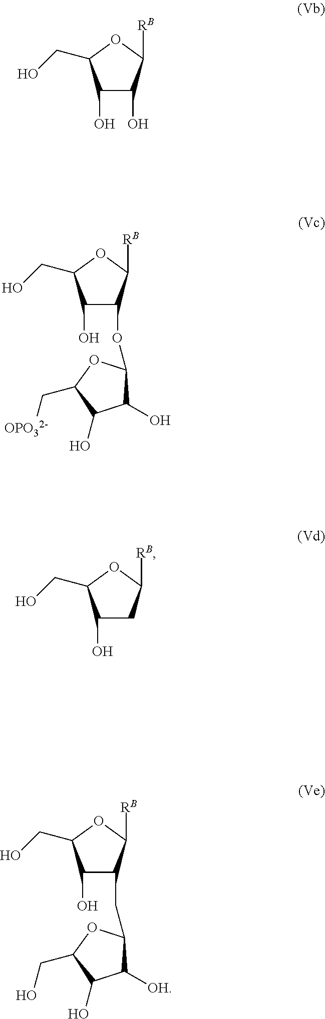

3. The method according to claim 1, wherein the MR1 ligand compound is described by I-1 or the general formula IIa 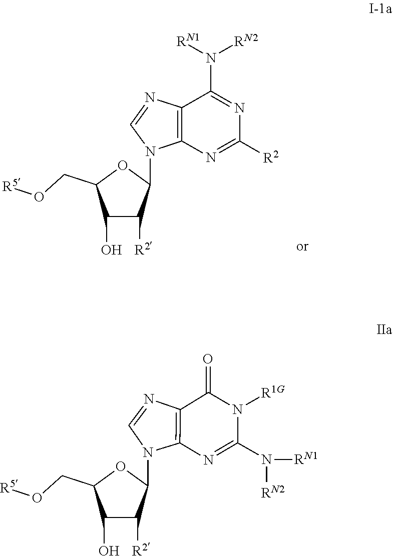
wherein R^{N1}, R^{R}, R^{N2}, R^{1A}, R^{1G}, R^{2′} and R^{5′} can have the meaning indicated above.

4. The method according to claim 1, wherein the MR1 ligand compound is described by formula I and R^{N1}, R^{N2} and the nitrogen together form a 2-oxa-8-azabicyclo [3.3.1] nona-3,6-diene-4,6-dicarbaldehyde bi-annular system.

5. The method according to claim 1, wherein the MR1 ligand compound is described by
a. formula (I) wherein R² is S-methyl and R^{N1} and R^{N2} are both H;
b. formula (I) wherein R² is methyl and R^{N1} and R^{N2} are both H; or
c. formula (I-1) wherein R^{1A} is methyl, R² is H and R^{N3} is H; or
d. formula (I-1) wherein R^{1A} is methyl, R² is methyl and R^{N3} is H; or
e. formula (I-1) wherein R^{1A} is methyl, R² is S-methyl and R^{N3} is H; or
f. formula (I) wherein R² is H, one of R^{N1} and R^{N2} is selected from H and methyl, and the other one of R^{N1} and R^{N2} is selected from methyl, 3-methylbut(2)enyl, 3-hydroxymethylbut(2)enyl and threonylcarbamoyl; or
g. formula (I) wherein R² is S-methyl and one of R^{N1} and R^{N2} is selected from H and methyl, and the other one of R^{N1} and R^{N2} is selected from methyl, ethan-2-ol, 1,2-dicarboxy-ethyl, 3-methylbut(2)enyl, 3-hydroxymethylbut(2)enyl and threonylcarbamoyl;
h. formula (I) wherein R² is methyl and one of R^{N1} and R^{N2} is selected from H and methyl, and the other one of R^{N1} and R^{N2} is selected from methyl, 3-methylbut(2)enyl, 3-hydroxymethylbut(2)enyl and threonylcarbamoyl;
i. formula (II) wherein R^{1G} is methyl, and R^{N1} and R^{N2} are both H; or
j. formula (II-plus) wherein R^{1G} is methyl, and R^{N1} and R^{N2} are both H; or
k. formula (II-plus) wherein R^{1G} is H, and R^{N1} and R^{N2} are both H; or
l. formula (II) wherein R^{1G} is methyl, and one of R^{N1} and R^{N2} is selected from H and methyl, and the other one of R^{N1} and R^{N2} is selected from methyl, ethyl, 3-methylbut(2)enyl, 3-hydroxymethylbut(2)enyl and threonylcarbamoyl; or
m. formula (II-plus) wherein R^{1G} is methyl, and one of R^{N1} and R^{N2} is selected from H and methyl, and the other one of R^{N1} and R^{N2} is selected from methyl, 3-methylbut(2)enyl, 3-hydroxymethylbut(2)enyl and threonylcarbamoyl; or
n. formula (II-plus) wherein R^{1G} is H, and one of R^{N1} and R^{N2} is selected from H and methyl, and the other one of R^{N1} and R^{N2} is selected from methyl, 3-methylbut(2)enyl, 3-hydroxymethylbut(2)enyl and threonylcarbamoyl; or
o. formulas (I-IM or II-IM), with R^{IM} selected from H, CH₂COCₙH₍₂ₙ₊₁₎ with n from 3 to 7 (particularly n=5), and CH₂CO(CH₂)ₘCOO⁻ with m from 3 to 9 (particularly n=7) 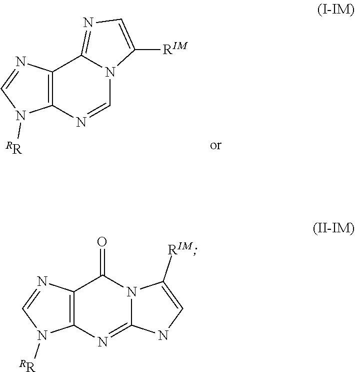
or
p. formulas (II-c), (II-d), (II-e) (II-f) 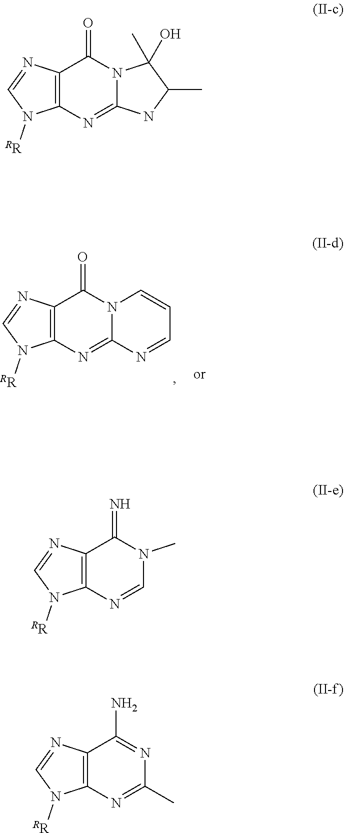
q. formulas (II-g) or (II-h), wherein R² is selected from H, methyl and S-methyl: 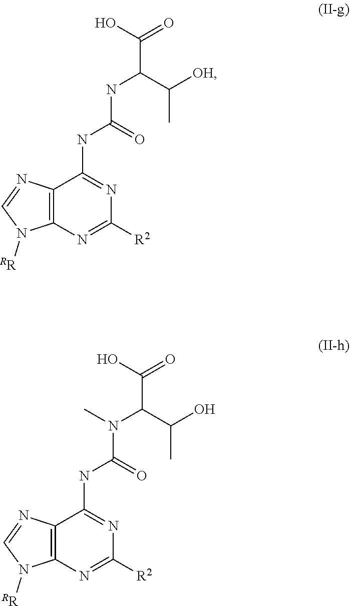
r. formulas (II-i) (II-j), wherein R^{N1} is selected from H and methyl and wherein R² is selected from H, methyl and S-methyl: 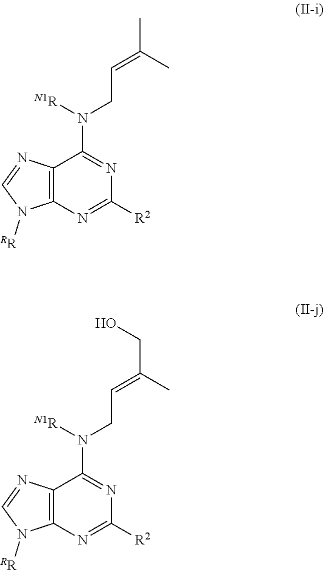
s. formula (III), wherein R^{3U} is H and R^{5U} is methyl,
t. formula (III), wherein R^{3U} is methyl and R^{5U} is H,
u. formula (III), wherein R^{3U} and R^{5U} are both methyl,
v. formula (IV), wherein R^{5C} is H, one of R^{N1} and R^{N2} is selected from H and methyl, and the other one of R^{N1} and R^{N2} is selected from methyl, 3-methylbut(2)enyl, 3-hydroxymethylbut(2)enyl and threonylcarbamoyl;
w. formula (IV-1), wherein R^{N2} is selected from methyl, 3-methylbut(2)enyl, 3-hydroxymethylbut(2)enyl and threonylcarbamoyl; or
x. formula (IV), wherein R^{5C} is methyl, one of R^{N1} and R^{N2} is selected from H and methyl, and the other one of R^{N1} and R^{N2} is selected from methyl, 3-methylbut(2)enyl, 3-hydroxymethylbut(2)enyl and threonylcarbamoyl;
y. formula (Ix), wherein R² is H, and R^{X} is selected from methyl and S-methyl;
z. formula (I), wherein R² is H, R^{N1} is H, R^{N2} is 3-oxo-1-propenyl;
aa. formula (I-a), 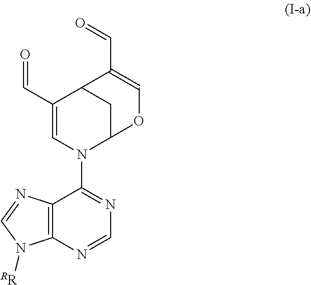
bb. formula (II-k), 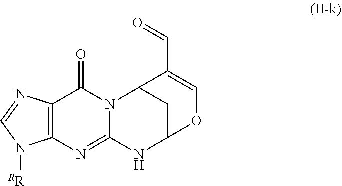
cc. formula (I-2), wherein R^{1A}, R^{N1} and R² are H, and R^{N2} is isopent-2-enyl, or cis-hydroxyisopent-2-enyl,
dd. formula (I), wherein R^{N1} and R² are both H, and R^{N2} is isopent-2-enyl, or cis-hydroxyisopent-2-enyl,
ee. formula (II-1), wherein R^{O} is methyl or ethan-2-ol, and R^{N1} and R² are both H;
ff. formula (I-b),

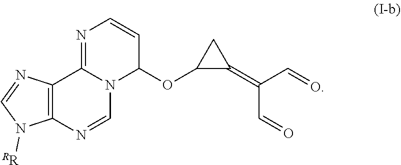

6. (canceled)

7. (canceled)

8. (canceled)

9. The method according to claim 1, wherein the MR1 ligand compound is selected from
a. 1-methyladenosine (1)
b. 2-methyladenosine (2)
c. 2′-O-methyladenosine (3)
d. N6,N6-dimethyladenosine (4)
e. N6-threonylcarbamoyladenosine (5)
f. N6-isopent-2-enyladenosine (6)
g. N6-(cis-hydroxyisopent-2-enyl) adenosine (7)
h. 2-methylthio-N6-(cis-hydroxyisopent-2-enyl) adenosine (8)
i. 2-methylthio-N6-isopent-2-enyladenosine (9)
j. N6-methyl-N6-threonylcarbamoyladenosine (10)
k. 2′-O-ribosyladenosinephosphate (11)
l. N6-(3-oxo-1-propenyl)-2′-deoxyadenosine (12)
m. 8-(9H-purin-6-yl)-2-oxa-8-azabicyclo[3.3.1]nona-3,6-diene-4,6-dicarbaldehyde (13)
n. 1-(3-((2R,4S,5R)-4-hydroxy-5-(hydroxymethyl) tetrahydrofuran-2-yl)-3H-imidazo[2,1-r] purin-7-yl) heptan-2-one (14)
o. 1-methylguanosine (15)
p. N2-methylguanosine (16)
q. 7-methylguanosine (17)
r. 2′-O-methylguanosine (18)
s. N2,N2-dimethylguanosine (19)
t. 2′-O-ribosylguanosine (20)
u. 3-((2R,3R,4S,5R)-3,4-dihydroxy-5-(hydroxymethyl)tetrahydrofuran-2-yl)-6,7-dihydroxy-6-methyl-6,7-dihydro-3H-imidazo[1,2-a]purin-9(5H-one (21), 3-((2R,3R,4S,5R)-3,4-dihydroxy-5-(hydroxymethyl)tetrahydrofuran-2-yl)-6,7-dihydroxy-7-methyl-6,7-dihydro-3H-imidazo[1,2-a]purin-9(5H)-one (22), or a mixture of the two;
v. 2-((6-oxo-6,7-dihydro-1H-purin-2-yl)amino)propanoate (23)
w. 3-((2R,5R)-4-hydroxy-5-(hydroxymethyl) tetrahydrofuran-2-yl)-7-(2-oxoheptyl)-3H-imidazo[1,2-a]purin-9(5H)-one (25)
x. 3-(2-deoxy-β-D-erythro-pentofuranosyl)pyrimido[1,2-a]purin-10(3H)-one (26)
y. N2-oxopropenyl-deoxyguanosine (27)
z. 3-((2R,4S,5R)-4-hydroxy-5-(hydroxymethyl)tetrahydrofuran-2-yl)-12-oxo-5,6,10,12-tetrahydro-3H-6,10-methano[1,3,5]oxadiazocino[5,4-a]purine-9-carbaldehyde (28)
aa. 2′-O-methylcytidine (29)
bb. 3-methyluridine (30)
cc. 5-methyluridine (31)
dd. 3,2′-O-dimethyluridine (32)
ee. 6-((2R,4S,5R)-4-hydroxy-5-(hydroxymethyl)tetrahydrofuran-2-yl)-3-(2-oxoheptyl)-1,8a-dihydroimidazo[1,2-c]pyrimidin-5(6H)-one (37)
ff. N4-(3-oxo-1-propenyl)-2′-deoxycytidine (38)
gg. 8-(1-((2R,4S,5R)-4-hydroxy-5-(hydroxymethyl)tetrahydrofuran-2-yl)-2-oxo-1,2-dihydropyrimidin-4-yl)-2-oxa-8-azabicyclo[3.3.1]nona-3,6-diene-4,6 dicarbaldehyde (39)
hh. 3-methyladenine (41)
ii. N6-methyladenosine (42)
jj. 6-methylpurine (43)
kk. 6-(dimethylamino)purine (44)
ll. N6-(A2-isopentenyl) adenine (45)
mm. N1-methyl-2′-deoxyguanosine (46)
nn. 1-methylguanine (47)
oo. N2-methyl-2′-deoxyguanosine (48)
pp. 7-methyl-7-deaza-2′-deoxyguanosine (49)
qq. 06-methyl-2′-deoxyguanosine (50)
rr. N2-ethyl-2′-deoxyguanosine (51)
ss. 5′-deoxy-5′-(methylthio)adenosine (52)
tt. N6-methyl-2′-deoxyadenosine (53)
uu. N6-(2-hydroxyethyl)-2′-deoxyadenosine (54)
vv. 06-(2-hydroxyethyl)-2′-deoxyguanosine (55)
ww. N6-succinyl adenosine (56)
xx. 2-(2-((3-((2R,4S,5R)-4-hydroxy-5-(hydroxymethyl)tetrahydrofuran-2-yl)-3,7-dihydropyrimido[2,1-,]purin-7-yl)oxy)cyclopropylidene)malonaldehyde (57);
yy. pyrimido[1,2-a]purin-10(3H)-one (M₁G) (24).

10. The method according to claim 1, wherein the method is selected from
a. a method for the identification and isolation of T cells or T cell receptor molecules reactive to the MR1 ligand compound when the MR1 ligand compound is presented by an MR1 molecule, and
b. a method for the identification and isolation of an antibody reactive to the MR1 ligand compound when the MR1 ligand compound is presented by an MR1 molecule, and
c. a method of diagnosis, wherein a sample obtained from a patient is analyzed with regard to the presence of an MR1 ligand compound as identified herein, particularly wherein the MR1 ligand compound is presented by or associated with an MR1 molecule on a patient's cell, more particularly wherein the MR1 ligand compound is presented by or associated with an MR1 molecule on a patient's cancer cell
d. a method of diagnosis, wherein a sample obtained from a patient is analyzed with regard to the presence of a T cell reactive towards an MR1 ligand compound as identified herein, wherein the MR1 ligand compound is presented by or associated with an MR1 molecule on a cell, particularly a patient's cell, more particularly wherein the MR1 ligand compound is presented by or associated with an MR1 molecule on a patient's cancer cell.

11. An MR1 ligand compound as specified in claim 1 for use in prophylaxis or treatment of a disease associated with an aberrant or absent MR1-specific T cell response, particularly in treatment of cancer characterized by tumor cells expressing MR1.

12. (canceled)

13. The MR1 ligand compound for use according to claim 11, wherein the compound is administered in association with (administered prior to, concomitant with or after administration of) a preparation comprising (transgenic) MR1-reactive T cells and/or a polynucleotide expression vector encoding MR1.

14. (canceled)

15. A method for identification of a T cell reactive to a MR1 ligand compound as specified in claim 1, said method comprising the steps
providing a preparation of T cells reactive to/capable of specifically recognizing MR1;
a. contacting said preparation of T cells with a complex comprising isolated MR1 associated to said compound;
b. isolating a T cell that is specifically reactive to said MR1 ligand compound in an isolation step.

16. An isolated T cell receptor (TCR), particularly a TCR comprising
an α chain of TCR and a β chain of a TCR,
or a TCR comprising a γ chain of a TCR and a δ chain of a TCR, particularly an α chain of TCR and a β chain of a TCR;
wherein the TCR is capable to specifically bind to an MR1 ligand compound as specified in claim 1 in association to an MR1 polypeptide, with the proviso that the TCR formed by association of SEQ ID NO 1 and 2, 3 and 4, 5 and 6, 13 and 25, 14 and 26, 15 and 27, 16 and 28, 17 and 29, 18 and 30, 19 and 31, 20 and 32, 21 and 33, 22 and 34, 23 and 35, 24 and 36, and 61 and 62 are disclaimed; and wherein the TCR recognizes an MR1 ligand compound in association with MR1, and wherein the MR1 ligand compound is selected from:
a. 1-methyladenosine (1);
b. 2-methyladenosine (2);
c. 2′-O-methyladenosine (3), with the proviso that the TCR composed of SEQ ID NO 22 and 34 is disclaimed;
d. N⁶, N⁶-dimethyladenosine (4), with the proviso that the TCRs composed of SEQ ID NO 1 and 2, and of 22 and 34 are disclaimed;
e. N⁶-threonylcarbamoyladenosine (5), with the proviso that the TCR composed of SEQ ID NO 22 and 34 is disclaimed;
f. N⁶-isopentenyladenosine (6), with the proviso that the TCRs composed of SEQ ID NO 1 and 2, 16 and 28, and of 22 and 34 are disclaimed;
q. N6-(cis-hydroxyisopentenyl) adenosine (7);
h. 2-methylthio-N⁶-(cis-hydroxyisopentenyl) adenosine (8), with the proviso that the TCRs composed of SEQ ID NO 1 and 2, 16 and 28, and of 22 and 34 are disclaimed;
i. 2-methylthio-N6-isopentenyladenosine (9);
i. N6-methyl-N⁶-threonylcarbamoyladenosine (10), with the proviso that the TCR composed of SEQ ID NO 22 and 34 is disclaimed;
k. 2′-O-ribosyladenosine (phosphate) (11);
l. NV-(3-Oxo-1-propenyl)-2′-deoxyadenosine (12), with the proviso that the TCRs composed of SEQ ID NO 1 and 2, 16 and 28, and of 22 and 34 are disclaimed;
m. 8-(9H-purin-6-yl)-2-oxa-8-azabicyclo[3.3.1]nona-3,6-diene-4,6-dicarbaldehyde (13), with the proviso that the TCR composed of SEQ ID NO 16 and 28 is disclaimed;
n. 1-(3-((2R,4S,5R)-4-hydroxy-5-(hydroxymethyl) tetrahydrofuran-2-yl)-3H-imidazo[2,1-i]purin-7-yl) heptan-2-one (14);
o. N2-methylguanosine (16);
p. 7-methylguanosine (17), with the proviso that the TCR composed of SEQ ID NO 22 and 34 is disclaimed;
q. 2′-O-methylguanosine (18);
r. N2, N2-dimethylguanosine (19), with the proviso that the TCR composed of SEQ ID NO 1 and 2 is disclaimed;
s. 2′-O-ribosylguanosine phosphate (20), with the proviso that the TCR composed of SEQ ID NO 22 and 34 is disclaimed;
t. 3-((2R,3R,4S,5R)-3,4-dihydroxy-5-(hydroxymethyl)tetrahydrofuran-2-yl)-6,7-dihydroxy-6-methyl-6,7-dihydro-3H-imidazo[1,2-a]purin-9(5H)-one (21), with the proviso that the TCR composed of SEQ ID NO 16 and 28 is disclaimed;
u. 3-((2R,3R,4S,5R)-3,4-dihydroxy-5-(hydroxymethyl)tetrahydrofuran-2-yl)-6,7-dihydroxy-7-methyl-6,7-dihydro-3H-imidazo[1,2-a]purin-9(5H)-one (22), with the proviso that the TCR composed of SEQ ID NO 16 and 28 is disclaimed;
v. 1-methylguanosine (24), with the proviso that the TCR composed of SEQ ID NO 22 and 34 is disclaimed;
w. 3-((2R,5R)-4-hydroxy-5-(hydroxymethyl) tetrahydrofuran-2-yl)-7-(2-oxoheptyl)-3H-imidazo[1,2-a]purin-9(5H)-one (25);
x. 2′-O-methylcytidine (29);
v. 3-methyluridine (30), with the proviso that the TCR composed of SEQ ID NO 22 and 34 is disclaimed;
z. 5-methyluridine (31);
aa. 3,2′-O-dimethyluridine (32);
bb. queuosine (33);
cc. wybutosine (34);
dd. hydroxywybutosine (35);
ee. pseudouridine (36);
ff. 6-((2R,4S,5R)-4-hydroxy-5-(hydroxymethyl)tetrahydrofuran-2-yl)-3-(2-oxoheptyl)-1,8a-dihydroimidazo [1,2-c]pyrimidin-5(6H)-one (37);
qq. N4-(3-oxo-1-propenyl)-2′-deoxycytidine (38), with the proviso that the TCR composed of SEQ ID NO 22 and 34 is disclaimed;
hh. 6-methylmercaptopurine (40), with the proviso that the TCRs composed of SEQ ID NO 16 and 28, 22 and 34, and of 24 and 36 are disclaimed;
ii. N6-methyladenosine (42);
jj. 6-Methylpurine (43);
kk. 6-(Dimethylamino)purine (44);
ll. N6-(A2-Isopentenyl) adenine (45);
mm. 1-Methylguanine (47);
nn. N2-Methyl-2′-deoxyguanosine (48);
oo. 5′-Deoxy-5′-(methylthio)adenosine (52);
pp. N⁶-Methyl-2′-deoxyadenosine (53), with the proviso that the TCR composed of SEQ ID NO 3 and 4 is disclaimed; and
qq. N⁶-(2-Hydroxyethyl)-2′-deoxyadenosine (54), with the proviso that the TCR composed of SEQ ID NO 3 and 4 is disclaimed.

17. (canceled)

18. (canceled)

19. The isolated T cell receptor (TCR) according to claim 16, wherein the TCR recognizes the following compound in association with MR1:
a. N6-isopentenyladenosine (6), particularly wherein the TCR comprises the CDRs comprised of SEQ ID 99 and 100, particularly wherein the TCR comprises the polypeptide chains characterized by SEQ ID 97 and 98;
b. NP-(3-Oxo-1-propenyl)-2′-deoxyadenosine (12), particularly wherein the TCR comprises the CDRs comprised of SEQ ID 99 and 100, or 103 and 104, or 108 and 108, or 111 and 112, or 123 and 124, particularly wherein the TCR comprises the polypeptide chains characterized by SEQ ID 97 and 98, or 101 and 102, or 105 and 106, or 109 and 110, or 121 and 122;
c. 8-(9H-purin-6-yl)-2-oxa-8-azabicyclo[3.3.1]nona-3,6-diene-4,6-dicarbaldehyde (13), particularly wherein the TCR comprises the CDRs comprised of SEQ ID 99 and 100, or 103and 104, or 107and 108, or 111 and 112, or 123and 124, particularly wherein the TCR comprises the polypeptide chains characterized by SEQ ID 97 and 98, or 101 and 102, or 105 and 106, or 109 and 110, or 121 and 122;
d. Pyrimido[1,2-a]purin-10(3H)-one (24), particularly wherein the TCR comprises the CDRs comprised of SEQ ID 99 and 100, or 103 and 104, or 111 and 112, particularly wherein the TCR comprises the polypeptide chains characterized by SEQ ID 97 and 98, or 101 and 102, or 109 and 110;
e. N⁴-(3-oxo-1-propenyl)-2′-deoxycytidine (38), particularly wherein the TCR comprises the CDRs comprised of SEQ ID 111 and 112, or 123 and 124, particularly wherein the TCR comprises the polypeptide chains characterized by SEQ ID 109 and 110, or 121 and 122;
f. 6-Methylmercaptopurine (40), particularly wherein the TCR comprises the CDRs comprised of SEQ ID 107 and 108, particularly wherein the TCR comprises the polypeptide chains characterized by SEQ ID 105 and 106.

20. An isolated T cell receptor (TCR) protein heterodimer comprising a TCR α chain and a TCR β chain, the TCR α chain and the TCR β chain each being characterized by a CDR3 sequence and the TCR protein heterodimer being characterized by a pair of α chain and β chain sequences selected from SEQ ID Nos 99 and 100, 103 and 104, 107 and 108, 111 and 112, 115 and 116, 119 and 120, 123 and 124, 127 and 128, 131 and 132;
particularly wherein the TCR α chain and the TCR β chain are selected from the pairs of α chain and β chain amino acid sequences of SEQ ID Nos 97 and 98, 101 and 102, 105 and 106, 109 and 110, 113 and 114, 117 and 118, 121 and 122,125 and 126, and of 129 and 130,
or a sequence at least 85% (≥90%, 95%, 98%) identical to said pair of α chain and β chain amino acid sequences, and having the same biological activity.

21. A polynucleotide encoding a TCR as claimed in claim 16, particularly wherein the polynucleotide is selected from
a. a DNA expression vector;
b. an RNA molecule, particularly a stabilized messenger RNA molecule
c. a viral vector.

22. An isolated T cell expressing, particularly expressing recombinantly, the TCR according to claim 16.

23. The isolated T cell according to claim 22, for use in prophylaxis or treatment of a disease associated with an aberrant or absent MR1-specific T cell response, particularly for use in treatment of cancer.

24. The isolated T cell and/or the polynucleotide for use according to claim 23, wherein the disease is cancer characterized by MR1 expression, and wherein the isolated T cell and/or the polynucleotide are co-administered with the MR1 ligand compound.

25. (canceled)

26. An MR1 ligand compound as specified in claim 1 for use in the treatment of cancer, wherein the MR1 ligand compound is administered in association with (administered prior to, concomitant with or after administration of) an isolated T cell expressing an MR1 specific TCR comprising a pair of □ and □ CDR3 sequences identified by the same line of Table 3A, particularly an MR1 specific TCR constituted by SEQ ID NO 1 and 2, 3 and 4, 5 and 6, 13 and 25, 14 and 26, 15 and 27, 16 and 28, 17 and 29, 18 and 30, 19 and 31, 20 and 32, 21 and 33, 22 and 34, 23 and 35, 24 and 36 and 61 and 62 and/or a polynucleotide encoding said MR1 specific TCR.

27. The isolated T cell and/or the polynucleotide for use according to claim 23, wherein the isolated T cell expressing the TCR and/or the polynucleotide is co-administered with a pharmaceutical compound selected from paclitaxel, doxorubicin, docetaxel, cabazitaxel, daunorubicin, epirubicin, idarubicin, disulfiram, ellagic acid, pentostatin and mycophenolic acid (MPA) amodiaquine, chlorpromazine, domperidone, estradiol, felopidine, loratadine, maprotiline, metoclopramide, nortriptyline, ondansetron, perphenazine, promazine, promethazine, raloxifene, salmeterol, tacrine, tamoxifen, and thioridazine, allopurinol, febuxostat, tisopurine, topiroxostat, inositols (phytic acid and myo-inositol),
particularly wherein the isolated T cell expressing the TCR and/or the polynucleotide is co-administered with a pharmaceutical compound selected from paclitaxel, doxorubicin, disulfiram, and MPA,
more particularly wherein the co-administered pharmaceutical compound is selected from paclitaxel and doxorubicin,
for treatment or prevention of a disease associated with aberrant or lacking MR1 expression, particularly treatment or prevention of recurrence of cancer disease associated with tumor cells expressing MR1.

28. A compound consisting of 8-(9H-purin-6-yl)-2-oxa-8-azabicyclo[3.3.1]nona-3,6-diene-4,6-dicarbaldehyde.


SEQUENCE LISTING (167 sequences)

SEQ ID NO: 1 - protein, 280 aa, Homo sapiens
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAAQIYNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 2 - protein, 309 aa, Homo sapiens
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSFSSGKQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 3 - protein, 269 aa, Homo sapiens
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVTGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 4 - protein, 313 aa, Homo sapiens
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSDVGTGDTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 5 - protein, 277 aa, Homo sapiens
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEEPSNTGKLIFGQGTTLQVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 6 - protein, 313 aa, Homo sapiens
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSRLLAGGQNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 7 - DNA, 840 bp, Homo sapiens
atggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcccagatttataaccagggaggaaagcttatcttcggacagggaacggagttatctgtgaaacccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 8 - DNA, 927 bp, Homo sapiens
atgggaatcaggctcctgtgtcgtgtggccttttgtttcctggctgtaggcctcgtagatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcagtttttctagcggaaagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 9 - DNA, 807 bp, Homo sapiens
atgatatccttgagagttttactggtgatcctgtggcttcagttaagctgggtttggagccaacggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtcggtatactccagtggtaatgaagatggaaggtttacagcacagctcaatagagccagccagtatatttccctgctcatcagagactccaagctcagtgattcagccacctacctctgtgtggtgaccggtaaccagttctattttgggacagggacaagtttgacggtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 10 - DNA, 939 bp, Homo sapiens
atggcctccctgctcttcttctgtggggccttttatctcctgggaacagggtccatggatgctgatgttacccagaccccaaggaataggatcacaaagacaggaaagaggattatgctggaatgttctcagactaagggtcatgatagaatgtactggtatcgacaagacccaggactgggcctacggttgatctattactcctttgatgtcaaagatataaacaaaggagagatctctgatggatacagtgtctctcgacaggcacaggctaaattctccctgtccctagagtctgccatccccaaccagacagctctttacttctgtgccaccagtgatgtcgggacaggggacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 11 - DNA, 831 bp, Homo sapiens
atgctgactgccagcctgttgagggcagtcatagcctccatctgtgttgtatccagcatggctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtgaggaacctagcaacacaggcaaactaatctttgggcaagggacaactttacaagtaaaaccagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 12 - DNA, 939 bp, Homo sapiens
atgggaatcaggctcctgtgtcgtgtggccttttgtttcctggctgtaggcctcgtagatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcagtcgactactagcgggggggcagaatgagcagttcttcgggccagggacacggctcaccgtgctagaggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 13 - protein, 267 aa, Homo sapiens
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVMDSSYKLIFGSGTRLLVRPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 14 - protein, 271 aa, Homo sapiens
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAAAGGTSYGKLTFGQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 15 - protein, 277 aa, Homo sapiens
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAETWTDRGSTLGRLYFGRGTQLTVWPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 16 - protein, 280 aa, Homo sapiens
MAMLLGASVLILWLQTDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASLYNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 17 - protein, 274 aa, Homo sapiens
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCASGDSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 18 - protein, 276 aa, Homo sapiens
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALTIWDYGGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 19 - protein, 269 aa, Homo sapiens
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAGENSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 20 - protein, 275 aa, Homo sapiens
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSLSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 21 - protein, 275 aa, Homo sapiens
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGQLGGAGGTSYGKLTFGQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 22 - protein, 274 aa, Homo sapiens
MTSIRAVFIFLWLQLDLVNGENVEQHPSTLSVQEGDSAVIKCTYSDSASNYFPWYKQELGKGPQLIIDIRSNVGEKKDQRIAVTLNKTAKHFSLHITETQPEDSAVYFCAANWSPQGNEKLTFGTGTRLTIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 23 - protein, 267 aa, Homo sapiens
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCASMDSNYQLIWGAGTKLIIKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 24 - protein, 274 aa, Homo sapiens
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNRFTRDGNKLVFGAGTILRVKSYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 25 - protein, 312 aa, Homo sapiens
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSEVTGGYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 26 - protein, 311 aa, Homo sapiens
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVGAGQGPYTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 27 - protein, 311 aa, Homo sapiens
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLGATGANEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 28 - protein, 308 aa, Homo sapiens
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSYRGTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 29 - protein, 309 aa, Homo sapiens
MGPGLLCWVLLCLLGAGPVDAGVTQSPTHLIKTRGQHVTLRCSPISGHKSVSWYQQVLGQGPQFIFQYYEKEERGRGNFPDRFSARQFPNYSSELNVNALLLGDSALYLCASSFDVGLPPLHFGNGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 30 - protein, 309 aa, Homo sapiens
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSREWETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 31 - protein, 315 aa, Homo sapiens
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSQLYRDTSNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 32 - protein, 313 aa, Homo sapiens
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASGISGTASSYNSPLHFGNGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 33 - protein, 312 aa, Homo sapiens
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVGGGLADTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 34 - protein, 311 aa, Homo sapiens
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSEYIQYSGNTIYFGEGSWLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 35 - protein, 314 aa, Homo sapiens
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSAKVTSGQHQGTTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 36 - protein, 307 aa, Homo sapiens
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVEGRGYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 37 - DNA, 801 bp, Homo sapiens
atgtggggagttttccttctttatgtttccatgaagatgggaggcactacaggacaaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgtgatggatagcagctataaattgatcttcgggagtgggaccagactgctggtcaggcctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 38 - DNA, 813 bp, Homo sapiens
atgctactcatcacatcaatgttggtcttatggatgcaattgtcacaggtgaatggacaacaggtaatgcaaattcctcagtaccagcatgtacaagaaggagaggacttcaccacgtactgcaattcctcaactactttaagcaatatacagtggtataagcaaaggcctggtggacatcccgtttttttgatacagttagtgaagagtggagaagtgaagaagcagaaaagactgacatttcagtttggagaagcaaaaaagaacagctccctgcacatcacagccacccagactacagatgtaggaacctacttctgtgcggctgctggtggtactagctatggaaagctgacatttggacaagggaccatcttgactgtccatccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 39 - DNA, 831 bp, Homo sapiens
atgaagacatttgctggattttcgttcctgtttttgtggctgcagctggactgtatgagtagaggagaggatgtggagcagagtcttttcctgagtgtccgagagggagacagctccgttataaactgcacttacacagacagctcctccacctacttatactggtataagcaagaacctggagcaggtctccagttgctgacgtatattttttcaaatatggacatgaaacaagaccaaagactcactgttctattgaataaaaaggataaacatctgtctctgcgcattgcagacacccagactggggactcagctatctacttctgtgcagagacctggaccgacagaggctcaaccctggggaggctatactttggaagaggaactcagttgactgtctggcctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgttgcggctgtggtccagc

SEQ ID NO: 40 - DNA, 840 bp, Homo sapiens
atggccatgctcctgggggcatcagtgctgattctgtggcttcagacagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaagtctttataaccagggaggaaagcttatcttcggacagggaacggagttatctgtgaaacccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 41 - DNA, 822 bp, Homo sapiens
atggagaagaatcctttggcagccccattactaatcctctggtttcatcttgactgcgtgagcagcatactgaacgtggaacaaagtcctcagtcactgcatgttcaggagggagacagcaccaatttcacctgcagcttcccttccagcaatttttatgccttacactggtacagatgggaaactgcaaaaagccccgaggccttgtttgtaatgactttaaatggggatgaaaagaagaaaggacgaataagtgccactcttaataccaaggagggttacagctatttgtacatcaaaggatcccagcctgaagactcagccacatacctctgtgcctcgggggattccgggtatgcactcaacttcggcaaaggcacctcgctgttggtcacaccccatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 42 - DNA, 828 bp, Homo sapiens
atgaactattctccaggcttagtatctctgatactcttactgcttggaagaacccgtggaaattcagtgacccagatggaagggccagtgactctctcagaagaggccttcctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaagaaaccacttctttccacttggagaaaggctcagttcaagtgtcagactcagcggtgtacttctgtgctctgacaatatgggattatggaggaagccaaggaaatctcatctttggaaaaggcactaaactctctgttaaaccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 43 - DNA, 807 bp, Homo sapiens
atggtcctgaaattctccgtgtccattctttggattcagttggcatgggtgagcacccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcagcccagcctggtgatacaggcctctacctctgtgcaggagaaaattccgggtatgcactcaacttcggcaaaggcacctcgctgttggtcacaccccatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 44 - DNA, 825 bp, Homo sapiens
atgatgaaatccttgagagttttactggtgatcctgtggcttcagttaagctgggtttggagccaacagaaggaggtggagcaggatcctggaccactcagtgttccagagggagccattgtttctctcaactgcacttacagcaacagtgcttttcaatacttcatgtggtacagacagtattccagaaaaggccctgagttgctgatgtacacatactccagtggtaacaaagaagatggaaggtttacagcacaggtcgataaatccagcaagtatatctccttgttcatcagagactcacagcccagtgattcagccacctacctctgtgcaatgagcctatcaggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 45 - DNA, 825 bp, Homo sapiens
atgctccttgaacatttattaataatcttgtggatgcagctgacatgggtcagtggtcaacagctgaatcagagtcctcaatctatgtttatccaggaaggagaagatgtctccatgaactgcacttcttcaagcatatttaacacctggctatggtacaagcaggaccctggggaaggtcctgtcctcttgatagccttatataaggctggtgaattgacctcaaatggaagactgactgctcagtttggtataaccagaaaggacagcttcctgaatatctcagcatccatacctagtgatgtaggcatctacttctgtgctgggcagctaggaggggctggtggtactagctatggaaagctgacatttggacaagggaccatcttgactgtccatccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 46 - DNA, 822 bp, Homo sapiens
atgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaggacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaaactggagcccgcaaggaaatgagaaattaacctttgggactggaacaagactcaccatcatacccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 47 - DNA, 801 bp, Homo sapiens
atgtggggagttttccttctttatgtttccatgaagatgggaggcactacaggacaaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgcttccatggatagcaactatcagttaatctggggcgctgggaccaagctaattataaagccagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 48 - DNA, 822 bp, Homo sapiens
atgatatccttgagagttttactggtgatcctgtggcttcagttaagctgggtttggagccaacggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaatgaagatggaaggtttacagcacagctcaatagagccagccagtatatttccctgctcatcagagactccaagctcagtgattcagccacctacctctgtgtggtgaacagattcacaagggatggaaacaaactggtctttggcgcaggaaccattctgagagtcaagtcctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 49 - DNA, 936 bp, Homo sapiens
atgagcatcggcctcctgtgctgtgtggccttttctctcctgtgggcaagtccagtgaatgctggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttctgtgccagcagtgaggtgacagggggatacaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 50 - DNA, 933 bp, Homo sapiens
atgctgagtcttctgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttggggcggggcaaggaccttacacagatacgcagtattttggcccaggcacccggctgacagtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 51 - DNA, 933 bp, Homo sapiens
atgggaatcaggctcctgtgtcgtgtggccttttgtttcctggctgtaggcctcgtagatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcagcttaggggcgacaggggctaatgaaaaactgttttttggcagtggaacccagctctctgtcttggaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 52 - DNA, 924 bp, Homo sapiens
atgggctcctggaccctctgctgtgtgtccctttgcatcctggtagcaaagcacacagatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagctacaggggcactgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 53 - DNA, 927 bp, Homo sapiens
atgggccctgggctcctctgctgggtgctgctttgtctcctgggagcaggcccagtggacgctggagtcacccaaagtcccacacacctgatcaaaacgagaggacagcacgtgactctgagatgctctcctatctctgggcacaagagtgtgtcctggtaccaacaggtcctgggtcaggggccccagtttatctttcagtattatgagaaagaagagagaggaagaggaaacttccctgatcgattctcagctcgccagttccctaactatagctctgagctgaatgtgaacgccttgttgctgggggactcggccctgtatctctgtgccagcagctttgacgttggtttgccacccctccactttgggaacgggaccaggctcactgtgacagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 54 - DNA, 927 bp, Homo sapiens
atgggtcctgggcttctccactggatggccctttgtctccttggaacaggtcatggggatgccatggtcatccagaacccaagataccaggttacccagtttggaaagccagtgaccctgagttgttctcagactttgaaccataacgtcatgtactggtaccagcagaagtcaagtcaggccccaaagctgctgttccactactatgacaaagattttaacaatgaagcagacacccctgataacttccaatccaggaggccgaacacttctttctgctttcttgacatccgctcaccaggcctgggggacgcagccatgtacctgtgtgccaccagcagagagtgggagacccagtacttcgggccaggcacgcggctcctggtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 55 - DNA, 945 bp, Homo sapiens
atgactatcaggctcctctgctacatgggcttttattttctgggggcaggcctcatggaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctctgtgccagcagccaactttaccgggacacctcgaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 56 - DNA, 939 bp, Homo sapiens
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcggaatcagcgggacagcgagctcctataattcacccctccactttgggaacgggaccaggctcactgtgacagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 57 - DNA, 936 bp, Homo sapiens
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgtcggagggggattggcagatacgcagtattttggcccaggcacccggctgacagtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 58 - DNA, 933 bp, Homo sapiens
atgactatcaggctcctctgctacatgggcttttattttctgggggcaggcctcatggaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctctgtgccagcagtgaatatatccagtactctggaaacaccatatattttggagagggaagttggctcactgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 59 - DNA, 942 bp, Homo sapiens
atgctgctgcttctgctgcttctggggccaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgcgaaggtgactagcgggcaacaccaagggaccacagatacgcagtattttggcccaggcacccggctgacagtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 60 - DNA, 921 bp, Homo sapiens
atgctgagtcttctgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgaaggcaggggttacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 61 - protein, 310 aa, Homo sapiens
MQWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWETQELGKKIKVFGPGTKLIITDKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 62 - protein, 295 aa, Homo sapiens
MLFSSLLCVFVAFSYSGSSVAQKVTQAQSSVSMPVRKAVTLNCLYETSWWSYYIFWYKQLPSKEMIFLIRQGSDEQNAKSGRYSVNFKKAVKSVALTISALQLEDSAKYFCALGVQALLPILGDTTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 63 - DNA, 930 bp, Homo sapiens
atgcagtgggccctagcggtgcttctagctttcctgtctcctgccagtcagaaatcttccaacttggaagggagaacgaagtcagtcatcaggcagactgggtcatctgctgaaatcacttgtgatcttgctgaaggaagtaccggctacatccactggtacctacaccaggaggggaaggccccacagcgtcttctgtactatgactcctacacctccagcgttgtgttggaatcaggaatcagcccagggaagtatgatacttatggaagcacaaggaagaacttgagaatgatactgcgaaatcttattgaaaatgactctggagtctattactgtgccacctgggaaactcaagagttgggcaaaaaaatcaaggtatttggtcccggaacaaagcttatcattacagataaacaacttgatgcagatgtttcccccaagcccactatttttcttccttcaattgctgaaacaaagctccagaaggctggaacatacctttgtcttcttgagaaatttttccctgatgttattaagatacattggcaagaaaagaagagcaacacgattctgggatcccaggaggggaacaccatgaagactaacgacacatacatgaaatttagctggttaacggtgccagaaaagtcactggacaaagaacacagatgtatcgtcagacatgagaataataaaaacggagttgatcaagaaattatctttcctccaataaagacagatgtcatcacaatggatcccaaagacaattgttcaaaagatgcaaatgatacactactgctgcagctcacaaacacctctgcatattacatgtacctcctcctgctcctcaagagtgtggtctattttgccatcatcacctgctgtctgcttagaagaacggctttctgctgcaatggagagaaatca

SEQ ID NO: 64 - DNA, 885 bp, Homo sapiens
atgctgttctccagcctgctgtgtgtatttgtggccttcagctactctggatcaagtgtggcccagaaggttactcaagcccagtcatcagtatccatgccagtgaggaaagcagtcaccctgaactgcctgtatgaaacaagttggtggtcatattatattttttggtacaagcaacttcccagcaaagagatgattttccttattcgccagggttctgatgaacagaatgcaaaaagtggtcgctattctgtcaacttcaagaaagcagtgaaatccgtcgccttaaccatttcagccttacagctagaagattcagcaaagtacttttgtgctcttggggtccaagccctcctacccatactgggggataccaccgataaactcatctttggaaaaggaacccgtgtgactgtggaaccaagaagtcagcctcataccaaaccatccgtttttgtcatgaaaaatggaacaaatgtcgcttgtctggtgaaggaattctaccccaaggatataagaataaatctcgtgtcatccaagaagataacagagtttgatcctgctattgtcatctctcccagtgggaagtacaatgctgtcaagcttggtaaatatgaagattcaaattcagtgacatgttcagttcaacacgacaataaaactgtgcactccactgactttgaagtgaagacagattctacagatcacgtaaaaccaaaggaaactgaaaacacaaagcaaccttcaaagagctgccataaacccaaagccatagttcataccgagaaggtgaacatgatgtccctcacagtgcttgggctacgaatgctgtttgcaaagactgttgccgtcaattttctcttgactgccaagttatttttcttg

SEQ ID NO: 65 - protein, 17 aa, Homo sapiens
CAAQIYNQGGKLIFGQG

SEQ ID NO: 66 - protein, 13 aa, Homo sapiens
CVVTGNQFYFGTG

SEQ ID NO: 67 - protein, 18 aa, Homo sapiens
CALSEEPSNTGKLIFGQG

SEQ ID NO: 68 - protein, 15 aa, Homo sapiens
CAVMDSSYKLIFGSG

SEQ ID NO: 69 - protein, 17 aa, Homo sapiens
CAAAGGTSYGKLTFGQG

SEQ ID NO: 70 - protein, 20 aa, Homo sapiens
CAETWTDRGSTLGRLYFGRG

SEQ ID NO: 71 - protein, 17 aa, Homo sapiens
CAASLYNQGGKLIFGQG

SEQ ID NO: 72 - protein, 15 aa, Homo sapiens
CASGDSGYALNFGKG

SEQ ID NO: 73 - protein, 20 aa, Homo sapiens
CALTIWDYGGSQGNLIFGKG

SEQ ID NO: 74 - protein, 15 aa, Homo sapiens
CAGENSGYALNFGKG

SEQ ID NO: 75 - protein, 17 aa, Homo sapiens
CAMSLSGGSYIPTFGRG

SEQ ID NO: 76 - protein, 21 aa, Homo sapiens
CAGQLGGAGGTSYGKLTFGQG

SEQ ID NO: 77 - protein, 18 aa, Homo sapiens
CAANWSPQGNEKLTFGTG

SEQ ID NO: 78 - protein, 15 aa, Homo sapiens
CASMDSNYQLIWGAG

SEQ ID NO: 79 - protein, 18 aa, Homo sapiens
CVVNRFTRDGNKLVFGAG

SEQ ID NO: 80 - protein, 15 aa, Homo sapiens
CASSFSSGKQYFGPG

SEQ ID NO: 81 - protein, 19 aa, Homo sapiens
CATSDVGTGDTGELFFGEG

SEQ ID NO: 82 - protein, 19 aa, Homo sapiens
CASSRLLAGGQNEQFFGPG

SEQ ID NO: 83 - protein, 18 aa, Homo sapiens
CASSEVTGGYNEQFFGPG

SEQ ID NO: 84 - protein, 19 aa, Homo sapiens
CSVGAGQGPYTDTQYFGPG

SEQ ID NO: 85 - protein, 19 aa, Homo sapiens
CASSLGATGANEKLFFGSG

SEQ ID NO: 86 - protein, 15 aa, Homo sapiens
CASSYRGTEAFFGQG

SEQ ID NO: 87 - protein, 17 aa, Homo sapiens
CASSFDVGLPPLHFGNG

SEQ ID NO: 88 - protein, 15 aa, Homo sapiens
CATSREWETQYFGPG

SEQ ID NO: 89 - protein, 21 aa, Homo sapiens
CASSQLYRDTSNTGELFFGEG

SEQ ID NO: 90 - protein, 21 aa, Homo sapiens
CASGISGTASSYNSPLHFGNG

SEQ ID NO: 91 - protein, 18 aa, Homo sapiens
CASSVGGGLADTQYFGPG

SEQ ID NO: 92 - protein, 19 aa, Homo sapiens
CASSEYIQYSGNTIYFGEG

SEQ ID NO: 93 - protein, 22 aa, Homo sapiens
CSAKVTSGQHQGTTDTQYFGPG

SEQ ID NO: 94 - protein, 15 aa, Homo sapiens
CSVEGRGYEQYFGPG

SEQ ID NO: 95 - protein, 19 aa, Homo sapiens
CATWETQELGKKIKVFGPG

SEQ ID NO: 96 - protein, 24 aa, Homo sapiens
CALGVQALLPILGDTTDKLIFGKG

SEQ ID NO: 97 - protein, 276 aa, Homo sapiens
MLLLLVPVLEVIFTLGGTRAQSVTQLDSHVSVSEGTPVLLRCNYSSSYSPSLFWYVQHPNKGLQLLLKYTSAATLVKGINGFEAEFKKSETSFHLTKPSAHMSDAAEYFCVVSDDPLSGNEKLTFGTGTRLTIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 98 - protein, 315 aa, Homo sapiens
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSFSGGISSSYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 99 - protein, 13 aa, Homo sapiens
SDDPLSGNEKLTF

SEQ ID NO: 100 - protein, 15 aa, Homo sapiens
SFSGGISSSYNEQFF

SEQ ID NO: 101 - protein, 280 aa, Homo sapiens
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASYYNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 102 - protein, 310 aa, Homo sapiens
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSPGYQETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 103 - protein, 11 aa, Homo sapiens
SYYNQGGKLIF

SEQ ID NO: 104 - protein, 10 aa, Homo sapiens
SPGYQETQYF

SEQ ID NO: 105 - protein, 277 aa, Homo sapiens
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEWNQGAQKLVFGQGTRLTINPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 106 - protein, 315 aa, Homo sapiens
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSLMSVGGSAGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 107 - protein, 10 aa, Homo sapiens
WNQGAQKLVF

SEQ ID NO: 108 - protein, 14 aa, Homo sapiens
SLMSVGGSAGELFF

SEQ ID NO: 109 - protein, 273 aa, Homo sapiens
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMREALGFVFGPGTRLSVLPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 110 - protein, 312 aa, Homo sapiens
MSLGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSPLEGSYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 111 - protein, 8 aa, Homo sapiens
REALGFVF

SEQ ID NO: 112 - protein, 12 aa, Homo sapiens
SPLEGSYNEQFF

SEQ ID NO: 113 - protein, 277 aa, Homo sapiens
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVSDADRGSTLGRLYFGRGTQLTVWPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 114 - protein, 309 aa, Homo sapiens
MASLLFFCGAFYLLGTGSMDADVTQTPRNRITKTGKRIMLECSQTKGHDRMYWYRQDPGLGLQLIYYSFDVKDINKGEISDGYSVSRQAQAKFSLSLESAIPNQTALYFCATSDRGNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 115 - protein, 14 aa, Homo sapiens
SDADRGSTLGRLYF

SEQ ID NO: 116 - protein, 9 aa, Homo sapiens
SDRGNEQFF

SEQ ID NO: 117 - protein, 273 aa, Homo sapiens
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVGQAGTALIFGKGTTLSVSSNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 118 - protein, 314 aa, Homo sapiens
MGCRLLCCAVLCLLGAVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDRNDRGYVNYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 119 - protein, 9 aa, Homo sapiens
GQAGTALIF

SEQ ID NO: 120 - protein, 16 aa, Homo sapiens
SQDRNDRGYVNYGYTF

SEQ ID NO: 121 - protein, 277 aa, Homo sapiens
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALNLFITWGGSNYKLTFGKGTLLTVNPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 122 - protein, 313 aa, Homo sapiens
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASCADLATVGETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 123 - protein, 15 aa, Homo sapiens
NLFITWGGSNYKLTF

SEQ ID NO: 124 - protein, 13 aa, Homo sapiens
CADLATVGETQYF

SEQ ID NO: 125 - protein, 268 aa, Homo sapiens
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAGTNDYKLSFGAGTTVTVRANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 126 - protein, 313 aa, Homo sapiens
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARGDRGLAGIYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 127 - protein, 8 aa, Homo sapiens
TNDYKLSF

SEQ ID NO: 128 - protein, 15 aa, Homo sapiens
RGDRGLAGIYNEQFF

SEQ ID NO: 129 - protein, 273 aa, Homo sapiens
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAGHDSLSNSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 130 - protein, 314 aa, Homo sapiens
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVSLGLLAGEGAFYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 131 - protein, 13 aa, Homo sapiens
HDSLSNSGYALNF

SEQ ID NO: 132 - protein, 16 aa, Homo sapiens
SLGLLAGEGAFYEQYF

SEQ ID NO: 133 - DNA, 831 bp, Homo sapiens
atgctcctgctgctcgtcccagtgctcgaggtgatttttaccctgggaggaaccagagcccagtcggtgacccagcttgacagccacgtctctgtctctgaaggaaccccggtgctgctgaggtgcaactactcatcttcttattcaccgtctctcttctggtatgtgcaacaccccaacaaaggactccagcttctcctgaagtacacatcagcggccaccctggttaaaggtatcaacggttttgaggctgaatttaagaagagtgaaacctccttccacctgacgaaaccctcagcccatatgagcgacgcggctgagtacttctgtgttgtgagtgatgaccccttgtccggaaatgagaaattaacctttgggactggaacaagactcaccatcatacccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 134 - DNA, 948 bp, Homo sapiens
atgggccctgggctcctctgctgggcactgctttgtctcctgggagcaggcttagtggacgctggagtcacccaaagtcccacacacctgatcaaaacgagaggacagcaagtgactctgagatgctctcctaagtctgggcatgacactgtgtcctggtaccaacaggccctgggtcaggggccccagtttatctttcagtattatgaggaggaagagagacagagaggcaacttccctgatcgattctcaggtcaccagttccctaactatagctctgagctgaatgtgaacgccttgttgctgggggactcggccctctatctctgtgccagcagcttttcaggaggaatatctagctcctacaatgagcagttcttcgggccagggacacggctcaccgtgctagaggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 135 - DNA, 843 bp, Homo sapiens
atggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgttttcttaaacaaaagtgccaagcacctctctctgcacattgtgccctcccagcctggagactctgcagtgtacttctgtgcagcaagttattataaccagggaggaaagcttatcttcggacagggaacggagttatctgtgaaacccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggttgtggtccagctga

SEQ ID NO: 136 - DNA, 933 bp, Homo sapiens
atgggaatcaggctcctgtgtcgtgtggccttttgtttcctggctgtaggcctcgtagatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcagccccggatatcaagagacccagtacttcgggccaggcacgcggctcctggtgctcgaggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 137 - DNA, 834 bp, Homo sapiens
atgctgactgccagcctgttgagggcagtcatagcctccatctgtgttgtatccagcatggctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtgagtggaatcagggagcccagaagctggtatttggccaaggaaccaggctgactatcaacccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 138 - DNA, 948 bp, Homo sapiens
atgggctcctggaccctctgctgtgtgtccctttgcatcctggtagcaaagcacacagatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtttaatgagcgtcgggggcagcgccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 139 - DNA, 822 bp, Homo sapiens
atgtcactttctagcctgctgaaggtggtcacagcttcactgtggctaggacctggcattgcccagaagataactcaaacccaaccaggaatgttcgtgcaggaaaaggaggctgtgactctggactgcacatatgacaccagtgatcaaagttatggtctattctggtacaagcagcccagcagtggggaaatgatttttcttatttatcaggggtcttatgacgagcaaaatgcaacagaaggtcgctactcattgaatttccagaaggcaagaaaatccgccaaccttgtcatctccgcttcacaactgggggactcagcaatgtatttctgtgcaatgagagaggccctgggttttgtctttggtcccggaaccagattgtccgtgctgccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 140 - DNA, 939 bp, Homo sapiens
atgagcctcgggctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagccctttggaagggtcctacaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 141 - DNA, 834 bp, Homo sapiens
atggcctctgcacccatctcgatgcttgcgatgctcttcacattgagtgggctgagagctcagtcagtggctcagccggaagatcaggtcaacgttgctgaagggaatcctctgactgtgaaatgcacctattcagtctctggaaacccttatcttttttggtatgttcaataccccaaccgaggcctccagttccttctgaaatacatcacaggggataacctggttaaaggcagctatggctttgaagctgaatttaacaagagccaaacctccttccacctgaagaaaccatctgcccttgtgagcgactccgctttgtacttctgtgctgtgagcgacgccgacagaggctcaaccctggggaggctatactttggaagaggaactcagttgactgtctggcctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 142 - DNA, 930 bp, Homo sapiens
atggcctccctgctcttcttctgtggggccttttatctcctgggaacagggtccatggatgctgatgttacccagaccccaaggaataggatcacaaagacaggaaagaggattatgctggaatgttctcagactaagggtcatgatagaatgtactggtatcgacaagacccaggactgggcctacagttgatctattactcctttgatgtcaaagatataaacaaaggagagatctctgatggatacagtgtctctcgacaggcacaggctaaattctccctgtccctagagtctgccatccccaaccagacagctctttacttctgtgccaccagtgatcgaggaaatgagcagttcttcgggccagggacacggctcaccgtgctagaggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 143 - DNA, 822 bp, Homo sapiens
atgaaaaagcatctgacgaccttcttggtgattttgtggctttatttttatagggggaatggcaaaaaccaagtggagcagagtcctcagtccctgatcatcctggagggaaagaactgcactcttcaatgcaattatacagtgagccccttcagcaacttaaggtggtataagcaagatactgggagaggtcctgtttccctgacaatcatgactttcagtgagaacacaaagtcgaacggaagatatacagcaactctggatgcagacacaaagcaaagctctctgcacatcacagcctcccagctcagcgattcagcctcctacatctgtgtggtgggtcaggcaggaactgctctgatctttgggaagggaaccaccttatcagtgagttccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 144 - DNA, 945 bp, Homo sapiens
atgggctgcaggctgctctgctgtgcggttctctgtctcctgggagcggtccccatggaaacgggagttacgcagacaccaagacacctggtcatgggaatgacaaataagaagtctttgaaatgtgaacaacatctgggtcataacgctatgtattggtacaagcaaagtgctaagaagccactggagctcatgtttgtctacagtcttgaagaacgggttgaaaacaacagtgtgccaagtcgcttctcacctgaatgccccaacagctctcacttattccttcacctacacaccctgcagccagaagactcggccctgtatctctgcgccagcagccaagatcgaaacgacagggggtacgttaactatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 145 - DNA, 834 bp, Homo sapiens
atgaactattctccaggcttagtatctctgatactcttactgcttggaagaacccgtggaaattcagtgacccagatggaagggccagtgactctctcagaagaggccttcctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaagaaaccacttctttccacttggagaaaggctcagttcaagtgtcagactcagcggtgtacttctgtgctctgaatcttttcattacatggggaggtagcaactataaactgacatttggaaaaggaactctcttaaccgtgaatccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 146 - DNA, 942 bp, Homo sapiens
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagctgcgcggatctggccacagtaggggagacccagtacttcgggccaggcacgcggctcctggtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 147 - DNA, 807 bp, Homo sapiens
atgctactcatcacatcaatgttggtcttatggatgcaattgtcacaggtgaatggacaacaggtaatgcaaattcctcagtaccagcatgtacaagaaggagaggacttcaccacgtactgcaattcctcaactactttaagcaatatacagtggtataagcaaaggcctggtggacatcccgtttttttgatacagttagtgaagagtggagaagtgaagaagcagaaaagactgacatttcagtttggagaagcaaaaaagaacagctccctgcacatcacagccacccagactacagatgtaggaacctacttctgtgcagggaccaacgactacaagctcagctttggagccggaaccacagtaactgtaagagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 148 - DNA, 942 bp, Homo sapiens
atgctgctgcttctgctgcttctggggccaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctaggggggacaggggactagcggggatttacaatgagcagttcttcgggccagggacacggctcaccgtgctagaggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 149 - DNA, 822 bp, Homo sapiens
atggtcctgaaattctccgtgtccattctttggattcagttggcatgggtgagcacccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcggcccagcctggtgatacaggcctctacctctgtgcaggacatgatagcctttcaaattccgggtatgcactcaacttcggcaaaggcacctcgctgttggtcacaccccatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 150 - DNA, 945 bp, Homo sapiens
atgctgagtcttctgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgtttccctggggttgttagcgggagagggtgctttctacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 151 - DNA, 20 bp, Artificial Sequence
  gRNA
caggcttgatggatccgtct

SEQ ID NO: 152 - DNA, 20 bp, Artificial Sequence
  gRNA
tcaccgtactgtccacgccg

SEQ ID NO: 153 - DNA, 20 bp, Artificial Sequence
  gRNA
gcggtacagtccgcacctgc

SEQ ID NO: 154 - DNA, 20 bp, Artificial Sequence
  gRNA
ttccaggggggcaacaacgc

SEQ ID NO: 155 - DNA, 20 bp, Artificial Sequence
  gRNA
gctgatgatgtcggcgtccg

SEQ ID NO: 156 - DNA, 20 bp, Artificial Sequence
  gRNA
acataccgaagtcaatgtcg

SEQ ID NO: 157 - DNA, 20 bp, Artificial Sequence
  gRNA
acataccgaagtcaatgtcg

SEQ ID NO: 158 - DNA, 20 bp, Artificial Sequence
  gRNA
taccttgggatctctccgtt

SEQ ID NO: 159 - DNA, 20 bp, Artificial Sequence
  gRNA
tcaagaaaatctgcgtaggt

SEQ ID NO: 160 - DNA, 20 bp, Artificial Sequence
  gRNA
gaaccgcagcccccgtccgg

SEQ ID NO: 161 - DNA, 20 bp, Artificial Sequence
  gRNA
gtccggcggcctcacggacg

SEQ ID NO: 162 - DNA, 20 bp, Artificial Sequence
  gRNA
cggcgagggcttaccggtgt

SEQ ID NO: 163 - DNA, 20 bp, Artificial Sequence
  gRNA
accggtgtcggccggcacct

SEQ ID NO: 164 - DNA, 20 bp, Artificial Sequence
  gRNA
cgaagtcgatataggcagta

SEQ ID NO: 165 - DNA, 20 bp, Artificial Sequence
  gRNA
gtgtagttcgaccattcgtg

SEQ ID NO: 166 - DNA, 20 bp, Artificial Sequence
  gRNA
gttcaggatcacgttaccgc

SEQ ID NO: 167 - DNA, 20 bp, Artificial Sequence
  gRNA
aaatgtgagatcagagtaat